SEQUENCE LISTING (41 sequences)

SEQ ID NO: 1 - DNA, 1369 bp, Artificial Sequence
  Cochliobolus heterostrophus C4 cel7
cttcttggttctcaaagatgctctccaacgtctttcttaccgctgccctcgcagccggcctggctcaggccctgccccaggccacgcctaccccaaccgctgcgccctctggcaaccccttcgcgggcaagaacttctacgccaacccatactactcgtctgaagtccacaccctggccatgccctcgcttccagcctcgctgaagcctgctgctaccgccgtggccaaggtcggatcattcgtgtggatggacaccatggccaaggttcctctcatggacacctacctcgcagacatcaaggccaagaacgctgctggcgcaaacctcatgggtacttttgtcgtctacgaccttcccgaccgtgactgcgccgctctggcctccaacggtgaactcaagattgacgagggtggtgtcgagaagtacaagacccagtacattgacaagattgccgccatcatcaagaagtaccccgacgtcaagatcaaccttgccattgagcccgattcccttgccaacatggtcaccaacatgggtgtgcagaagtgctcgcgcgccgccccatactacaaggagctcactgcctacgccctcaagacgctcaacttcaacaacgtcgacatgtacatggacggtggccacgccggttggctcggctgggacgccaacattggccctaccgcaaagcttttcgcagaggtctacaaggctgctggctctccccgtggcgtccgtggtatcgtcaccaacgtcagcaactacaacgctctccgcgtctcctcctgcccatccatcacccaaggaaacaagaactgcgacgaggagcgctacatcaacgccttggctcctcttctcaagaacgagggtttccctgctcacttcatcgtcgaccagggccgctccggaaaggtgcctactaaccagcaggagtggggtgactggtgcaacgtctcaggtgctggattcggtacccgtcccaccaccaacactggcaatgccctcattgatgccatcgtctgggtcaagcccggtggcgagtctgacggtacctctgacaccagcgctgcccgctacgatgcccactgcggcaggaacagcgctttcaagcccgctcctgaggctggaacctggttccaggcttacttcgagatgcttctcaagaacgctaaccctgctcttgcttaagtgtctggttcttttgaataagcttgggtagattgttagaagggaaaattagtctgcgagtggtctttcaccgcagattctggtggattgtaaatatggctttggaactagaataggcaacgtttgatgttgcagttcgtgtaaatattataccttttggagctaaaaaaaaaaaaaaaaaa

SEQ ID NO: 2 - DNA, 1581 bp, Artificial Sequence
  Gibberella zeae K59 cel6
atgacggcctacaagcttttcctggctgctgcttttgcagccactgctctcgcagctcctgttgaagagcgtcagtcttgcagcaacggagtctggtgagtgtttgcagccatctttttaaagaattaattactcacatacccataggtctcaatgtggtggtcagaactggagcggtactccttgctgcaccagtggaaacaagtgtgtcaaggtcaacgacttctactcccaatgccagcctggatccgcagacccttctcccacgagcaccattgtcagtgccacaaccaccaaggctactaccactggtagtggaggctctgtcacctcgcctcctcctgttgccaccaacaatcccttctctggcgttgatctgtgggccaacaactactaccgctccgaggtcagcactctcgctatccccaagctgagcggtgccatggccaccgctgctgccaaggtcgccgatgttccttctttccagtggatgtgagttacgagtccctttggatatatacctctttactaaccacgatagggacacttatgaccacatctccttcatggaggactctcttgccgatatccgcaaggccaacaaggctggtggcaactacgctggtcagttcgtcgtctacgatcttcccgaccgtgactgtgctgctgctgcctccaacggagagtactcccttgacaaggatggcaagaacaagtacaaggcctacattgcagatcaagggatccttcaggactactctgacacccgcatcattctcgttatcggttagtccacctgattgactccgacttagttcctactaacagccatttagagcctgattctcttgctaacatggtcaccaacatgaacgtccccaagtgcgccaacgctgctagcgcttacaaggagctcaccattcacgccctcaaggagctcaaccttcccaacgtctccatgtacatcgatgcaggtcacggtggctggctgggatggcccgccaaccttcctcctgccgcccagctctacggtcagctctacaaggatgccggcaagccatctcgcctccgaggtctcgtcaccaacgtctccaactacaacgcctggaagctgtcctccaagcccgactacactgagagcaaccccaactacgacgagcagaagtacatccacgctctatctcctcttctggagcaggagggctggcccggtgccaagttcatcgtcgaccagggccgatctggtaagcagcccactggccagaaggcttggggtgactggtgcaacgctcccggaactggattcggtctccgaccctctgccaacactggcgatgccctcgtcgacgctttcgtctgggtcaagcctggtggtgagtctgatggtacctctgatacctctgctgctcgctacgactaccactgcggtattgacggcgctgtcaagtaagttttataatacaaatcctcaagttaaccctcatactaaccccgataactaggcccgctcctgaggctggaacctggttccaggcttactttgagcagcttctcaagaacgccaacccctctttcctgtaa

SEQ ID NO: 3 - DNA, 2493 bp, Artificial Sequence
  Irpex lacteus MC-2 cex3
ccgcaccccagcatagcaacagctttttcgtcggcaagatattaagcacggtcatggagttttcaacgacttaaccgagcttgtaccgaagtggacggcagttcgctgaacgttcgggtgtgctttttacaacccgtcgttgaaaataatgtgtaggtatggccgtagcctcatgaccccactcataacgtccgtcgttcagcaactgaccctcccccgacgtctatccgctaacaatgctcgggtctacgccggaattatggtattcttccactggtgggcctgaacgatgcaaaacggtgcttctgatgagcccacctctgtattatttccggtatataagaagtggtatcgtcggctagggttctacaggatccacatcccactgagacgaatccactgcaagtgcaatgaagtccgctgctttcctcgctgctctcgccgccatcctcccagcctatgtcgctggccaagcccagacttgggcacagtgcggtggtatcggcttcagtacgttactacctttctccttctactggtctgttacttactgaacttgcctatcatagctggtcctaccacttgcgttgccggctccgtctgcacgaagcagaatgattactactctcagtgcatgtaagtacgaatccacccttttgcaagaactactgacttatgatggggtatagtcctggatctgctactactcccacatctgcacctacatctgcacccacctcccagccttcgcagccatcttccacctcctctgctccttccggtccttcctctacccccacgccctctgccaacaacccatggactggctaccaggtatgcgggcgatccattgtaactctaaaaatctctttctgacctgacctgggcatagatctacttgagcccttactacgctaacgaggttgctgccgccgccaaggcaatcacggaccccaccctcgccgccaaggctgccagcgttgctaacatcccgaacttcacttggttgggtgagtgtgacattgacaagagaaggaaacgacttcctaattacccgcatagactccgtctccaagatcgctgatcttaagacatacctcgctgacgcaagtgcactgggcaagtccagcggtcagaagcaactcctccagattgtcgtatacgatcttcccgaccgtgattgcgctgctaaggcctccaatggagagttcagcattgctgacaacggcctggccaactaccagaactacatcgaccagatcgttgctgctgtcaagcgtaagtctcgacgaggcagttcacttcgctttgcatactgagcctgttcgccacagaattccctgacgttcgggtcgtggctgtcattgagcccgactctcttgccaacttggtcaccaacttgaacgtgcagaagtgcgctaacgccaagagcacctacctcactgccgtcaactacgctttgaagcagctctcctcagttggcgtgtaccagtacatggacgcaggtcacgccggatggctcggttggcccgccaacttgacccccgccgctcagctgttcgctcaagtttactctgatgccggaaagtcgccattcatcaagggtcttgctaccagtacgttttcatttcgttttgttcgatcactcaagactgacccgcttgaatcgcaaagacgtcgccaactacaacgccttgagcgcggcctcacccgatcccatcacccagggtgaccccaactacgatgaaatccactacatcaacgtaagcccgtttaaccgtacaatgcgatgtgtactaatcaaaccaaatcccgcaggctctcgctccggctctccagtccgctggcttccctgctaccttcatcgtcgatcaaggccgttccggtcagcagaaccaccgacaacagtggggtgactggtgcaacatcaagggtgctgggttcggtacccgcccgaccaccaacactggttcttcgctcatcgactccatcgtttgggtgaagcccggaggtgaatccgacggtacctcgaactcgtcttcgccccgtttcgactccacttgctctttggtaagttcggccttctgttcgtcaaactgagtgtgatgctaactcatcgtgcttgcagtcggatgctactcagcccgctcctgaggccggtacatggttccaggcttacttcgagactctcgtctccaaggccaacccaccgctctaagcgtatcgtacctgctttcaaaatgtggctgaacggcatagaacagctgctcttggggttctcttcacttgatcgcgatttttatatacctgtattttatgtagcataaaaagtaaaacagccgcagaaatgcattcgcttttcacttgtaccgcgtcttgttcttgtgccaaatgctctcgcgtcctaccgagttcatctttcgatatcagtgagcggccagcatcgaaacgaccactgcgttagtttgtctggcgacatctgcatgcaagcta

SEQ ID NO: 4 - DNA, 1453 bp, Artificial Sequence
  Volvariella volvacea cbhII-I
tgattgcaagccacatatcccagagatgtccaggttttctgctcttactgctctccttttatctttgccactactggctattgctcagtccccgttgtatgggcaatgtggtggcaacggctggactggcccaaagacctgtgtatcaggtgcaacttgtacagtgatcaatgactggtattggcaatgcctgccaggaaatggcccaacttcttcttcaccaacttccacacctaccaccaccacaactacagggggacctcaaccaaccgtaccagcagcagggaatccttatactggatacgagatttacttgagtccttattacgctgctgaggctcaagctgcggctgcccaaatttctgatgccacgcagaaggccaaagccctgaaggtcgcacaaatccccacattcacctggtttgatgttattgcaaagacctccacactcggtgattatttggccgaagcgagcgcacttgggaaatcctctggaaagaaatacctcgttcaaatcgttgtatatgacttgccagatcgggattgcgctgctctggcttcgaatggagagtttagcatcgcaaacaatgggctcaacaactacaagggctacatcgatcaattggttgctcagatcaagaaataccctgatgtccgagtcgtggctgttattgaacccgactccttggccaatctcgttaccaatctcaatgttagcaagtgtgccaatgcacaaacagcctacaaggctggtgtcacgtacgctctccagcagctcaactctgttggcgtctatatgtacctcgatgctggacatgcgggttggctcggatggcctgccaacttgaatcccgctgcgcaactgttctctcaattgtacagagatgctggaagtccccaatatgtccgtggcctagctaccaatgttgccaactacaacgcactctctgccagcagccccgacccagtcacacaaggcaatcccaactatgacgaacttcattacatcaacgcactcgcgccagctctccaatccggtggcttccctgcccacttcattgtcgaccaaggccgatcaggagttcagaacatcagacaacaatggggcgactggtgcaacgtcaagggtgcaggctttggccagcgtccaactcttagcacaggttcatcccttatcgacgccattgtctggattaaacccggaggcgaatgcgacggtacaaccaacacatcgtcacctcgctatgattctcactgtggtctttctgatgctacacccaatgccccagaagctggccaatggttccaggcttacttcgagaccttagtccgtaacgccagcccacctctttgagtgtgcagtgtagataccagatatacaaggccccgagtgtgatacaacagaataaataatccctttttgctcctctcaaaaaaaaaaaaaaaaaaaaaa

SEQ ID NO: 5 - DNA, 1920 bp, Artificial Sequence
  Piromyces sp. E2 cel6A
aaatcttaattataattaataatatcatttttcatttattatatttatactttgtttcatgaaataataataaacaacattttcccaatagttttaaaatcattttttacttttctcaaatttatcgaacaattaaaaactataaaaggagcaatttttcattttaattattttcttcattaattaaaaaattattttctctggaagaaaataaatataatagaaaaaaataaaaagaaaaggaaattacaaaaaaacaaaattaaataatatatattgatttatatattaattaaaaataatatatttttaaatttattatcaacaaaaaaaaaaaatttttaatcaaaaaatgaaggcttctattgctttaactgctattgccgctcttgctgctaacgcttctgctgcttgtttctctgaaagacttggttatccatgttgcagaggtaatgaagttttttacaccgataatgatggtgattggggtgttgaaaatggtaactggtgtggtattggtggtgcttctgctactacctgctggtctcaagctttaggttacccatgttgtacttctacttccgatgttgcctatgttgatggtgatggcaattggggtgttgaaaatggtaattggtgtggtattattgctggtggtaattcaagcaacaacaacagtggtagtaccattaatgttggtgatgttaccattggtaatcaatacactcacactggtaatccattcgctggtcacaaattcttcattaatccatactacactgctgaagtcgatggtgccatcgctcaaatttctaacgcttctcttagagctaaggctgaaaaaatgaaagaattctctaatgctatctggttagatactattaagaatatgaatgaatggttagaaaagaatcttaaatacgctcttgctgaacaaaatgaaactggtaagaccgttttaaccgttttcgttgtttacgatttaccaggtcgtgattgtcatgctcttgcttccaatggtgaacttcttgccaacgacagtgattgggctcgttaccaatcggaatacattgatgtcattgaagaaaaattaaagacttacaagagtcaaccagttgttcttgttgttgaaccagattctcttgctaacatggttactaatcttgattctactccagcttgtcgtgattctgaaaagtattacatggatggtcatgcttacttaattaaaaagcttggtgttcttccacatgttgctatgtaccttgatattggtcatgctttctggttaggatgggatgataaccgtttaaaggctggtaaggtttactccaaggttattcaatctggtgctccaggtaatgttcgtggtttcgcttctaacgttgctaactacactccatgggaagatccaactctttctcgtggtccagacactgaatggaatccatgtccagatgaaaagagatacattgaagccatgtacaaggacttcaagtctgctggtattaaatccgtttacttcattgatgatacttctcgtaatggtcacaaaaccgaccgtactcatccaggagaatggtgtaaccaaaccggagttggtattggtgctcgtccacaagccaatccaatctctggtatggactaccttgatgctttctactgggttaaaccactcggtgaatccgatggttactccgatactacagccgttcgttatgatggttattgtggtcatgctactgccatgaaaccagcaccagaagccggtcaatggttccaaaagcactttgaacaaggtcttgaaaatgctaatccaccactctaatcatattaacattaaataatatacattatatacatatagaaagaaacatgaatattantattaacataatcatacttnttaaataaattatt

SEQ ID NO: 6 - DNA, 1190 bp, Artificial Sequence
  Cochliobolus heterostrophus C4 cel7
ttaattaaaatgttgtctaacgtttttttgactgctgctttggctgctggtttggctcaagctttgccacaagctactccaactccaactgctgctccatctggtaatccatttgctggtaagaatttttacgctaacccatattattcttcagaagttcatactttggctatgccatctttgccagcttcattgaaaccagctgctactgctgttgctaaagttggttcttttgtttggatggatactatggctaaagttccattgatggatacttacttggctgatattaaagctaaaaatgctgctggtgctaatttgatgggtactttcgttgtttatgatttgccagatagagattgtgctgctttagcttctaatggtgaattgaaaattgatgaaggtggtgttgaaaaatacaagacacaatacattgataagattgctgctattatcaaaaagtacccagatgttaagattaatttggctattgaaccagattctttggctaatatggttactaatatgggtgttcaaaaatgttctagagctgctccatattacaaagaattgactgcttatgctttgaaaactttgaacttcaacaacgttgacatgtatatggatggtggtcatgctggttggttgggttgggatgctaatattggtccaactgctaaattgtttgctgaagtttacaaagctgctggttctccaagaggtgttagaggtattgttacaaacgtttctaattacaacgctttgagagtttcttcttgtccatctattactcaaggtaacaagaattgtgatgaagaaagatacattaatgctttggctccattgttgaaaaatgaaggttttccagctcattttattgttgatcaaggtagatcaggtaaagttccaactaatcaacaagaatggggtgattggtgtaatgtttctggtgctggttttggtactagaccaactactaatactggtaatgctttgattgatgctattgtttgggttaaaccaggtggtgaatctgatggtacttctgatacttctgctgcaagatatgatgctcattgtggtagaaattctgcttttaaaccagctccagaagctggtacttggtttcaagcttactttgaaatgttgttgaagaatgctaatccagctttggcattataaggcgcgcc

SEQ ID NO: 7 - DNA, 1394 bp, Artificial Sequence
  Gibberella zeae K59 cel6
ttaattaaaatgactgcttacaaattgtttttggctgctgcttttgctgctactgctttggctgctccagttgaagaaagacaatcttgttctaatggtgtttggtcacaatgtggtggtcaaaattggtctggtactccatgttgtacatctggtaacaagtgtgttaaggttaatgatttctactctcaatgtcaaccaggttctgctgatccatctccaacttctactattgtttctgctactactactaaagctactactacaggttctggtggttctgttacttctccaccaccagttgctacaaacaatccattttctggtgttgatttgtgggcaaacaattattacagatcagaagtttctactttggctattccaaaattgtctggtgctatggctactgctgctgcaaaagttgctgatgttccatcttttcaatggatggatacttacgatcatatttctttcatggaagattctttggctgatattagaaaagcaaacaaagcaggtggtaattatgctggtcaattcgttgtttatgatttgccagatagagattgtgctgctgctgcttctaatggtgaatactctttggataaagatggtaaaaacaagtacaaagcttatattgctgatcaaggtattttgcaagattactctgatactagaatcattttggttattgaaccagattctttagctaacatggttactaatatgaatgttccaaaatgtgctaatgctgcttctgcttacaaagaattgactattcatgctttgaaagaattgaatttgccaaacgtttcaatgtatattgatgctggtcatggtggttggttgggttggccagctaatttgccacctgctgctcaattgtatggtcaattgtacaaagatgctggtaaaccatctagattgagaggtttggttactaatgtttctaattacaacgcttggaaattatcttctaagccagattatactgaatctaacccaaattacgatgaacaaaagtacattcatgctttatctccattgttggaacaagaaggttggccaggcgctaagttcattgttgatcaaggtagatcaggtaaacaaccaactggtcaaaaagcttggggtgattggtgtaatgctccaggtactggttttggtttaagaccatctgctaatactggtgatgctttggttgatgcttttgtttgggttaaaccaggtggtgaatctgatggtacttctgatacttctgctgcaagatatgattatcattgtggtattgatggtgctgttaaaccagctccagaagctggtacttggtttcaagcttactttgaacaattgttgaagaatgctaatccatctttcttgttataaggcgcgcc

SEQ ID NO: 8 - DNA, 1379 bp, Artificial Sequence
  Irpex lacteus MC-2 cex3
ttaattaaaatgaagtctgctgcttttttggctgctttagctgctattttgccagcttacgttgctggtcaagctcaaacttgggctcaatgtggtggtattggttttactggtccaactacttgtgttgctggttctgtttgtactaaacaaaacgattactactctcaatgtattccaggttctgctactactccaacttctgctccaacatctgcaccaacttctcaaccatcacaaccatcttctacttcatctgctccatctggtccatcttctacaccaactccatctgctaacaatccatggactggttatcaaatttacttgtctccatactatgctaatgaagttgctgcagctgctaaagctattactgatccaactttggctgctaaagcagcttctgttgctaatattccaaatttcacttggttggattctgtttctaaaattgctgatttgaaaacttatttggctgatgcttctgctttgggtaaatcttctggtcaaaagcaattgttgcaaattgttgtttatgatttgccagatagagattgtgctgcaaaagcttctaatggtgaattttctattgctgataatggtttggctaactaccaaaactacattgatcaaattgttgctgctgttaaacaatttccagatgttagagttgttgctgttattgaaccagattctttggctaatttggttacaaatttaaacgttcaaaagtgtgctaatgctaaatctacttacttgactgctgttaattatgctttgaagcaattatcttctgttggtgtttatcaatatatggatgctggtcatgctggttggttgggttggccagctaatttaactccagctgctcaattgtttgctcaagtttattctgatgctggtaaatctccattcattaagggtttggctactaatgttgctaattacaatgctttgtctgctgcttctccagatccaattactcaaggtgatccaaattacgatgaaattcattacattaatgctttggctccagctttgcaatctgctggttttccagctacttttattgttgatcaaggtagatcaggtcaacaaaatcatagacaacaatggggtgattggtgtaacattaaaggtgctggttttggtactagaccaactactaatactggttcttctttgattgattctattgtttgggttaaaccaggtggtgaatctgatggtacttctaattcttcatctccaagatttgattctacttgttctttgtctgatgctactcaaccagctccagaagctggtacttggtttcaagcttactttgaaactttggtttctaaagctaatccaccattgttataaggcgcgcc

SEQ ID NO: 9 - DNA, 1349 bp, Artificial Sequence
  Volvariella volvacea cbhII-I
ttaattaaaatgtctagattctctgctttgactgctttgttgttgtctttgccattgttggctattgctcaatctccattgtatggtcaatgtggtggtaatggttggactggtccaaaaacttgtgtttctggtgctacttgtactgttattaatgattggtattggcaatgtttgccaggtaatggtccaacttcttcttctccaacttctactccaactacaactactactactggtggtccacaaccaactgttccagctgctggtaatccatatactggttacgaaatttacttgtctccatattatgctgctgaagctcaagctgctgctgctcaaatttctgatgctactcaaaaagctaaagctttgaaagttgctcaaattccaacttttacttggtttgatgttattgctaaaacttctactttgggtgattatttggctgaagcttctgctttgggtaaatcttctggtaaaaagtacttggttcaaattgttgtttatgatttgccagatagagattgtgctgctttggcttctaatggtgaattttctattgctaacaacggtttgaacaattacaaaggttacattgatcaattggttgcacaaattaagaaatacccagatgttagagttgttgctgttattgaaccagattctttggctaatttggttacaaatttgaacgtttctaagtgtgctaatgctcaaactgcttacaaagctggtgttacttatgctttgcaacaattgaactctgttggtgtttacatgtatttggatgctggtcatgctggttggttgggttggccagctaatttgaatccagctgctcaattgttttctcaattgtatagagatgctggttctccacaatacgttagaggtttggctactaatgttgctaattacaatgctttgtctgcttcttcaccagatccagttactcaaggtaatccaaattacgatgaattgcattacattaatgctttggctccagctttgcaatctggtggttttccagctcattttattgttgatcaaggtagatcaggtgttcaaaacattagacaacaatggggtgattggtgtaatgttaaaggtgctggttttggtcaaagaccaactttatctactggttcttctttgattgatgctattgtttggattaaaccaggtggtgaatgtgatggtactactaatacatcttctccaagatatgattctcattgtggtttgtctgatgctactccaaatgctcctgaagctggtcaatggtttcaagcttactttgaaactttggttagaaatgcttctccaccattgttataaggcgcgcc

SEQ ID NO: 10 - DNA, 1496 bp, Artificial Sequence
  Piromyces sp. E2 cel6A
ttaattaaaatgaaggcttctattgctttgactgctattgctgctttggctgctaatgcttctgctgcttgtttttctgaaagattgggttatccatgttgtagaggtaatgaagttttctacactgataatgatggtgattggggtgttgaaaatggtaattggtgtggtattggtggtgcttctgctactacttgttggtcacaagctttaggttacccttgttgtacttctacttctgatgttgcttacgttgatggtgacggtaactggggtgtcgaaaacggtaactggtgcggtataattgcaggtggtaattcttctaacaacaactctggttctactattaatgttggtgatgttactattggtaaccaatacactcatactggtaatccatttgctggtcataaattctttattaacccatactatactgctgaagttgatggtgctattgctcaaatttctaatgcttctttgagagctaaagctgaaaagatgaaagaattttctaacgctatttggttggatactattaagaatatgaacgaatggttggaaaagaatttgaaatatgctttggctgaacaaaatgaaactggtaagactgttttgacagtttttgttgtttatgatttgccaggtagagattgtcatgctttagcttctaatggtgaattgttggctaatgattctgattgggcaagatatcaatctgaatacattgatgttattgaagaaaagttgaaaacttacaagtctcaaccagttgttttggttgttgaaccagattctttggctaatatggttacaaatttggattctactccagcttgtagagattctgaaaaatactatatggatggtcatgcttacttgattaaaaagttgggtgttttgccacatgttgcaatgtatttggatattggtcatgctttttggttgggttgggatgataatagattgaaagctggtaaagtttactctaaggttattcaatctggtgctccaggtaatgttagaggttttgcttctaatgttgctaattatactccatgggaagatccaactttgtctagaggtccagatactgaatggaatccatgtccagatgaaaaaagatacattgaagcaatgtacaaagattttaagtctgctggtattaagtctgtttacttcattgatgatacttctagaaatggtcataagactgatagaactcatccaggtgaatggtgtaatcaaacaggtgttggtattggtgctagaccacaagctaatccaatttctggtatggattacttggatgctttttattgggttaaaccattgggtgaatctgatggttattctgatactactgctgtcagatatgatggttattgtggtcatgctactgctatgaaaccagctcctgaagctggtcaatggtttcaaaaacatttcgaacaaggtttggaaaatgctaatccaccattgttataaggcgcgcc

SEQ ID NO: 11 - protein, 389 aa, Cochliobolus heterostrophus
MLSNVFLTAALAAGLAQALPQATPTPTAAPSGNPFAGKNFYANPYYSSEVHTLAMPSLPASLKPAATAVAKVGSFVWMDTMAKVPLMDTYLADIKAKNAAGANLMGTFVVYDLPDRDCAALASNGELKIDEGGVEKYKTQYIDKIAAIIKKYPDVKINLAIEPDSLANMVTNMGVQKCSRAAPYYKELTAYALKTLNFNNVDMYMDGGHAGWLGWDANIGPTAKLFAEVYKAAGSPRGVRGIVTNVSNYNALRVSSCPSITQGNKNCDEERYINALAPLLKNEGFPAHFIVDQGRSGKVPTNQQEWGDWCNVSGAGFGTRPTTNTGNALIDAIVWVKPGGESDGTSDTSAARYDAHCGRNSAFKPAPEAGTWFQAYFEMLLKNANPALA

SEQ ID NO: 12 - protein, 457 aa, Gibberella zeae
MTAYKLFLAAAFAATALAAPVEERQSCSNGVWSQCGGQNWSGTPCCTSGNKCVKVNDFYSQCQPGSADPSPTSTIVSATTTKATTTGSGGSVTSPPPVATNNPFSGVDLWANNYYRSEVSTLAIPKLSGAMATAAAKVADVPSFQWMDTYDHISFMEDSLADIRKANKAGGNYAGQFVVYDLPDRDCAAAASNGEYSLDKDGKNKYKAYIADQGILQDYSDTRIILVIEPDSLANMVTNMNVPKCANAASAYKELTIHALKELNLPNVSMYIDAGHGGWLGWPANLPPAAQLYGQLYKDAGKPSRLRGLVTNVSNYNAWKLSSKPDYTESNPNYDEQKYIHALSPLLEQEGWPGAKFIVDQGRSGKQPTGQKAWGDWCNAPGTGFGLRPSANTGDALVDAFVWVKPGGESDGTSDTSAARYDYHCGIDGAVKPAPEAGTWFQAYFEQLLKNANPSFL

SEQ ID NO: 13 - protein, 457 aa, Irpex lacteus; identical to SEQ ID NO: 12

SEQ ID NO: 14 - protein, 442 aa, Volvariella volvacea
MSRFSALTALLLSLPLLAIAQSPLYGQCGGNGWTGPKTCVSGATCTVINDWYWQCLPGNGPTSSSPTSTPTTTTTTGGPQPTVPAAGNPYTGYEIYLSPYYAAEAQAAAAQISDATQKAKALKVAQIPTFTWFDVIAKTSTLGDYLAEASALGKSSGKKYLVQIVVYDLPDRDCAALASNGEFSIANNGLNNYKGYIDQLVAQIKKYPDVRVVAVIEPDSLANLVTNLNVSKCANAQTAYKAGVTYALQQLNSVGVYMYLDAGHAGWLGWPANLNPAAQLFSQLYRDAGSPQYVRGLATNVANYNALSASSPDPVTQGNPNYDELHYINALAPALQSGGFPAHFIVDQGRSGVQNIRQQWGDWCNVKGAGFGQRPTLSTGSSLIDAIVWIKPGGECDGTTNTSSPRYDSHCGLSDATPNAPEAGQWFQAYFETLVRNASPPL

SEQ ID NO: 15 - protein, 491 aa, Piromyces sp.
MKASIALTAIAALAANASAACFSERLGYPCCRGNEVFYTDNDGDWGVENGNWCGIGGASATTCWSQALGYPCCTSTSDVAYVDGDGNWGVENGNWCGIIAGGNSSNNNSGSTINVGDVTIGNQYTHTGNPFAGHKFFINPYYTAEVDGAIAQISNASLRAKAEKMKEFSNAIWLDTIKNMNEWLEKNLKYALAEQNETGKTVLTVFVVYDLPGRDCHALASNGELLANDSDWARYQSEYIDVIEEKLKTYKSQPVVLVVEPDSLANMVTNLDSTPACRDSEKYYMDGHAYLIKKLGVLPHVAMYLDIGHAFWLGWDDNRLKAGKVYSKVIQSGAPGNVRGFASNVANYTPWEDPTLSRGPDTEWNPCPDEKRYIEAMYKDFKSAGIKSVYFIDDTSRNGHKTDRTHPGEWCNQTGVGIGARPQANPISGMDYLDAFYWVKPLGESDGYSDTTAVRYDGYCGHATAMKPAPEAGQWFQKHFEQGLENANPPL

SEQ ID NO: 16 - protein, 19 aa, Artificial Sequence
  S. cerevisiae alpha mating factor signal sequence
MRFPSIFTAVLFAASSALA

SEQ ID NO: 17 - protein, 42 aa, Artificial Sequence
  optimized linker 1
GGGGSGGGGSAWHPQFGGENLYFQGDYKDDDKGGGGSGGGGS

SEQ ID NO: 18 - DNA, 144 bp, Artificial Sequence
  optimized linker 1
ggaggaggtggttcaggaggtggtgggtctgcttggcatccacaatttggaggaggcggtggtgaaaatctgtatttccagggaggcggaggtgattacaaggatgacgacaaaggaggtggtggatcaggaggtggtggctcc

SEQ ID NO: 19 - protein, 37 aa, Artificial Sequence
  optimized linker 2
GGGGSGGGGSWSHPQFEKGGENLYFQGGGGGSGGGGS

SEQ ID NO: 20 - DNA, 117 bp, Artificial Sequence
  optimized linker 2
ggtggcggtggatctggaggaggcggttcttggtctcacccacaatttgaaaagggtggagaaaacttgtactttcaaggcggtggtggaggttctggcggaggtggctccggctca

SEQ ID NO: 21 - DNA, 8452 bp, Artificial Sequence
  pMU784
agctgtttcctgtgtgaaattgttatccgctcacaattccacacaacatacgagccggaagcataaagtgtaaagcctggggtgcctaatgagtgaggtaactcacattaattgcgttgcgctcactgcccgctttccagtcgggaaacctgtcgtgccagctggattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataacgcatttaagcataaacacgcactatgccgttcttctcatgtatatatatatacaggcaacacgcagatataggtgcgacgtgaacagtgagctgtatgtgcgcagctcgcgttgcattttcggaagcgctcgttttcggaaacgctttgaagttcctattccgaagttcctattctctagctagaaagtataggaacttcagagcgcttttgaaaaccaaaagcgctctgaagacgcactttcaaaaaaccaaaaacgcaccggactgtaacgagctactaaaatattgcgaataccgcttccacaaacattgctcaaaagtatctctttgctatatatctctgtgctatatccctatataacctacccatccacctttcgctccttgaacttgcatctaaactcgacctctacattttttatgtttatctctagtattactctttagacaaaaaaattgtagtaagaactattcatagagtgaatcgaaaacaatacgaaaatgtaaacatttcctatacgtagtatatagagacaaaatagaagaaaccgttcataattttctgaccaatgaagaatcatcaacgctatcactttctgttcacaaagtatgcgcaatccacatcggtatagaatataatcggggatgcctttatcttgaaaaaatgcacccgcagcttcgctagtaatcagtaaacgcgggaagtggagtcaggctttttttatggaagagaaaatagacaccaaagtagccttcttctaaccttaacggacctacagtgcaaaaagttatcaagagactgcattatagagcgcacaaaggagaaaaaaagtaatctaagatgctttgttagaaaaatagcgctctcgggatgcatttttgtagaacaaaaaagaagtatagattctttgttggtaaaatagcgctctcgcgttgcatttctgttctgtaaaaatgcagctcagattctttgtttgaaaaattagcgctctcgcgttgcatttttgttttacaaaaatgaagcacagattcttcgttggtaaaatagcgctttcgcgttgcatttctgttctgtaaaaatgcagctcagattctttgtttgaaaaattagcgctctcgcgttgcatttttgttctacaaaatgaagcacagatgcttcgttaacaaagatatgctattgaagtgcaagatggaaacgcagaaaatgaaccggggatgcgacgtgcaagattacctatgcaatagatgcaatagtttctccaggaaccgaaatacatacattgtcttccgtaaagcgctagactatatattattatacaggttcaaatatactatctgtttcagggaaaactcccaggttcggatgttcaaaattcaatgatgggtaacaagagcttttcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaaccgaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggcgccattcgccattcaggctgcgcaactgttgggaagggcgatcggtgcgggcctcttcgctattacgccagctggcgaaggggggatgtgctgcaaggcgattaagttgggtaacgccagggttttcccagtcacgacgttgtaaaacgacggccagtgccaagctttctaactgatctatccaaaactgaaaattacattcttgattaggtttatcacaggcaaatgtaatttgtggtattttgccgttcaaaatctgtagaattttctcattggtcacattacaacctgaaaatactttatctacaatcataccattcttataacatgtccccttaatactaggatcaggcatgaacgcatcacagacaaaatcttcttgacaaacgtcacaattgatccctccccatccgttatcacaatgacaggtgtcattttgtgctcttatgggacgatccttattaccgctttcatccggtgatagaccgccacagaggggcagagagcaatcatcacctgcaaacccttctatacactcacatctaccagtgtacgaattgcattcagaaaactgtttgcattcaaaaataggtagcatacaattaaaacatggcgggcacgtatcattgcccttatcttgtgcagttagacgcgaatttttcgaagaagtaccttcaaagaatggggtctcatcttgttttgcaagtaccactgagcaggataataatagaaatgataatatactatagtagagataacgtcgatgacttcccatactgtaattgcttttagttgtgtatttttagtgtgcaagtttctgtaaatcgattaatttttttttctttcctctttttattaaccttaatttttattttagattcctgacttcaactcaagacgcacagatattataacatctgcacaataggcatttgcaagaattactcgtgagtaaggaaagagtgaggaactatcgcatacctgcatttaaagatgccgatttgggcgcgaatcctttattttggcttcaccctcatactattatcagggccagaaaaaggaagtgtttccctccttcttgaattgatgttaccctcataaagcacgtggcctcttatcgagaaagaaattaccgtcgctcgtgatttgtttgcaaaaagaacaaaactgaaaaaacccagacacgctcgacttcctgtcttcctattgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaacaaatataaaacttaattaaacaatggccaagaagttgttcattaccgctgccttagctgccgcagtgcttgctgcaccagtgatcgaagagagacaaaattgcggagccgtctggacacagtgcggaggcaacggctggcaaggcccaacatgttgtgcttctggctcaacgtgcgtggcacagaacgagtggtattcccagtgccttccaaactcccaggtgacttcttcaacaacccccagctcaacgtctacttcacagagatccacaagtacctcttctagcacaaccagaagtggctcatcctcatctagcagtacgacccctccacccgtatcaagtcctgtcacgagtatccctggcggagcaacctcaacagccagttattccggcaatcctttctctggagtgagattatttgcaaacgactattatagatcagaggttcacaaccttgcaattccttctatgacgggaaccctagccgcaaaggcttccgccgtagcagaagtccctagtttccaatggcttgacagaaacgttacaatagatacacttatggtacagactttatctcaggttagagctttgaataaggccggtgccaacccaccttatgctgcccaattagtagtctatgacttgccagatagagactgtgctgccgcagcttctaatggtgaattttccatcgcaaatggcggagctgcaaactatagatcatacattgatgcaataagaaaacacatcattgagtattctgatattagaataatccttgtgattgaaccagactccatggctaatatggttaccaacatgaatgtagccaagtgttctaacgcagcttccacataccatgagctaaccgtatatgcattaaaacaactgaatctacctaacgttgctatgtacttagatgccggtcatgccggatggttgggctggcctgcaaatatccaacccgcagctgaattgttcgctggaatctacaacgacgccggaaagcccgctgccgttagaggcttagccacaaatgttgcaaattacaacgcttggtcaattgctagtgccccttcttatacctcaccaaatcctaactacgatgagaaacattacatagaagcattttccccattgttaaactccgctggattccctgccagattcatcgtggataccggtagaaacggcaaacaaccaactggacaacaacaatggggagattggtgtaacgtcaagggaaccggcttcggcgtcaggcctacggcaaacaccggacacgagctagtcgacgcttttgtatgggttaagccaggtggcgaaagtgacggaacaagtgacacgagtgctgcaagatacgattaccactgtggtctgtccgacgctttacagcccgcccccgaggctggacaatggttccaggcttattttgaacaattgttaacgaacgcaaatccaccattctaaggcgcgccgaattcgagagactcgagactgaatcggatcgatcccgggcccgtcgagggatctgcgatagatcaatttttttcttttctctttccccatcctttacgctaaaataatagtttattttattttttgaatattttttatttatatacgtatatatagactattatttatcttttaatgattattaagatttttattaaaaaaaaattcgctcctcttttaatgcctttatgcagtttttttttcccattcgatatttctatgttcgggttcagcgtattttaagtttaataactcgaaaattctgcgttcgttaaagcttgcatgcctgcaggtcgactctagaggatccccgggtaccgagctcgaattaattcgtaatcatggtcat

SEQ ID NO: 22 - protein, 313 aa, Artificial Sequence
  Cochliobolus heterostrophus C4 cel7, GH Family 6 Domain (aa 42-354)
ANPYYSSEVHTLAMPSLPASLKPAATAVAKVGSFVWMDTMAKVPLMDTYLADIKAKNAAGANLMGTFVVYDLPDRDCAALASNGELKIDEGGVEKYKTQYIDKIAAIIKKYPDVKINLAIEPDSLANMVTNMGVQKCSRAAPYYKELTAYALKTLNFNNVDMYMDGGHAGWLGWDANIGPTAKLFAEVYKAAGSPRGVRGIVTNVSNYNALRVSSCPSITQGNKNCDEERYINALAPLLKNEGFPAHFIVDQGRSGKVPTNQQEWGDWCNVSGAGFGTRPTTNTGNALIDAIVWVKPGGESDGTSDTSAARYD

SEQ ID NO: 23 - protein, 18 aa, Artificial Sequence
  Cochliobolus heterostrophus C4 cel7, Signal Peptide (aa 1-18)
MLSNVFLTAALAAGLAQA

SEQ ID NO: 24 - protein, 313 aa, Artificial Sequence
  Gibberella zeae K59 cel6, GH Family 6 Domain (aa 111-423)
ANNYYRSEVSTLAIPKLSGAMATAAAKVADVPSFQWMDTYDHISFMEDSLADIRKANKAGGNYAGQFVVYDLPDRDCAAAASNGEYSLDKDGKNKYKAYIADQGILQDYSDTRIILVIEPDSLANMVTNMNVPKCANAASAYKELTIHALKELNLPNVSMYIDAGHGGWLGWPANLPPAAQLYGQLYKDAGKPSRLRGLVTNVSNYNAWKLSSKPDYTESNPNYDEQKYIHALSPLLEQEGWPGAKFIVDQGRSGKQPTGQKAWGDWCNAPGTGFGLRPSANTGDALVDAFVWVKPGGESDGTSDTSAARYDY

SEQ ID NO: 25 - protein, 18 aa, Artificial Sequence
  Gibberella zeae K59 cel6, Signal Peptide (aa 1-18)
MTAYKLFLAAAFAATALA

SEQ ID NO: 26 - protein, 29 aa, Artificial Sequence
  Gibberella zeae K59 cel6, CBM Domain (aa 31-59)
VWSQCGGQNWSGTPCCTSGNKCVKVNDFY

SEQ ID NO: 27 - protein, 313 aa, Artificial Sequence
  Irpex lacteus MC-2 cex3, GH Family 6 Domain (aa 107-419)
VDLWANNYYRSEVSTLAIPKLSGAMATAAAKVADVPSFQWMDTYDHISFMEDSLADIRKANKAGGNYAGQFVVYDLPDRDCAAAASNGEYSLDKDGKNKYKAYIADQGILQDYSDTRIILVIEPDSLANMVTNMNVPKCANAASAYKELTIHALKELNLPNVSMYIDAGHGGWLGWPANLPPAAQLYGQLYKDAGKPSRLRGLVTNVSNYNAWKLSSKPDYTESNPNYDEQKYIHALSPLLEQEGWPGAKFIVDQGRSGKQPTGQKAWGDWCNAPGTGFGLRPSANTGDALVDAFVWVKPGGESDGTSDTSAA

SEQ ID NO: 28 - protein, 20 aa, Artificial Sequence
  Irpex lacteus MC-2 cex3, Signal Peptide Domain (aa 1-20)
MTAYKLFLAAAFAATALAAP

SEQ ID NO: 29 - protein, 28 aa, Artificial Sequence
  Irpex lacteus MC-2 cex3, CBM Domain (aa 25-52)
QSCSNGVWSQCGGQNWSGTPCCTSGNKC

SEQ ID NO: 30 - protein, 290 aa, Artificial Sequence
  Volvariella volvacea cbhII-I, GH Family 6 Domain (aa 120-409)
KALKVAQIPTFTWFDVIAKTSTLGDYLAEASALGKSSGKKYLVQIVVYDLPDRDCAALASNGEFSIANNGLNNYKGYIDQLVAQIKKYPDVRVVAVIEPDSLANLVTNLNVSKCANAQTAYKAGVTYALQQLNSVGVYMYLDAGHAGWLGWPANLNPAAQLFSQLYRDAGSPQYVRGLATNVANYNALSASSPDPVTQGNPNYDELHYINALAPALQSGGFPAHFIVDQGRSGVQNIRQQWGDWCNVKGAGFGQRPTLSTGSSLIDAIVWIKPGGECDGTTNTSSPRYDS

SEQ ID NO: 31 - protein, 20 aa, Artificial Sequence
  Volvariella volvacea cbhII-I, Signal Peptide Domain (aa 1-20)
MSRFSALTALLLSLPLLAIA

SEQ ID NO: 32 - protein, 28 aa, Artificial Sequence
  Volvariella volvacea cbhII-I, CBM Domain (aa 25-52)
YGQCGGNGWTGPKTCVSGATCTVINDWY

SEQ ID NO: 33 - protein, 320 aa, Artificial Sequence
  Piromyces sp. E2 cel6A, GH Family 6 Domain (aa 138-457)
INPYYTAEVDGAIAQISNASLRAKAEKMKEFSNAIWLDTIKNMNEWLEKNLKYALAEQNETGKTVLTVFVVYDLPGRDCHALASNGELLANDSDWARYQSEYIDVIEEKLKTYKSQPVVLVVEPDSLANMVTNLDSTPACRDSEKYYMDGHAYLIKKLGVLPHVAMYLDIGHAFWLGWDDNRLKAGKVYSKVIQSGAPGNVRGFASNVANYTPWEDPTLSRGPDTEWNPCPDEKRYIEAMYKDFKSAGIKSVYFIDDTSRNGHKTDRTHPGEWCNQTGVGIGARPQANPISGMDYLDAFYWVKPLGESDGYSDTTAVRYD

SEQ ID NO: 34 - protein, 19 aa, Artificial Sequence
  Piromyces sp. E2 cel6A, Signal Peptide Domain (aa 1-19)
MKASIALTAIAALAANASA

SEQ ID NO: 35 - protein, 35 aa, Artificial Sequence
  Piromyces sp. E2 cel6A, CBM Domain (aa 21-55)
CFSERLGYPCCRGNEVFYTDNDGDWGVENGNWCGI

SEQ ID NO: 36 - protein, 37 aa, Artificial Sequence
  Piromyces sp. E2 cel6A, CBM Domain (aa 62-98)
TCWSQALGYPCCTSTSDVAYVDGDGNWGVENGNWCGI

SEQ ID NO: 37 - DNA, 8174 bp, Artificial Sequence
  pRDH150
agctgtttcctgtgtgaaattgttatccgctcacaattccacacaacatacgagccggaagcataaagtgtaaagcctggggtgcctaatgagtgaggtaactcacattaattgcgttgcgctcactgcccgctttccagtcgggaaacctgtcgtgccagctggattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataacgcatttaagcataaacacgcactatgccgttcttctcatgtatatatatatacaggcaacacgcagatataggtgcgacgtgaacagtgagctgtatgtgcgcagctcgcgttgcattttcggaagcgctcgttttcggaaacgctttgaagttcctattccgaagttcctattctctagctagaaagtataggaacttcagagcgcttttgaaaaccaaaagcgctctgaagacgcactttcaaaaaaccaaaaacgcaccggactgtaacgagctactaaaatattgcgaataccgcttccacaaacattgctcaaaagtatctctttgctatatatctctgtgctatatccctatataacctacccatccacctttcgctccttgaacttgcatctaaactcgacctctacattttttatgtttatctctagtattactctttagacaaaaaaattgtagtaagaactattcatagagtgaatcgaaaacaatacgaaaatgtaaacatttcctatacgtagtatatagagacaaaatagaagaaaccgttcataattttctgaccaatgaagaatcatcaacgctatcactttctgttcacaaagtatgcgcaatccacatcggtatagaatataatcggggatgcctttatcttgaaaaaatgcacccgcagcttcgctagtaatcagtaaacgcgggaagtggagtcaggctttttttatggaagagaaaatagacaccaaagtagccttcttctaaccttaacggacctacagtgcaaaaagttatcaagagactgcattatagagcgcacaaaggagaaaaaaagtaatctaagatgctttgttagaaaaatagcgctctcgggatgcatttttgtagaacaaaaaagaagtatagattctttgttggtaaaatagcgctctcgcgttgcatttctgttctgtaaaaatgcagctcagattctttgtttgaaaaattagcgctctcgcgttgcatttttgttttacaaaaatgaagcacagattcttcgttggtaaaatagcgctttcgcgttgcatttctgttctgtaaaaatgcagctcagattctttgtttgaaaaattagcgctctcgcgttgcatttttgttctacaaaatgaagcacagatgcttcgttaacaaagatatgctattgaagtgcaagatggaaacgcagaaaatgaaccggggatgcgacgtgcaagattacctatgcaatagatgcaatagtttctccaggaaccgaaatacatacattgtcttccgtaaagcgctagactatatattattatacaggttcaaatatactatctgtttcagggaaaactcccaggttcggatgttcaaaattcaatgatgggtaacaagagcttttcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaaccgaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggcgccattcgccattcaggctgcgcaactgttgggaagggcgatcggtgcgggcctcttcgctattacgccagctggcgaaggggggatgtgctgcaaggcgattaagttgggtaacgccagggttttcccagtcacgacgttgtaaaacgacggccagtgccaagctttctaactgatctatccaaaactgaaaattacattcttgattaggtttatcacaggcaaatgtaatttgtggtattttgccgttcaaaatctgtagaattttctcattggtcacattacaacctgaaaatactttatctacaatcataccattcttataacatgtccccttaatactaggatcaggcatgaacgcatcacagacaaaatcttcttgacaaacgtcacaattgatccctccccatccgttatcacaatgacaggtgtcattttgtgctcttatgggacgatccttattaccgctttcatccggtgatagaccgccacagaggggcagagagcaatcatcacctgcaaacccttctatacactcacatctaccagtgtacgaattgcattcagaaaactgtttgcattcaaaaataggtagcatacaattaaaacatggcgggcacgtatcattgcccttatcttgtgcagttagacgcgaatttttcgaagaagtaccttcaaagaatggggtctcatcttgttttgcaagtaccactgagcaggataataatagaaatgataatatactatagtagagataacgtcgatgacttcccatactgtaattgcttttagttgtgtatttttagtgtgcaagtttctgtaaatcgattaatttttttttctttcctctttttattaaccttaatttttattttagattcctgacttcaactcaagacgcacagatattataacatctgcacaataggcatttgcaagaattactcgtgagtaaggaaagagtgaggaactatcgcatacctgcatttaaagatgccgatttgggcgcgaatcctttattttggcttcaccctcatactattatcagggccagaaaaaggaagtgtttccctccttcttgaattgatgttaccctcataaagcacgtggcctcttatcgagaaagaaattaccgtcgctcgtgatttgtttgcaaaaagaacaaaactgaaaaaacccagacacgctcgacttcctgtcttcctattgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaacaaatataaaacttaattaaaatgttgtctaacgtttttttgactgctgctttggctgctggtttggctcaagctttgccacaagctactccaactccaactgctgctccatctggtaatccatttgctggtaagaatttttacgctaacccatattattcttcagaagttcatactttggctatgccatctttgccagcttcattgaaaccagctgctactgctgttgctaaagttggttcttttgtttggatggatactatggctaaagttccattgatggatacttacttggctgatattaaagctaaaaatgctgctggtgctaatttgatgggtactttcgttgtttatgatttgccagatagagattgtgctgctttagcttctaatggtgaattgaaaattgatgaaggtggtgttgaaaaatacaagacacaatacattgataagattgctgctattatcaaaaagtacccagatgttaagattaatttggctattgaaccagattctttggctaatatggttactaatatgggtgttcaaaaatgttctagagctgctccatattacaaagaattgactgcttatgctttgaaaactttgaacttcaacaacgttgacatgtatatggatggtggtcatgctggttggttgggttgggatgctaatattggtccaactgctaaattgtttgctgaagtttacaaagctgctggttctccaagaggtgttagaggtattgttacaaacgtttctaattacaacgctttgagagtttcttcttgtccatctattactcaaggtaacaagaattgtgatgaagaaagatacattaatgctttggctccattgttgaaaaatgaaggttttccagctcattttattgttgatcaaggtagatcaggtaaagttccaactaatcaacaagaatggggtgattggtgtaatgtttctggtgctggttttggtactagaccaactactaatactggtaatgctttgattgatgctattgtttgggttaaaccaggtggtgaatctgatggtacttctgatacttctgctgcaagatatgatgctcattgtggtagaaattctgcttttaaaccagctccagaagctggtacttggtttcaagcttactttgaaatgttgttgaagaatgctaatccagctttggcattataaggcgcgccgaattcgagagactcgagactgaatcggatcgatcccgggcccgtcgagggatctgcgatagatcaatttttttcttttctctttccccatcctttacgctaaaataatagtttattttattttttgaatattttttatttatatacgtatatatagactattatttatcttttaatgattattaagatttttattaaaaaaaaattcgctcctcttttaatgcctttatgcagtttttttttcccattcgatatttctatgttcgggttcagcgtattttaagtttaataactcgaaaattctgcgttcgttaaagcttgcatgcctgcaggtcgactctagaggatccccgggtaccgagctcgaattaattcgtaatcatggtcat

SEQ ID NO: 38 - DNA, 8378 bp, Artificial Sequence
  pRDH151
agctgtttcctgtgtgaaattgttatccgctcacaattccacacaacatacgagccggaagcataaagtgtaaagcctggggtgcctaatgagtgaggtaactcacattaattgcgttgcgctcactgcccgctttccagtcgggaaacctgtcgtgccagctggattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataacgcatttaagcataaacacgcactatgccgttcttctcatgtatatatatatacaggcaacacgcagatataggtgcgacgtgaacagtgagctgtatgtgcgcagctcgcgttgcattttcggaagcgctcgttttcggaaacgctttgaagttcctattccgaagttcctattctctagctagaaagtataggaacttcagagcgcttttgaaaaccaaaagcgctctgaagacgcactttcaaaaaaccaaaaacgcaccggactgtaacgagctactaaaatattgcgaataccgcttccacaaacattgctcaaaagtatctctttgctatatatctctgtgctatatccctatataacctacccatccacctttcgctccttgaacttgcatctaaactcgacctctacattttttatgtttatctctagtattactctttagacaaaaaaattgtagtaagaactattcatagagtgaatcgaaaacaatacgaaaatgtaaacatttcctatacgtagtatatagagacaaaatagaagaaaccgttcataattttctgaccaatgaagaatcatcaacgctatcactttctgttcacaaagtatgcgcaatccacatcggtatagaatataatcggggatgcctttatcttgaaaaaatgcacccgcagcttcgctagtaatcagtaaacgcgggaagtggagtcaggctttttttatggaagagaaaatagacaccaaagtagccttcttctaaccttaacggacctacagtgcaaaaagttatcaagagactgcattatagagcgcacaaaggagaaaaaaagtaatctaagatgctttgttagaaaaatagcgctctcgggatgcatttttgtagaacaaaaaagaagtatagattctttgttggtaaaatagcgctctcgcgttgcatttctgttctgtaaaaatgcagctcagattctttgtttgaaaaattagcgctctcgcgttgcatttttgttttacaaaaatgaagcacagattcttcgttggtaaaatagcgctttcgcgttgcatttctgttctgtaaaaatgcagctcagattctttgtttgaaaaattagcgctctcgcgttgcatttttgttctacaaaatgaagcacagatgcttcgttaacaaagatatgctattgaagtgcaagatggaaacgcagaaaatgaaccggggatgcgacgtgcaagattacctatgcaatagatgcaatagtttctccaggaaccgaaatacatacattgtcttccgtaaagcgctagactatatattattatacaggttcaaatatactatctgtttcagggaaaactcccaggttcggatgttcaaaattcaatgatgggtaacaagagcttttcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaaccgaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggcgccattcgccattcaggctgcgcaactgttgggaagggcgatcggtgcgggcctcttcgctattacgccagctggcgaaggggggatgtgctgcaaggcgattaagttgggtaacgccagggttttcccagtcacgacgttgtaaaacgacggccagtgccaagctttctaactgatctatccaaaactgaaaattacattcttgattaggtttatcacaggcaaatgtaatttgtggtattttgccgttcaaaatctgtagaattttctcattggtcacattacaacctgaaaatactttatctacaatcataccattcttataacatgtccccttaatactaggatcaggcatgaacgcatcacagacaaaatcttcttgacaaacgtcacaattgatccctccccatccgttatcacaatgacaggtgtcattttgtgctcttatgggacgatccttattaccgctttcatccggtgatagaccgccacagaggggcagagagcaatcatcacctgcaaacccttctatacactcacatctaccagtgtacgaattgcattcagaaaactgtttgcattcaaaaataggtagcatacaattaaaacatggcgggcacgtatcattgcccttatcttgtgcagttagacgcgaatttttcgaagaagtaccttcaaagaatggggtctcatcttgttttgcaagtaccactgagcaggataataatagaaatgataatatactatagtagagataacgtcgatgacttcccatactgtaattgcttttagttgtgtatttttagtgtgcaagtttctgtaaatcgattaatttttttttctttcctctttttattaaccttaatttttattttagattcctgacttcaactcaagacgcacagatattataacatctgcacaataggcatttgcaagaattactcgtgagtaaggaaagagtgaggaactatcgcatacctgcatttaaagatgccgatttgggcgcgaatcctttattttggcttcaccctcatactattatcagggccagaaaaaggaagtgtttccctccttcttgaattgatgttaccctcataaagcacgtggcctcttatcgagaaagaaattaccgtcgctcgtgatttgtttgcaaaaagaacaaaactgaaaaaacccagacacgctcgacttcctgtcttcctattgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaacaaatataaaacttaattaaaatgactgcttacaaattgtttttggctgctgcttttgctgctactgctttggctgctccagttgaagaaagacaatcttgttctaatggtgtttggtcacaatgtggtggtcaaaattggtctggtactccatgttgtacatctggtaacaagtgtgttaaggttaatgatttctactctcaatgtcaaccaggttctgctgatccatctccaacttctactattgtttctgctactactactaaagctactactacaggttctggtggttctgttacttctccaccaccagttgctacaaacaatccattttctggtgttgatttgtgggcaaacaattattacagatcagaagtttctactttggctattccaaaattgtctggtgctatggctactgctgctgcaaaagttgctgatgttccatcttttcaatggatggatacttacgatcatatttctttcatggaagattctttggctgatattagaaaagcaaacaaagcaggtggtaattatgctggtcaattcgttgtttatgatttgccagatagagattgtgctgctgctgcttctaatggtgaatactctttggataaagatggtaaaaacaagtacaaagcttatattgctgatcaaggtattttgcaagattactctgatactagaatcattttggttattgaaccagattctttagctaacatggttactaatatgaatgttccaaaatgtgctaatgctgcttctgcttacaaagaattgactattcatgctttgaaagaattgaatttgccaaacgtttcaatgtatattgatgctggtcatggtggttggttgggttggccagctaatttgccacctgctgctcaattgtatggtcaattgtacaaagatgctggtaaaccatctagattgagaggtttggttactaatgtttctaattacaacgcttggaaattatcttctaagccagattatactgaatctaacccaaattacgatgaacaaaagtacattcatgctttatctccattgttggaacaagaaggttggccaggcgctaagttcattgttgatcaaggtagatcaggtaaacaaccaactggtcaaaaagcttggggtgattggtgtaatgctccaggtactggttttggtttaagaccatctgctaatactggtgatgctttggttgatgcttttgtttgggttaaaccaggtggtgaatctgatggtacttctgatacttctgctgcaagatatgattatcattgtggtattgatggtgctgttaaaccagctccagaagctggtacttggtttcaagcttactttgaacaattgttgaagaatgctaatccatctttcttgttataaggcgcgccgaattcgagagactcgagactgaatcggatcgatcccgggcccgtcgagggatctgcgatagatcaatttttttcttttctctttccccatcctttacgctaaaataatagtttattttattttttgaatattttttatttatatacgtatatatagactattatttatcttttaatgattattaagatttttattaaaaaaaaattcgctcctcttttaatgcctttatgcagtttttttttcccattcgatatttctatgttcgggttcagcgtattttaagtttaataactcgaaaattctgcgttcgttaaagcttgcatgcctgcaggtcgactctagaggatccccgggtaccgagctcgaattaattcgtaatcatggtcat

SEQ ID NO: 39 - DNA, 8363 bp, Artificial Sequence
  pRDH152
agctgtttcctgtgtgaaattgttatccgctcacaattccacacaacatacgagccggaagcataaagtgtaaagcctggggtgcctaatgagtgaggtaactcacattaattgcgttgcgctcactgcccgctttccagtcgggaaacctgtcgtgccagctggattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataacgcatttaagcataaacacgcactatgccgttcttctcatgtatatatatatacaggcaacacgcagatataggtgcgacgtgaacagtgagctgtatgtgcgcagctcgcgttgcattttcggaagcgctcgttttcggaaacgctttgaagttcctattccgaagttcctattctctagctagaaagtataggaacttcagagcgcttttgaaaaccaaaagcgctctgaagacgcactttcaaaaaaccaaaaacgcaccggactgtaacgagctactaaaatattgcgaataccgcttccacaaacattgctcaaaagtatctctttgctatatatctctgtgctatatccctatataacctacccatccacctttcgctccttgaacttgcatctaaactcgacctctacattttttatgtttatctctagtattactctttagacaaaaaaattgtagtaagaactattcatagagtgaatcgaaaacaatacgaaaatgtaaacatttcctatacgtagtatatagagacaaaatagaagaaaccgttcataattttctgaccaatgaagaatcatcaacgctatcactttctgttcacaaagtatgcgcaatccacatcggtatagaatataatcggggatgcctttatcttgaaaaaatgcacccgcagcttcgctagtaatcagtaaacgcgggaagtggagtcaggctttttttatggaagagaaaatagacaccaaagtagccttcttctaaccttaacggacctacagtgcaaaaagttatcaagagactgcattatagagcgcacaaaggagaaaaaaagtaatctaagatgctttgttagaaaaatagcgctctcgggatgcatttttgtagaacaaaaaagaagtatagattctttgttggtaaaatagcgctctcgcgttgcatttctgttctgtaaaaatgcagctcagattctttgtttgaaaaattagcgctctcgcgttgcatttttgttttacaaaaatgaagcacagattcttcgttggtaaaatagcgctttcgcgttgcatttctgttctgtaaaaatgcagctcagattctttgtttgaaaaattagcgctctcgcgttgcatttttgttctacaaaatgaagcacagatgcttcgttaacaaagatatgctattgaagtgcaagatggaaacgcagaaaatgaaccggggatgcgacgtgcaagattacctatgcaatagatgcaatagtttctccaggaaccgaaatacatacattgtcttccgtaaagcgctagactatatattattatacaggttcaaatatactatctgtttcagggaaaactcccaggttcggatgttcaaaattcaatgatgggtaacaagagcttttcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaaccgaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggcgccattcgccattcaggctgcgcaactgttgggaagggcgatcggtgcgggcctcttcgctattacgccagctggcgaaggggggatgtgctgcaaggcgattaagttgggtaacgccagggttttcccagtcacgacgttgtaaaacgacggccagtgccaagctttctaactgatctatccaaaactgaaaattacattcttgattaggtttatcacaggcaaatgtaatttgtggtattttgccgttcaaaatctgtagaattttctcattggtcacattacaacctgaaaatactttatctacaatcataccattcttataacatgtccccttaatactaggatcaggcatgaacgcatcacagacaaaatcttcttgacaaacgtcacaattgatccctccccatccgttatcacaatgacaggtgtcattttgtgctcttatgggacgatccttattaccgctttcatccggtgatagaccgccacagaggggcagagagcaatcatcacctgcaaacccttctatacactcacatctaccagtgtacgaattgcattcagaaaactgtttgcattcaaaaataggtagcatacaattaaaacatggcgggcacgtatcattgcccttatcttgtgcagttagacgcgaatttttcgaagaagtaccttcaaagaatggggtctcatcttgttttgcaagtaccactgagcaggataataatagaaatgataatatactatagtagagataacgtcgatgacttcccatactgtaattgcttttagttgtgtatttttagtgtgcaagtttctgtaaatcgattaatttttttttctttcctctttttattaaccttaatttttattttagattcctgacttcaactcaagacgcacagatattataacatctgcacaataggcatttgcaagaattactcgtgagtaaggaaagagtgaggaactatcgcatacctgcatttaaagatgccgatttgggcgcgaatcctttattttggcttcaccctcatactattatcagggccagaaaaaggaagtgtttccctccttcttgaattgatgttaccctcataaagcacgtggcctcttatcgagaaagaaattaccgtcgctcgtgatttgtttgcaaaaagaacaaaactgaaaaaacccagacacgctcgacttcctgtcttcctattgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaacaaatataaaacttaattaaaatgaagtctgctgcttttttggctgctttagctgctattttgccagcttacgttgctggtcaagctcaaacttgggctcaatgtggtggtattggttttactggtccaactacttgtgttgctggttctgtttgtactaaacaaaacgattactactctcaatgtattccaggttctgctactactccaacttctgctccaacatctgcaccaacttctcaaccatcacaaccatcttctacttcatctgctccatctggtccatcttctacaccaactccatctgctaacaatccatggactggttatcaaatttacttgtctccatactatgctaatgaagttgctgcagctgctaaagctattactgatccaactttggctgctaaagcagcttctgttgctaatattccaaatttcacttggttggattctgtttctaaaattgctgatttgaaaacttatttggctgatgcttctgctttgggtaaatcttctggtcaaaagcaattgttgcaaattgttgtttatgatttgccagatagagattgtgctgcaaaagcttctaatggtgaattttctattgctgataatggtttggctaactaccaaaactacattgatcaaattgttgctgctgttaaacaatttccagatgttagagttgttgctgttattgaaccagattctttggctaatttggttacaaatttaaacgttcaaaagtgtgctaatgctaaatctacttacttgactgctgttaattatgctttgaagcaattatcttctgttggtgtttatcaatatatggatgctggtcatgctggttggttgggttggccagctaatttaactccagctgctcaattgtttgctcaagtttattctgatgctggtaaatctccattcattaagggtttggctactaatgttgctaattacaatgctttgtctgctgcttctccagatccaattactcaaggtgatccaaattacgatgaaattcattacattaatgctttggctccagctttgcaatctgctggttttccagctacttttattgttgatcaaggtagatcaggtcaacaaaatcatagacaacaatggggtgattggtgtaacattaaaggtgctggttttggtactagaccaactactaatactggttcttctttgattgattctattgtttgggttaaaccaggtggtgaatctgatggtacttctaattcttcatctccaagatttgattctacttgttctttgtctgatgctactcaaccagctccagaagctggtacttggtttcaagcttactttgaaactttggtttctaaagctaatccaccattgttataaggcgcgccgaattcgagagactcgagactgaatcggatcgatcccgggcccgtcgagggatctgcgatagatcaatttttttcttttctctttccccatcctttacgctaaaataatagtttattttattttttgaatattttttatttatatacgtatatatagactattatttatcttttaatgattattaagatttttattaaaaaaaaattcgctcctcttttaatgcctttatgcagtttttttttcccattcgatatttctatgttcgggttcagcgtattttaagtttaataactcgaaaattctgcgttcgttaaagcttgcatgcctgcaggtcgactctagaggatccccgggtaccgagctcgaattaattcgtaatcatggtcat

SEQ ID NO: 40 - DNA, 8333 bp, Artificial Sequence
  pRDH153
agctgtttcctgtgtgaaattgttatccgctcacaattccacacaacatacgagccggaagcataaagtgtaaagcctggggtgcctaatgagtgaggtaactcacattaattgcgttgcgctcactgcccgctttccagtcgggaaacctgtcgtgccagctggattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataacgcatttaagcataaacacgcactatgccgttcttctcatgtatatatatatacaggcaacacgcagatataggtgcgacgtgaacagtgagctgtatgtgcgcagctcgcgttgcattttcggaagcgctcgttttcggaaacgctttgaagttcctattccgaagttcctattctctagctagaaagtataggaacttcagagcgcttttgaaaaccaaaagcgctctgaagacgcactttcaaaaaaccaaaaacgcaccggactgtaacgagctactaaaatattgcgaataccgcttccacaaacattgctcaaaagtatctctttgctatatatctctgtgctatatccctatataacctacccatccacctttcgctccttgaacttgcatctaaactcgacctctacattttttatgtttatctctagtattactctttagacaaaaaaattgtagtaagaactattcatagagtgaatcgaaaacaatacgaaaatgtaaacatttcctatacgtagtatatagagacaaaatagaagaaaccgttcataattttctgaccaatgaagaatcatcaacgctatcactttctgttcacaaagtatgcgcaatccacatcggtatagaatataatcggggatgcctttatcttgaaaaaatgcacccgcagcttcgctagtaatcagtaaacgcgggaagtggagtcaggctttttttatggaagagaaaatagacaccaaagtagccttcttctaaccttaacggacctacagtgcaaaaagttatcaagagactgcattatagagcgcacaaaggagaaaaaaagtaatctaagatgctttgttagaaaaatagcgctctcgggatgcatttttgtagaacaaaaaagaagtatagattctttgttggtaaaatagcgctctcgcgttgcatttctgttctgtaaaaatgcagctcagattctttgtttgaaaaattagcgctctcgcgttgcatttttgttttacaaaaatgaagcacagattcttcgttggtaaaatagcgctttcgcgttgcatttctgttctgtaaaaatgcagctcagattctttgtttgaaaaattagcgctctcgcgttgcatttttgttctacaaaatgaagcacagatgcttcgttaacaaagatatgctattgaagtgcaagatggaaacgcagaaaatgaaccggggatgcgacgtgcaagattacctatgcaatagatgcaatagtttctccaggaaccgaaatacatacattgtcttccgtaaagcgctagactatatattattatacaggttcaaatatactatctgtttcagggaaaactcccaggttcggatgttcaaaattcaatgatgggtaacaagagcttttcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaaccgaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggcgccattcgccattcaggctgcgcaactgttgggaagggcgatcggtgcgggcctcttcgctattacgccagctggcgaaggggggatgtgctgcaaggcgattaagttgggtaacgccagggttttcccagtcacgacgttgtaaaacgacggccagtgccaagctttctaactgatctatccaaaactgaaaattacattcttgattaggtttatcacaggcaaatgtaatttgtggtattttgccgttcaaaatctgtagaattttctcattggtcacattacaacctgaaaatactttatctacaatcataccattcttataacatgtccccttaatactaggatcaggcatgaacgcatcacagacaaaatcttcttgacaaacgtcacaattgatccctccccatccgttatcacaatgacaggtgtcattttgtgctcttatgggacgatccttattaccgctttcatccggtgatagaccgccacagaggggcagagagcaatcatcacctgcaaacccttctatacactcacatctaccagtgtacgaattgcattcagaaaactgtttgcattcaaaaataggtagcatacaattaaaacatggcgggcacgtatcattgcccttatcttgtgcagttagacgcgaatttttcgaagaagtaccttcaaagaatggggtctcatcttgttttgcaagtaccactgagcaggataataatagaaatgataatatactatagtagagataacgtcgatgacttcccatactgtaattgcttttagttgtgtatttttagtgtgcaagtttctgtaaatcgattaatttttttttctttcctctttttattaaccttaatttttattttagattcctgacttcaactcaagacgcacagatattataacatctgcacaataggcatttgcaagaattactcgtgagtaaggaaagagtgaggaactatcgcatacctgcatttaaagatgccgatttgggcgcgaatcctttattttggcttcaccctcatactattatcagggccagaaaaaggaagtgtttccctccttcttgaattgatgttaccctcataaagcacgtggcctcttatcgagaaagaaattaccgtcgctcgtgatttgtttgcaaaaagaacaaaactgaaaaaacccagacacgctcgacttcctgtcttcctattgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaacaaatataaaacttaattaaaatgtctagattctctgctttgactgctttgttgttgtctttgccattgttggctattgctcaatctccattgtatggtcaatgtggtggtaatggttggactggtccaaaaacttgtgtttctggtgctacttgtactgttattaatgattggtattggcaatgtttgccaggtaatggtccaacttcttcttctccaacttctactccaactacaactactactactggtggtccacaaccaactgttccagctgctggtaatccatatactggttacgaaatttacttgtctccatattatgctgctgaagctcaagctgctgctgctcaaatttctgatgctactcaaaaagctaaagctttgaaagttgctcaaattccaacttttacttggtttgatgttattgctaaaacttctactttgggtgattatttggctgaagcttctgctttgggtaaatcttctggtaaaaagtacttggttcaaattgttgtttatgatttgccagatagagattgtgctgctttggcttctaatggtgaattttctattgctaacaacggtttgaacaattacaaaggttacattgatcaattggttgcacaaattaagaaatacccagatgttagagttgttgctgttattgaaccagattctttggctaatttggttacaaatttgaacgtttctaagtgtgctaatgctcaaactgcttacaaagctggtgttacttatgctttgcaacaattgaactctgttggtgtttacatgtatttggatgctggtcatgctggttggttgggttggccagctaatttgaatccagctgctcaattgttttctcaattgtatagagatgctggttctccacaatacgttagaggtttggctactaatgttgctaattacaatgctttgtctgcttcttcaccagatccagttactcaaggtaatccaaattacgatgaattgcattacattaatgctttggctccagctttgcaatctggtggttttccagctcattttattgttgatcaaggtagatcaggtgttcaaaacattagacaacaatggggtgattggtgtaatgttaaaggtgctggttttggtcaaagaccaactttatctactggttcttctttgattgatgctattgtttggattaaaccaggtggtgaatgtgatggtactactaatacatcttctccaagatatgattctcattgtggtttgtctgatgctactccaaatgctcctgaagctggtcaatggtttcaagcttactttgaaactttggttagaaatgcttctccaccattgttataaggcgcgccgaattcgagagactcgagactgaatcggatcgatcccgggcccgtcgagggatctgcgatagatcaatttttttcttttctctttccccatcctttacgctaaaataatagtttattttattttttgaatattttttatttatatacgtatatatagactattatttatcttttaatgattattaagatttttattaaaaaaaaattcgctcctcttttaatgcctttatgcagtttttttttcccattcgatatttctatgttcgggttcagcgtattttaagtttaataactcgaaaattctgcgttcgttaaagcttgcatgcctgcaggtcgactctagaggatccccgggtaccgagctcgaattaattcgtaatcatggtcat

SEQ ID NO: 41 - DNA, 8480 bp, Artificial Sequence
  pRDH154
agctgtttcctgtgtgaaattgttatccgctcacaattccacacaacatacgagccggaagcataaagtgtaaagcctggggtgcctaatgagtgaggtaactcacattaattgcgttgcgctcactgcccgctttccagtcgggaaacctgtcgtgccagctggattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataacgcatttaagcataaacacgcactatgccgttcttctcatgtatatatatatacaggcaacacgcagatataggtgcgacgtgaacagtgagctgtatgtgcgcagctcgcgttgcattttcggaagcgctcgttttcggaaacgctttgaagttcctattccgaagttcctattctctagctagaaagtataggaacttcagagcgcttttgaaaaccaaaagcgctctgaagacgcactttcaaaaaaccaaaaacgcaccggactgtaacgagctactaaaatattgcgaataccgcttccacaaacattgctcaaaagtatctctttgctatatatctctgtgctatatccctatataacctacccatccacctttcgctccttgaacttgcatctaaactcgacctctacattttttatgtttatctctagtattactctttagacaaaaaaattgtagtaagaactattcatagagtgaatcgaaaacaatacgaaaatgtaaacatttcctatacgtagtatatagagacaaaatagaagaaaccgttcataattttctgaccaatgaagaatcatcaacgctatcactttctgttcacaaagtatgcgcaatccacatcggtatagaatataatcggggatgcctttatcttgaaaaaatgcacccgcagcttcgctagtaatcagtaaacgcgggaagtggagtcaggctttttttatggaagagaaaatagacaccaaagtagccttcttctaaccttaacggacctacagtgcaaaaagttatcaagagactgcattatagagcgcacaaaggagaaaaaaagtaatctaagatgctttgttagaaaaatagcgctctcgggatgcatttttgtagaacaaaaaagaagtatagattctttgttggtaaaatagcgctctcgcgttgcatttctgttctgtaaaaatgcagctcagattctttgtttgaaaaattagcgctctcgcgttgcatttttgttttacaaaaatgaagcacagattcttcgttggtaaaatagcgctttcgcgttgcatttctgttctgtaaaaatgcagctcagattctttgtttgaaaaattagcgctctcgcgttgcatttttgttctacaaaatgaagcacagatgcttcgttaacaaagatatgctattgaagtgcaagatggaaacgcagaaaatgaaccggggatgcgacgtgcaagattacctatgcaatagatgcaatagtttctccaggaaccgaaatacatacattgtcttccgtaaagcgctagactatatattattatacaggttcaaatatactatctgtttcagggaaaactcccaggttcggatgttcaaaattcaatgatgggtaacaagagcttttcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaaccgaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggcgccattcgccattcaggctgcgcaactgttgggaagggcgatcggtgcgggcctcttcgctattacgccagctggcgaaggggggatgtgctgcaaggcgattaagttgggtaacgccagggttttcccagtcacgacgttgtaaaacgacggccagtgccaagctttctaactgatctatccaaaactgaaaattacattcttgattaggtttatcacaggcaaatgtaatttgtggtattttgccgttcaaaatctgtagaattttctcattggtcacattacaacctgaaaatactttatctacaatcataccattcttataacatgtccccttaatactaggatcaggcatgaacgcatcacagacaaaatcttcttgacaaacgtcacaattgatccctccccatccgttatcacaatgacaggtgtcattttgtgctcttatgggacgatccttattaccgctttcatccggtgatagaccgccacagaggggcagagagcaatcatcacctgcaaacccttctatacactcacatctaccagtgtacgaattgcattcagaaaactgtttgcattcaaaaataggtagcatacaattaaaacatggcgggcacgtatcattgcccttatcttgtgcagttagacgcgaatttttcgaagaagtaccttcaaagaatggggtctcatcttgttttgcaagtaccactgagcaggataataatagaaatgataatatactatagtagagataacgtcgatgacttcccatactgtaattgcttttagttgtgtatttttagtgtgcaagtttctgtaaatcgattaatttttttttctttcctctttttattaaccttaatttttattttagattcctgacttcaactcaagacgcacagatattataacatctgcacaataggcatttgcaagaattactcgtgagtaaggaaagagtgaggaactatcgcatacctgcatttaaagatgccgatttgggcgcgaatcctttattttggcttcaccctcatactattatcagggccagaaaaaggaagtgtttccctccttcttgaattgatgttaccctcataaagcacgtggcctcttatcgagaaagaaattaccgtcgctcgtgatttgtttgcaaaaagaacaaaactgaaaaaacccagacacgctcgacttcctgtcttcctattgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaacaaatataaaacttaattaaaatgaaggcttctattgctttgactgctattgctgctttggctgctaatgcttctgctgcttgtttttctgaaagattgggttatccatgttgtagaggtaatgaagttttctacactgataatgatggtgattggggtgttgaaaatggtaattggtgtggtattggtggtgcttctgctactacttgttggtcacaagctttaggttacccttgttgtacttctacttctgatgttgcttacgttgatggtgacggtaactggggtgtcgaaaacggtaactggtgcggtataattgcaggtggtaattcttctaacaacaactctggttctactattaatgttggtgatgttactattggtaaccaatacactcatactggtaatccatttgctggtcataaattctttattaacccatactatactgctgaagttgatggtgctattgctcaaatttctaatgcttctttgagagctaaagctgaaaagatgaaagaattttctaacgctatttggttggatactattaagaatatgaacgaatggttggaaaagaatttgaaatatgctttggctgaacaaaatgaaactggtaagactgttttgacagtttttgttgtttatgatttgccaggtagagattgtcatgctttagcttctaatggtgaattgttggctaatgattctgattgggcaagatatcaatctgaatacattgatgttattgaagaaaagttgaaaacttacaagtctcaaccagttgttttggttgttgaaccagattctttggctaatatggttacaaatttggattctactccagcttgtagagattctgaaaaatactatatggatggtcatgcttacttgattaaaaagttgggtgttttgccacatgttgcaatgtatttggatattggtcatgctttttggttgggttgggatgataatagattgaaagctggtaaagtttactctaaggttattcaatctggtgctccaggtaatgttagaggttttgcttctaatgttgctaattatactccatgggaagatccaactttgtctagaggtccagatactgaatggaatccatgtccagatgaaaaaagatacattgaagcaatgtacaaagattttaagtctgctggtattaagtctgtttacttcattgatgatacttctagaaatggtcataagactgatagaactcatccaggtgaatggtgtaatcaaacaggtgttggtattggtgctagaccacaagctaatccaatttctggtatggattacttggatgctttttattgggttaaaccattgggtgaatctgatggttattctgatactactgctgtcagatatgatggttattgtggtcatgctactgctatgaaaccagctcctgaagctggtcaatggtttcaaaaacatttcgaacaaggtttggaaaatgctaatccaccattgttataaggcgcgccgaattcgagagactcgagactgaatcggatcgatcccgggcccgtcgagggatctgcgatagatcaatttttttcttttctctttccccatcctttacgctaaaataatagtttattttattttttgaatattttttatttatatacgtatatatagactattatttatcttttaatgattattaagatttttattaaaaaaaaattcgctcctcttttaatgcctttatgcagtttttttttcccattcgatatttctatgttcgggttcagcgtattttaagtttaataactcgaaaattctgcgttcgttaaagcttgcatgcctgcaggtcgactctagaggatccccgggtaccgagctcgaattaattcgtaatcatggtcat

(19) United States
(12) Patent Application Publication
(10) Pub. No.: US 2013/0230888 A1
(43) Pub. Date: Sep. 5, 2013

(54) Heterologous Expression of Fungal Cellobiohydrolase 2 Genes in Yeast

(71) Applicants: Riaan DEN HAAN, Durbanville (ZA); Emile Van Zyl, Stellenbosch (ZA)

(72) Inventors: Riaan DEN HAAN, Durbanville (ZA); Emile Van Zyl, Stellenbosch (ZA)

(21) Appl. No.: 13/456,804

(22) Filed: Jul. 3, 2012

(30) Foreign Application Priority Data
Oct. 25, 2010 (IB) PCT/IB2010/002904

(51) Int. Cl. C12N 9/24

(52) U.S. Cl. CPC C12N 9/2437
USPC 435/99; 536/23.2; 435/320.1; 435/165; 435/325; 435/254.2; 435/254.21; 435/252.3; 435/252.33; 435/252.35; 435/254.11; 435/419; 435/254.22; 435/254.23; 435/254.8

(57) ABSTRACT

The present invention provides for heterologous expression of polypeptides encoded by wild-type and codon-optimized cbh2 genes from the organisms Cochliobolus heterostrophus, Gibberella zeae, Irpex lacteus, Volvariella volvacea, and Piromyces sp. in host cells, such as the yeast Saccharomyces cerevisiae. The expression in such host cells of the corresponding genes, and variants and combinations thereof, result in improved specific activity of the expressed cellobiohydrolases. Thus, such genes and expression systems are useful for efficient and cost-effective consolidated bioprocessing systems.

### BACKGROUND OF THE INVENTION

Lignocellulosic biomass is widely recognized as a promising source of raw material for production of renewable fuels and chemicals. The primary obstacle impeding the more widespread production of energy from biomass feedstocks is the general absence of low-cost technology for overcoming the recalcitrance of these materials to conversion into useful fuels. Lignocellulosic biomass contains carbohydrate fractions (e.g., cellulose and hemicellulose) that can be converted into ethanol. In order to convert these fractions, the cellulose and hemicellulose must ultimately be converted or hydrolyzed into monosaccharides; it is the hydrolysis that has historically proven to be problematic.

Biologically mediated processes are promising for energy conversion, in particular for the conversion of lignocellulosic biomass into fuels. Biomass processing schemes involving enzymatic or microbial hydrolysis commonly involve four biologically mediated transformations: (1) the production of saccharolytic enzymes (cellulases and hemicellulases); (2) the hydrolysis of carbohydrate components present in pretreated biomass to sugars; (3) the fermentation of hexose sugars (e.g., glucose, mannose, and galactose); and (4) the fermentation of pentose sugars (e.g., xylose and arabinose). These four transformations occur in a single step in a process configuration called consolidated bioprocessing (CBP), which is distinguished from other less highly integrated configurations in that it does not involve a dedicated process step for cellulase and/or hemicellulose production. CBP offers the potential for lower cost and higher efficiency than processes featuring dedicated cellulase production. The benefits result in part from avoided capital costs, substrate and other raw materials, and utilities associated with cellulase production.

Bakers' yeast (Saccharomyces cerevisiae) remains the preferred micro-organism for the production of ethanol (Van Zyl, W. H., et al., Adv. Biochem. Eng. Biotechnol. 108, 205-235 (2007)). Attributes in favor of this microbe are (i) high productivity at close to theoretical yields (0.51 g ethanol produced/g glucose used), (ii) high osmo- and ethanol tolerance, (iii) natural robustness in industrial processes, (iv) being generally regarded as safe (GRAS) due to its long association with wine and bread making and beer brewing. Furthermore, S. cerevisiae exhibits tolerance to inhibitors commonly found in hydrolyzates resulting from biomass pretreatment. The major shortcoming of S. cerevisiae is its inability to utilize complex polysaccharides such as cellulose, or its break-down products, such as cellobiose and cellodextrins. One strategy for developing CBP-enabling microorganisms such as S. cerevisiae is by engineering them to express a heterologous cellulase and/or a hemicelluase system.

Three major types of enzymatic activities are required for native cellulose degradation: The first type is endoglucanases (1,4-β-D-glucan 4-glucanohydrolases; EC 3.2.1.4). Endoglucanases (Eg) cut at random in the cellulose polysaccharide chain of amorphous cellulose, generating oligosaccharides of varying lengths and consequently new chain ends. The second type is β-glucosidases (β-glucoside glucohydrolases; EC 3.2.1.21). β-Glucosidases (Bgl) hydrolyze soluble cellodextrins and cellobiose to glucose units. The third type is exoglucanases. Exogluconases include cellodextrinases (1,4-β-D-glucan glucanohydrolases; EC 3.2.1.74) and cellobiohydrolases (1,4-β-D-glucan cellobiohydrolases; EC 3.2.1.91). Exoglucanases act in a processive manner on the reducing or non-reducing ends of cellulose polysaccharide chains, liberating either glucose (glucanohydrolases) or cellobiose (cellobiohydrolase) as major products. Exoglucanases can also act on microcrystalline cellulose, presumably peeling cellulose chains from the microcrystalline structure. Classically, exoglucanases such as the cellobiohydrolases (Cbh) possess tunnel-like active sites, which can only accept a substrate chain via its terminal regions. These exo-acting Cbh enzymes act by threading the cellulose chain through the tunnel, where successive cellobiose units are removed in a sequential manner. Sequential hydrolysis of a cellulose chain is termed “processivity.”

Structurally, cellulases generally consist of a catalytic domain joined to a cellulose-binding module (CBM) via a linker region that is rich in proline and/or hydroxy-amino acids. In type I exoglucanases, the CBM domain is found at the C-terminal extremity of these enzyme (this short domain forms a hairpin loop structure stabilised by 2 disulphide bridges). In type 2 CBHs, the CBM is found at the N-terminus. In some cases, however, cellulases do not contain a CBM, and only contain a catalytic domain. Examples of such CBM-lacking cellulases include Cbhs from Humicola grisea, Phanerochaete chrysosporium and Aspergillus niger. Grassick et al., Eur. J. Biochem. 271: 4495-4506 (2004).

Cbh2s are classified as family 6 glycosyl hydrolases. Glycosyl hydrolases are a widespread group of enzymes that hydrolyse the glycosidic bond between two or more carbohydrates, or between a carbohydrate and a non-carbohydrate moiety. A classification system for glycosyl hydrolases, based on sequence similarity, has led to the definition of 85 different families (Henrissat, B. et al., Proc. Natl. Acad. Sci. 92:7090-7094 (1995); Davies, G. and Henrissat, B., Structure 3: 853-859 (1995)). Glycoside hydrolase family 6 (GHF6) comprises enzymes with several known activities including endoglucanase (EC:3.2.1.4) and cellobiohydrolase (EC:3.2.1.91).

With the aid of recombinant DNA technology, several of these heterologous cellulases from bacterial and fungal sources have been transferred to S. cerevisiae, enabling the degradation of cellulosic derivatives (Van Rensburg, P., et al., Yeast 14: 67-76 (1998)), or growth on cellobiose (Van Rooyen, R., et al., J. Biotech. 120, 284-295 (2005)); McBride, J. E., et al., Enzyme Microb. Techol. 37, 93-101 (2005)).

Related work was described by Fujita, Y., et al., (Appl. Environ. Microbiol. 70, 1207-1212 (2004)) where cellulases immobilized on the yeast cell surface had significant limitations. First, Fujita et al. were unable to achieve fermentation of amorphous cellulose using yeast expressing only recombinant Bgl1 and EgII. A second limitation of the Fujita et al. approach was that cells had to be pre-grown to high cell density on standard carbon sources before the cells were useful for ethanol production using amorphous cellulose (e.g., Fujita et al. uses high biomass loadings of ˜15 g/L to accomplish ethanol production).

As noted above, ethanol producing yeast such as S. cerevisiae require addition of external cellulases when cultivated on cellulosic substrates, such as pre-treated wood, because this yeast does not produce endogenous cellulases. Expression of fungal cellulases such as T. reesei Cbh1 and Cbh2 in yeast S. cerevisiae has been shown to be functional. Den Haan, R., et al., Enzyme and Microbial Technology 40:1291-1299 (2007). However, current levels of expression and specific activity of cellulases heterologously expressed in yeast are still not sufficient to enable growth and ethanol production by yeast on cellulosic substrates without externally added enzymes. While studies have shown that perhaps certain cellulases, such as T. reesei Cbh1, have some activity when heterologously expressed, there remains a significant need for improvement in the specific activity of heterologously expressed Cbhs in order to attain the goal of achieving a consolidated bioprocessing (CBP) system capable of efficiently and cost-effectively converting cellulosic substrates to ethanol.

Currently, there is no reliable way to predict which cellulases will be efficiently expressed in heterologous organisms. For example, despite the fact that T. reesei Cbh1 and T. emersonii Cbh1 are both endogenously expressed at high levels, heterologous expression of these proteins in yeast yielded disparate results. T. emersonii Cbh1 expression in yeast was significantly greater in yeast than T. reesei Cbh1 under similar conditions. See International Application No. PCT/IB2009/005881, filed May 11, 2009. Efficient expression may depend, for example, on chaperone proteins that differ in the heterologous organisms and in the cellulase's native organism. Furthermore, even cellulases which are expressed at high levels may not be particularly active in a heterologous organism. For example a cellulase may be subject to different post-translational modifications in the heterologous host organism than the in native organism from which the cellulase is derived. Protein folding and secretion can also be a barrier to heterologous cellulase expression.

Therefore, in order to address the limitations of heterologous Cbh expression in consolidated bioprocessing systems, the present invention provides for heterologous expression of wild-type and codon-optimized variants of Cbh2 from the fungal organisms Cochliobolus heterostrophus, Gibberella zeae, Irpex lacteus, Volvariella volvacea, and Piromyces sp. in host cells, such as the yeast Saccharomyces cerevisiae. The expression in such host cells of the corresponding genes, and variants and combinations thereof, result in improved specific activity of the expressed cellobiohydrolases. Thus, such genes and expression systems are useful for efficient and cost-effective consolidated bioprocessing systems.

### BRIEF SUMMARY OF THE INVENTION

The present invention provides for the heterologous expression of Cochliobolus heterostrophus, Gibberella zeae, Irpex lacteus, Volvariella volvacea, and Piromyces sp. cellobiohydrolases, or fragments thereof in host cells, such as the yeast Saccharomyces cerevisiae. The cellobiohydrolase can be a Cbh2, such as Cochliobolus heterostrophus C4 cel7, Gibberella zeae K59 cel6, Irpex lacteus MC-2 cex3, Volvariella volvacea cbhII-1s, and Piromyces sp E2 cel6A.

The Cbh2 expressed in host cells of the present invention is encoded by a wild-type or codon-optimized Cochliobolus heterostrophus, Gibberella zeae, Irpex lacteus, Volvariella volvacea, or Piromyces sp. cbh2 polynucleotide. Codon-optimized polynucleotides can have a codon adaptation index (CAI) of about 0.8 to 1.0, about 0.9 to 1.0, or about 0.95 to 1.0.

Thus, the present invention further provides for an isolated polynucleotide comprising a nucleic acid at least about 70%, 75%, or 80% identical, at least about 90% to about 95% identical, or at least about 96%, 97%, 98%, 99% or 100% identical to a wild-type or codon-optimized Cochliobolus heterostrophus, Gibberella zeae, Irpex lacteus, Volvariella volvacea, or Piromyces sp. cbh2 polynucleotide, or a fragment thereof. In particular aspects, the Cochliobolus heterostrophus, Gibberella zeae, Irpex lacteus, Volvariella volvacea, or Piromyces sp. cbh2 is selected from the group consisting of SEQ ID NOs:1-10, or fragments, variants, or derivatives thereof. Fragments of the Cbh2s include domains such as signal peptides, cellulose binding modules (CBM), and GH family 6 domains.

In further aspects, the present invention encompasses host cells comprising heterologous polypeptides which comprise, or alternatively consist of, an amino acid sequence which is at least 80%, 85%, 90%, 95%, 96%, 97%, 98%, 99% identical to a Cochliobolus heterostrophus, Gibberella zeae, Irpex lacteus, Volvariella volvacea, or Piromyces sp. Cbh2, or domain, fragment, variant, or derivative thereof. In particular embodiments, the Cochliobolus heterostrophus, Gibberella zeae, Irpex lacteus, Volvariella volvacea, or Piromyces sp. Cbh2 is selected from the group consisting of SEQ ID NOs: 11-15.

In further aspects, the present invention encompasses vectors comprising a polynucleotide of the present invention. Such vectors include plasmids for expression in yeast, such as the yeast Saccharomyces cerevisiae. Yeast vectors can be YIp (yeast integrating plasmids), YRp (yeast replicating plasmids), YCp (yeast replicating plasmids with cetromere (CEN) elements incorporated), YEp (yeast episomal plasmids), or YLp (yeast linear plasmids). In certain aspects, these plasmids contain two types of selectable genes: plasmid-encoded drug-resistance genes and cloned yeast genes, where the drug resistant gene is typically used for selection in bacterial cells and the cloned yeast gene is used for selection in yeast. Drug-resistance genes include, for example, ampicillin, kanamycin, tetracycline, and neomycin. Cloned yeast genes include, for example, HIS3, LEU2, LYS2, TRP1, URA3 and TRP1. In some embodiments of the present invention, the vector is a plasmid. For example, the plasmid can be a yeast episomal plasmid or a yeast integrating plasmid.

In particular embodiments, the vector of the present invention is selected from the group consisting of pRDH150, pRDH151, pRDH152, pRDH153, and pRDH154.

In certain additional embodiments, the vector comprises a first polynucleotide encoding for a Cochliobolus heterostrophus, Gibberella zeae, Irpex lacteus, Volvariella volvacea, or Piromyces sp. Cbh2 and a second polynucleotide encoding for a CBM domain, for example, the CBM domain of T. reesei Cbh2 or T. reesei Cbh2.

In other embodiments of the present invention the first and second polynucleotides are contained in a single linear DNA construct. The first and second polynucleotides in the linear DNA construct can be in the same or different expression cassette.

In further embodiments, the first and second polynucleotides are in the same orientation, or the second polynucleotide is in the reverse orientation of the first polynucleotide. In additional embodiments, the first polynucleotide is either N-terminal or C-terminal to the second polynucleotide.

In certain other embodiments, the first polynucleotide and/or the second polynucleotide are encoded by codon-optimized polynucleotides, for example, polynucleotides codon-optimized for S. cerevisiae.

The present invention further provides for a host cell comprising a polynucleotide or a vector of the present invention from which a Cochliobolus heterostrophus, Gibberella zeae, Irpex lacteus, Volvariella volvacea, or Piromyces sp. cellobiohydrolase, e.g. a Cbh2, is heterologously expressed. In certain aspects, the host cell is a yeast such as Saccharomyces cerevisiae. In additional embodiments, the host cell further comprises one or more heterologously expressed endoglucanase polypeptides and/or one or more heterologously expressed β-glucosidase polypeptides and/or one or more heterologously expressed cellobiohydrolase polypeptides. In particular aspects, the endoglucanase polypeptide is a C. formosanus Eg1, the β-glucosidase polypeptide is S. fibuligera Bgl1, and/or the cellobiohdyrolase I is T. emersonii cellobiohdyrolase I.

The present invention further provides for a co-culture of host cells wherein a first cell comprising a first heterologous cellulase selected from the group consisting of a Cochliobolus heterostrophus, Gibberella zeae, Irpex lacteus, Volvariella volvacea, or Piromyces sp. cellobiohydrolase, e.g. a Cbh2, is cultured with a host cell expressing a second heterologous cellulase. The second heterologous cellulase can be, for example, an endoglucanase, a β-glucosidase, and/or a cellobiohydrolase. In particular aspects, the endoglucanase polypeptide is a C. formosanus Eg1, the β-glucosidase polypeptide is S. fibuligera Bgl1, and/or the cellobiohdyrolase I is T. emersonii cellobiohdyrolase I.

The present invention further provides for a method for hydrolyzing a cellulosic substrate, comprising contacting said cellulosic substrate with a host cell according to the present invention. In certain aspects, the cellulosic substrate is of a lignocellulosic biomass. Heterologous expression of Cochliobolus heterostrophus, Gibberella zeae, Irpex lacteus, Volvariella volvacea, or Piromyces sp. Cbh2 in host cells will augment cellulose hydrolysis and facilitate ethanol production by those host cells on cellulosic substrates.

### BRIEF DESCRIPTION OF THE DRAWINGS/FIGURES

FIG. 1 depicts a plasmid map of pMU784. The pMU784 plasmid includes the Clcbh2b gene under the control of the S. cerevisiae PGK1 promoter/terminator. The gene encoding Clcbh2b was excised by means of digestions with the restriction endonucleases Pad and AscI and replaced with the alternate cellobiohydrolase 2 genes listed in Table 8. The plasmid also includes origin of replication (ori) and bla (ampicillin resistance) sequences for replication and maintenance of the plasmid in E. coli. In addition, an S. ceriviseae URA3 gene, as well as a 2-micron origin of replication are in the plasmid for selecting and replication of the plasmid in yeast.

FIG. 2 depicts an SDS-Page analysis of the supernatants of Cbh2 producing strains. A strain containing a plasmid with no foreign gene was used as reference strain (REF), and the strain expressing the unmodified T. emersonii cbh1 (pRDH105) was included as a positive control. The strain containing the plasmid pMU784 expressing C. lucknowense cbh2b was also included as a positive control. Other vector names (RDH150-RDH154) refer to the plasmids expressing the genes as listed in Table 8.

FIG. 3 depicts a bar graph showing activity of strains expressing Cbh2s on Avicel. The % Avicel hydrolysis (starting with a 1% Avicel concentration) was measured for the reference strain (REF) and strains containing a plasmid encoding a heterologous Cbh2 at 24 and 48 hour time points.

FIG. 4 depicts a bar graph showing protein levels measured using the Bradford method (BioRad). The concentrations of the total extracellular protein and the secreted Cbh2 proteins were determined for the reference strain (REF) and strains containing a plasmid encoding a heterologous Cbh2. The amount of secreted Cbh2 protein measured in the reference strain was deducted from each of the secreted Cbh2 measurements.

FIG. 5 depicts a bar graph showing the specific activity of heterologously expressed Cbh2s. The % Avicel hydrolysis per microgram of Cbh2 was measured for the reference strain (REF) and strains containing a plasmid encoding a heterologous Cbh2.

### DETAILED DESCRIPTION OF THE INVENTION

The present invention relates to, inter alia, the heterologous expression of cbh2 genes from Cochliobolus heterostrophus, Gibberella zeae, Irpex lacteus, Volvariella volvacea, and Piromyces sp. in host cells, including yeast, e.g., Saccharomyces cerevisiae. The present invention provides important tools to enable growth of yeast on cellulosic substrates for production of products such as ethanol.

### DEFINITIONS

A “vector,” e.g., a “plasmid” or “YAC” (yeast artificial chromosome) refers to an extrachromosomal element often carrying one or more genes that are not part of the central metabolism of the cell. They can be in the form of a circular double-stranded DNA molecule. Such elements can be autonomously replicating sequences, genome integrating sequences, or phage sequences. Such elements can be linear, circular, or supercoiled and can be single- or double-stranded. They can also be DNA or RNA, derived from any source. They can include a number of nucleotide sequences which have been joined or recombined into a unique construction which is capable of introducing a promoter fragment and DNA sequence for a selected gene product along with appropriate 3′ untranslated sequence into a cell. The plasmids or vectors of the present invention can be stable and self-replicating. The plasmids or vectors of the present invention can also be suicide vectors, or vectors that cannot replicate in the host cell. Such vectors are useful for forcing insertion of the nucleotide sequence into the host chromosome.

An “expression vector” is a vector that is capable of directing the expression of at least one polypeptide encoded by a polynucleotide sequence of the vector.

The term “heterologous” as used herein refers to an element of a vector, plasmid or host cell that is derived from a source other than the endogenous source. Thus, for example, a heterologous sequence could be a sequence that is derived from a different gene or plasmid from the same host, from a different strain of host cell, or from an organism of a different taxonomic group (e.g., different kingdom, phylum, class, order, family genus, or species, or any subgroup within one of these classifications). The term “heterologous” is also used synonymously herein with the term “exogenous.”

The term “domain” as used herein refers to a part of a molecule or structure that shares common physical or chemical features, for example hydrophobic, polar, globular, helical domains or properties, e.g., a DNA binding domain or an ATP binding domain. Domains can be identified by their homology to conserved structural or functional motifs. Examples of cellobiohydrolase (CBH) domains include the catalytic domain (CD) and the carbohydrate binding module (CBM).

A “nucleic acid,” “polynucleotide,” or “nucleic acid molecule” is a polymeric compound comprised of covalently linked subunits called nucleotides. Nucleic acid includes polyribonucleic acid (RNA) and polydeoxyribonucleic acid (DNA), both of which can be single-stranded or double-stranded. DNA includes cDNA, genomic DNA, synthetic DNA, and semi-synthetic DNA.

An “isolated nucleic acid molecule” or “isolated nucleic acid fragment” refers to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogs thereof, such as phosphorothioates and thioesters, in either single stranded faun, or a double-stranded helix. Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alfa, in linear or circular DNA molecules (e.g., restriction fragments), plasmids, and chromosomes. In discussing the structure of particular double-stranded DNA molecules, sequences are generally described herein according to the normal convention of giving only the sequence in the 5′ to 3′ direction along the non-transcribed strand of DNA (i.e., the strand having a sequence homologous to the mRNA).

A “gene” refers to an assembly of nucleotides that encode a polypeptide, and includes cDNA and genomic DNA nucleic acids. “Gene” also refers to a nucleic acid fragment that expresses a specific protein, including intervening sequences (introns) between individual coding segments (exons), as well as regulatory sequences preceding (5° non-coding sequences) and following (3′ non-coding sequences) the coding sequence. “Native gene” refers to a gene as found in nature with its own regulatory sequences.

A nucleic acid molecule is “hybridizable” to another nucleic acid molecule, such as a cDNA, genomic DNA, or RNA, when a single stranded form of the nucleic acid molecule can anneal to the other nucleic acid molecule under the appropriate conditions of temperature and solution ionic strength. Hybridization and washing conditions are well known and exemplified, e.g., in Sambrook, J., Fritsch, E. F. and Maniatis, T. MOLECULAR CLONING: A LABORATORY MANUAL, Second Edition, Cold Spring Harbor Laboratory Press, Cold Spring Harbor (1989), particularly Chapter 11 and Table 11.1 therein (hereinafter “Maniatis”, entirely incorporated herein by reference). The conditions of temperature and ionic strength determine the “stringency” of the hybridization. Stringency conditions can be adjusted to screen for moderately similar fragments, such as homologous sequences from distantly related organisms, to highly similar fragments, such as genes that duplicate functional enzymes from closely related organisms. Post-hybridization washes determine stringency conditions. One set of conditions uses a series of washes starting with 6×SSC, 0.5% SDS at room temperature for 15 min, then repeated with 2×SSC, 0.5% SDS at 45° C. for 30 min, and then repeated twice with 0.2×SSC, 0.5% SDS at 50° C. for 30 min. For more stringent conditions, washes are performed at higher temperatures in which the washes are identical to those above except for the temperature of the final two 30 min washes in 0.2×SSC, 0.5% SDS are increased to 60° C. Another set of highly stringent conditions uses two final washes in 0.1×SSC, 0.1% SDS at 65° C. An additional set of highly stringent conditions are defined by hybridization at 0.1×SSC, 0.1% SDS, 65° C. and washed with 2×SSC, 0.1% SDS followed by 0.1×SSC, 0.1% SDS.

Hybridization requires that the two nucleic acids contain complementary sequences, although depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the greater the value of Tm for hybrids of nucleic acids having those sequences. The relative stability (corresponding to higher Tm) of nucleic acid hybridizations decreases in the following order: RNA:RNA, DNA:RNA, DNA:DNA. For hybrids of greater than 100 nucleotides in length, equations for calculating Tm have been derived (see, e.g., Maniatis at 9.50-9.51). For hybridizations with shorter nucleic acids, i.e., oligonucleotides, the position of mismatches becomes more important, and the length of the oligonucleotide determines its specificity (see, e.g., Maniatis at 11.7-11.8). In one embodiment the length for a hybridizable nucleic acid is at least about 10 nucleotides. A minimum length for a hybridizable nucleic acid can also be at least about 15 nucleotides, at least about 20 nucleotides, or at least 30 nucleotides. Furthermore, the skilled artisan will recognize that the temperature and wash solution salt concentration can be adjusted as necessary according to factors such as length of the probe.

The term “percent identity”, as known in the art, is a relationship between two or more polypeptide sequences or two or more polynucleotide sequences, as determined by comparing the sequences. In the art, “identity” also means the degree of sequence relatedness between polypeptide or polynucleotide sequences, as determined by the match between strings of such sequences.

By a nucleic acid having a nucleotide sequence at least, for example, 95% “identical” to a reference nucleotide sequence of the present invention, it is intended that the nucleotide sequence of the nucleic acid is identical to the reference sequence except that the nucleotide sequence can include up to five point mutations per each 100 nucleotides of the reference nucleotide sequence encoding the particular polypeptide. In other words, to obtain a nucleic acid having a nucleotide sequence at least 95% identical to a reference nucleotide sequence, up to 5% of the nucleotides in the reference sequence can be deleted or substituted with another nucleotide, or a number of nucleotides up to 5% of the total nucleotides in the reference sequence can be inserted into the reference sequence.

As a practical matter, whether any particular nucleic acid molecule or polypeptide is at least 80%, 85%, 90%, 95%, 96%, 97%, 98% or 99% identical to a nucleotide sequence or polypeptide of the present invention can be determined conventionally using known computer programs. A method for determining the best overall match between a query sequence (a sequence of the present invention) and a subject sequence, also referred to as a global sequence alignment, can be determined using the FASTDB computer program based on the algorithm of Brutlag et al., Comp. App. Biosci. (1990) 6:237-245. In a sequence alignment the query and subject sequences are both DNA sequences. An RNA sequence can be compared by converting U's to T's. The result of said global sequence alignment is in percent identity. Preferred parameters used in a FASTDB alignment of DNA sequences to calculate percent identity are: Matrix=Unitary, k-tuple=4, Mismatch Penalty=1, Joining Penalty=30, Randomization Group Length=0, Cutoff Score=1, Gap Penalty=5, Gap Size Penalty 0.05, Window Size=500 or the length of the subject nucleotide sequence, whichever is shorter.

If the subject sequence is shorter than the query sequence because of 5′ or 3′ deletions, not because of internal deletions, a manual correction must be made to the results. This is because the FASTDB program does not account for 5′ and 3′ truncations of the subject sequence when calculating percent identity. For subject sequences truncated at the 5′ or 3′ ends, relative to the query sequence, the percent identity is corrected by calculating the number of bases of the query sequence that are 5′ and 3′ of the subject sequence, which are not matched/aligned, as a percent of the total bases of the query sequence. Whether a nucleotide is matched/aligned is determined by results of the FASTDB sequence alignment. This percentage is then subtracted from the percent identity, calculated by the above FASTDB program using the specified parameters, to arrive at a final percent identity score. This corrected score is what is used for the purposes of the present invention. Only bases outside the 5′ and 3′ bases of the subject sequence, as displayed by the FASTDB alignment, which are not matched/aligned with the query sequence, are calculated for the purposes of manually adjusting the percent identity score.

For example, a 90 base subject sequence is aligned to a 100 base query sequence to determine percent identity. The deletions occur at the 5′ end of the subject sequence and therefore, the FASTDB alignment does not show a matched/alignment of the first 10 bases at 5′ end. The 10 unpaired bases represent 10% of the sequence (number of bases at the 5′ and 3′ ends not matched/total number of bases in the query sequence) so 10% is subtracted from the percent identity score calculated by the FASTDB program. If the remaining 90 bases were perfectly matched the final percent identity would be 90%. In another example, a 90 base subject sequence is compared with a 100 base query sequence. This time the deletions are internal deletions so that there are no bases on the 5′ or 3′ of the subject sequence which are not matched/aligned with the query. In this case the percent identity calculated by FASTDB is not manually corrected. Once again, only bases 5′ and 3′ of the subject sequence which are not matched/aligned with the query sequence are manually corrected for. No other manual corrections are to be made for the purposes of the present invention.

As known in the art, “similarity” between two polypeptides is determined by comparing the amino acid sequence and conserved amino acid substitutes thereto of the polypeptide to the sequence of a second polypeptide.

Suitable nucleic acid sequences or fragments thereof (isolated polynucleotides of the present invention) encode polypeptides that are at least about 70% to 75% identical to the amino acid sequences reported herein, at least about 80%, 85%, or 90% identical to the amino acid sequences reported herein, or at least about 95%, 96%, 97%, 98%, 99%, or 100% identical to the amino acid sequences reported herein. Suitable nucleic acid fragments are at least about 70%, 75%, or 80% identical to the nucleic acid sequences reported herein, at least about 80%, 85%, or 90% identical to the nucleic acid sequences reported herein, or at least about 95%, 96%, 97%, 98%, 99%, or 100% identical to the nucleic acid sequences reported herein. Suitable nucleic acid fragments not only have the above identities/similarities but typically encode a polypeptide having at least 50 amino acids, at least 100 amino acids, at least 150 amino acids, at least 200 amino acids, at least 250 amino acids, at least 300 amino acids, or at least 350 amino acids.

The term “probe” refers to a single-stranded nucleic acid molecule that can base pair with a complementary single stranded target nucleic acid to form a double-stranded molecule.

The term “complementary” is used to describe the relationship between nucleotide bases that are capable to hybridizing to one another. For example, with respect to DNA, adenosine is complementary to thymine and cytosine is complementary to guanine. Accordingly, the instant invention also includes isolated nucleic acid fragments that are complementary to the complete sequences as reported in the accompanying Sequence Listing as well as those substantially similar nucleic acid sequences.

As used herein, the term “oligonucleotide” refers to a nucleic acid, generally of about 18 nucleotides, that is hybridizable to a genomic DNA molecule, a cDNA molecule, or an mRNA molecule. Oligonucleotides can be labeled, e.g., with 32P-nucleotides or nucleotides to which a label, such as biotin, has been covalently conjugated. An oligonucleotide can be used as a probe to detect the presence of a nucleic acid according to the invention. Similarly, oligonucleotides (one or both of which can be labeled) can be used as PCR primers, either for cloning full length or a fragment of a nucleic acid of the invention, or to detect the presence of nucleic acids according to the invention. Generally, oligonucleotides are prepared synthetically, for example, on a nucleic acid synthesizer. Accordingly, oligonucleotides can be prepared with non-naturally occurring phosphoester analog bonds, such as thioester bonds, etc.

A DNA or RNA “coding region” is a DNA or RNA molecule which is transcribed and/or translated into a polypeptide in a cell in vitro or in vivo when placed under the control of appropriate regulatory sequences. “Suitable regulatory regions” refer to nucleic acid regions located upstream (5′ non-coding sequences), within, or downstream (3′ non-coding sequences) of a coding region, and which influence the transcription, RNA processing or stability, or translation of the associated coding region. Regulatory regions can include promoters, translation leader sequences, RNA processing site, effector binding site and stem-loop structure. The boundaries of the coding region are determined by a start codon at the 5′ (amino) terminus and a translation stop codon at the 3′ (carboxyl) terminus. A coding region can include, but is not limited to, prokaryotic regions, cDNA from mRNA, genomic DNA molecules, synthetic DNA molecules, or RNA molecules. If the coding region is intended for expression in a eukaryotic cell, a polyadenylation signal and transcription termination sequence will usually be located 3′ to the coding region.

“Open reading frame” is abbreviated ORF and means a length of nucleic acid, either DNA, cDNA or RNA, that comprises a translation start signal or initiation codon, such as an ATG or AUG, and a termination codon and can be potentially translated into a polypeptide sequence.

“Promoter” refers to a DNA fragment capable of controlling the expression of a coding sequence or functional RNA. In general, a coding region is located 3′ to a promoter. Promoters can be derived in their entirety from a native gene, or be composed of different elements derived from different promoters found in nature, or even comprise synthetic DNA segments. It is understood by those skilled in the art that different promoters can direct the expression of a gene in different tissues or cell types, or at different stages of development, or in response to different environmental or physiological conditions. Promoters which cause a gene to be expressed in most cell types at most times are commonly referred to as “constitutive promoters”. It is further recognized that since in most cases the exact boundaries of regulatory sequences have not been completely defined, DNA fragments of different lengths can have identical promoter activity. A promoter is generally bounded at its 3′ terminus by the transcription initiation site and extends upstream (5′ direction) to include the minimum number of bases or elements necessary to initiate transcription at levels detectable above background. Within the promoter will be found a transcription initiation site (conveniently defined for example, by mapping with nuclease S1), as well as protein binding domains (consensus sequences) responsible for the binding of RNA polymerase.

A coding region is “under the control” of transcriptional and translational control elements in a cell when RNA polymerase transcribes the coding region into mRNA, which is then trans-RNA spliced (if the coding region contains introns) and translated into the protein encoded by the coding region.

“Transcriptional and translational control regions” are DNA regulatory regions, such as promoters, enhancers, terminators, and the like, that provide for the expression of a coding region in a host cell. In eukaryotic cells, polyadenylation signals are control regions.

The term “operably associated” refers to the association of nucleic acid sequences on a single nucleic acid fragment so that the function of one is affected by the other. For example, a promoter is operably associated with a coding region when it is capable of affecting the expression of that coding region (i.e., that the coding region is under the transcriptional control of the promoter). Coding regions can be operably associated to regulatory regions in sense or antisense orientation.

The term “expression,” as used herein, refers to the transcription and stable accumulation of sense (mRNA) or antisense RNA derived from the nucleic acid fragment of the invention. Expression can also refer to translation of mRNA into a polypeptide.

### Polynucleotides of the Invention

The present invention provides for the use of cbh2 polynucleotide sequences from Cochliobolus heterostrophus, Gibberella zeae, Irpex lacteus, Volvariella volvacea, and Piromyces sp.

The Cochliobolus heterostrophus C4 cel7, Gibberella zeae K59 cel6, Irpex lacteus MC-2 cex3, Volvariella volvacea cbhII-I, and Piromyces sp. E2 cel6A nucleic acid sequences are available in GenBank, and are shown in Table 1 below.

TABLE 1
Polynucleotide sequences encoding Cbh2s.

Cochliobolus heterostrophus C4 ce17 (GenBank accession: AY116307)
cttcttggttctcaaagatgctctccaacgtctttcttaccgctgccctcgcagccggcctggctcaggccctgccccaggccacgcctaccc
caaccgctgcgccctctggcaaccccttcgcgggcaagaacttctacgccaacccatactactcgtctgaagtccacaccctggccatgcc
ctcgcttccagcctcgctgaagcctgctgctaccgccgtggccaaggtcggatcattcgtgtggatggacaccatggccaaggttcctctcat
ggacacctacctcgcagacatcaaggccaagaacgctgctggcgcaaacctcatgggtacttttgtcgtctacgaccttcccgaccgtgact
gcgccgctctggcctccaacggtgaactcaagattgacgagggtggtgtcgagaagtacaagacccagtacattgacaagattgccgccat
catcaagaagtaccccgacgtcaagatcaaccttgccattgagcccgattcccttgccaacatggtcaccaacatgggtgtgcagaagtgct
cgcgcgccgccccatactacaaggagctcactgcctacgccctcaagacgctcaacttcaacaacgtcgacatgtacatggacggtggcc
acgccggttggctcggctgggacgccaacattggccctaccgcaaagcttttcgcagaggtctacaaggctgctggctctccccgtggcgt
ccgtggtatcgtcaccaacgtcagcaactacaacgctctccgcgtctcctcctgcccatccatcacccaaggaaacaagaactgcgacgag
gagcgctacatcaacgccttggctcctcttctcaagaacgagggtttccctgctcacttcatcgtcgaccagggccgctccggaaaggtgcct
actaaccagcaggagtggggtgactggtgcaacgtctcaggtgctggattcggtacccgtcccaccaccaacactggcaatgccctcattg
atgccatcgtctgggtcaagcccggtggcgagtctgacggtacctctgacaccagcgctgcccgctacgatgcccactgcggcaggaaca
gcgctttcaagcccgctcctgaggctggaacctggttccaggcttacttcgagatgcttctcaagaacgctaaccctgctcttgcttaagtgtct
ggttcttttgaataagcttgggtagattgttagaagggaaaattagtctgcgagtggtctttcaccgcagattctggtggattgtaaatatggct
ttggaactagaataggcaacgtttgatgttgcagttcgtgtaaatattataccttttggagctaaaaaaaaaaaaaaaaaa
(SEQ ID NO: 1)
Gibberella zeae K59 cel6 (GenBank Accession AY302753)
atgacggcctacaagcttttcctggctgctgcttttgcagccactgctctcgcagctcctgttgaagagcgtcagtcttgcagcaacggagtct
ggtgagtgtttgcagccatctttttaaagaattaattactcacatacccataggtctcaatgtggtggtcagaactggagcggtactccttgctg
caccagtggaaacaagtgtgtcaaggtcaacgacttctactcccaatgccagcctggatccgcagacccttctcccacgagcaccattgtcag
tgccacaaccaccaaggctactaccactggtagtggaggctctgtcacctcgcctcctcctgttgccaccaacaatcccttctctggcgttgat
ctgtgggccaacaactactaccgctccgaggtcagcactctcgctatccccaagctgagcggtgccatggccaccgctgctgccaaggtcg
ccgatgttccttctttccagtggatgtgagttacgagtccctttggatatatacctctttactaaccacgatagggacacttatgaccacatctc
cttcatggaggactctcttgccgatatccgcaaggccaacaaggctggtggcaactacgctggtcagttcgtcgtctacgatcttcccgaccgtg
actgtgctgctgctgcctccaacggagagtactcccttgacaaggatggcaagaacaagtacaaggcctacattgcagatcaagggatcctt
caggactactctgacacccgcatcattctcgttatcggttagtccacctgattgactccgacttagttcctactaacagccatttagagcctgat
tctcttgctaacatggtcaccaacatgaacgtccccaagtgcgccaacgctgctagcgcttacaaggagctcaccattcacgccctcaaggag
ctcaaccttcccaacgtctccatgtacatcgatgcaggtcacggtggctggctgggatggcccgccaaccttcctcctgccgcccagctcta
cggtcagctctacaaggatgccggcaagccatctcgcctccgaggtctcgtcaccaacgtctccaactacaacgcctggaagctgtcctcc
aagcccgactacactgagagcaaccccaactacgacgagcagaagtacatccacgctctatctcctcttctggagcaggagggctggccc
ggtgccaagttcatcgtcgaccagggccgatctggtaagcagcccactggccagaaggcttggggtgactggtgcaacgctcccggaact
ggattcggtctccgaccctctgccaacactggcgatccctcgtcgacgctttcgtctgggtcaagcctggtggtgagtctgatggtacctct
gatacctctgctgctcgctacgactaccactgcggtattgacggcgctgtcaagtaagttttataatacaaatcctcaagttaaccctcatacta
accccgataactaggcccgctcctgaggctggaacctggttccaggcttactttgagcagcttctcaagaacgccaacccctctttcctgtaa
(SEQ ID NO: 2)
Irpex lacteus MC-2 cex3 (GenBank Accession AB370872)
ccgcaccccagcatagcaacagctttttcgtcggcaagatattaagcacggtcatggagttttcaacgacttaaccgagcttgtaccgaagtg
gacggcagttcgctgaacgttcgggtgtgctttttacaacccgtcgttgaaaataatgtgtaggtatggccgtagcctcatgaccccactcata
acgtccgtcgttcagcaactgaccctcccccgacgtctatccgctaacaatgctcgggtctacgccggaattatggtattcttccactggtggg
cctgaacgatgcaaaacggtgcttctgatgagcccacctctgtattatttccggtatataagaagtggtatcgtcggctagggttctacaggatc
cacatcccactgagacgaatccactgcaagtgcaatgaagtccgctgctttcctcgctgctctcgccgccatcctcccagcctatgtcgctgg
ccaagcccagacttgggcacagtgcggtggtatcggcttcagtacgttactacctttctccttctactggtctgttacttactgaacttgcctat
catagctggtcctaccacttgcgttgccggctccgtctgcacgaagcagaatgattactactctcagtgcatgtaagtacgaatccacccttttg
caagaactactgacttatgatggggtatagtcctggatctgctactactcccacatctgcacctacatctgcacccacctcccagccttcgcagc
catcttccacctcctctgctccttccggtccttcctctacccccacgccctctgccaacaacccatggactggctaccaggtatgcgggcgatcc
attgtaactctaaaaatctctttctgacctgacctgggcatagatctacttgagcccttactacgctaacgaggttgctgccgccgccaaggcaa
tcacggaccccaccctcgccgccaaggctgccagcgttgctaacatcccgaacttcacttggttgggtgagtgtgacattgacaagagaag
gaaacgacttcctaattacccgcatagactccgtctccaagatcgctgatcttaagacatacctcgctgacgcaagtgcactgggcaagtcca
gcggtcagaagcaactcctccagattgtcgtatacgatcttcccgaccgtgattgcgctgctaaggcctccaatggagagttcagcattgctg
acaacggcctggccaactaccagaactacatcgaccagatcgttgctgctgtcaagcgtaagtctcgacgaggcagttcacttcgctttgcat
actgagcctgttcgccacagaattccctgacgttcgggtcgtggctgtcattgagcccgactctcttgccaacttggtcaccaacttgaacgtg
cagaagtgcgctaacgccaagagcacctacctcactgccgtcaactacgctttgaagcagctctcctcagttggcgtgtaccagtacatgga
cgcaggtcacgccggatggctcggttggcccgccaacttgacccccgccgctcagctgttcgctcaagtttactctgatgccggaaagtcg
ccattcatcaagggtcttgctaccagtacgttttcatttcgttttgttcgatcactcaagactgacccgcttgaatcgcaaagacgtcgccaact
acaacgccttgagcgcggcctcacccgatcccatcacccagggtgaccccaactacgatgaaatccactacatcaacgtaagcccgtttaac
cgtacaatgcgatgtgtactaatcaaaccaaatcccgcaggctctcgctccggctctccagtccgctggcttccctgctaccttcatcgtcgat
caaggccgttccggtcagcagaaccaccgacaacagtggggtgactggtgcaacatcaagggtgctgggttcggtacccgcccgaccac
caacactggttcttcgctcatcgactccatcgtttgggtgaagcccggaggtgaatccgacggtacctcgaactcgtcttcgccccgtttcgac
tccacttgctctttggtaagttcggccttctgttcgtcaaactgagtgtgatgctaactcatcgtgcttgcagtcggatgctactcagcccgctc
ctgaggccggtacatggttccaggcttacttcgagactctcgtctccaaggccaacccaccgctctaagcgtatcgtacctgctttcaaaatgtg
gctgaacggcatagaacagctgctcttggggttctcttcacttgatcgcgatttttatatacctgtattttatgtagcataaaaagtaaaacagc
cgcagaaatgcattcgcttttcacttgtaccgcgtcttgttcttgtgccaaatgctctcgcgtcctaccgagttcatctttcgatatcagtgagc
ggccagcatcgaaacgaccactgcgttagtttgtctggcgacatctgcatgcaagcta
(SEQ ID NO: 3)
Volvariella volvacea cbhII-I (GenBank Accession AY559104)
tgattgcaagccacatatcccagagatgtccaggttttctgctcttactgctctccttttatctttgccactactggctattgctcagtccccgt
tgtatgggcaatgtggtggcaacggctggactggcccaaagacctgtgtatcaggtgcaacttgtacagtgatcaatgactggtattggcaatgc
ctgccaggaaatggcccaacttcttcttcaccaacttccacacctaccaccaccacaactacagggggacctcaaccaaccgtaccagcagca
gggaatccttatactggatacgagatttacttgagtccttattacgctgctgaggctcaagctgcggctgcccaaatttctgatgccacgcaga
aggccaaagccctgaaggtcgcacaaatccccacattcacctggtttgatgttattgcaaagacctccacactcggtgattatttggccgaag
cgagcgcacttgggaaatcctctggaaagaaatacctcgttcaaatcgttgtatatgacttgccagatcgggattgcgctgctctggcttcgaa
tggagagtttagcatcgcaaacaatgggctcaacaactacaagggctacatcgatcaattggttgctcagatcaagaaataccctgatgtccg
agtcgtggctgttattgaacccgactccttggccaatctcgttaccaatctcaatgttagcaagtgtgccaatgcacaaacagcctacaaggct
ggtgtcacgtacgctctccagcagctcaactctgttggcgtctatatgtacctcgatgctggacatgcgggttggctcggatggcctgccaact
tgaatcccgctgcgcaactgttctctcaattgtacagagatgctggaagtccccaatatgtccgtggcctagctaccaatgttgccaactacaa
cgcactctctgccagcagccccgacccagtcacacaaggcaatcccaactatgacgaacttcattacatcaacgcactcgcgccagctctc
caatccggtggcttccctgcccacttcattgtcgaccaaggccgatcaggagttcagaacatcagacaacaatggggcgactggtgcaacg
tcaagggtgcaggctttggccagcgtccaactcttagcacaggttcatcccttatcgacgccattgtctggattaaacccggaggcgaatgcg
acggtacaaccaacacatcgtcacctcgctatgattctcactgtggtctttctgatgctacacccaatgccccagaagctggccaatggttcca
ggcttacttcgagaccttagtccgtaacgccagcccacctctttgagtgtgcagtgtagataccagatatacaaggccccgagtgtgatacaa
cagaataaataatccctttttgctcctctcaaaaaaaaaaaaaaaaaaaaaa
(SEQ ID NO: 4)
Piromyces sp. E2 cel6A (GenBank Accession AY082395)
aaatcttaattataattaataatatcatttttcatttattatatttatactttgtttcatgaaataataataaacaacattttcccaatagttt
taaaatcattttttacttttctcaaatttatcgaacaattaaaaactataaaaggagcaatttttcattttaattattttcttcattaattaaa
aaattattttctctggaagaaaataaatataatagaaaaaaataaaaagaaaaggaaattacaaaaaaacaaaattaaataatatatattgatt
tatatattaattaaaaataatatatttttaaatttattatcaacaaaaaaaaaaaatttttaatcaaaaaatgaaggcttctattgctttaact
gctattgccgctcttgctgctaacgcttctgctgcttgtttctctgaaagacttggttatccatgttgcagaggtaatgaagttttttacaccg
ataatgatggtgattggggtgttgaaaatggtaactggtgtggtattggtggtgcttctgctactacctgctggtctcaagctttaggttaccc
atgttgtacttctacttccgatgttgcctatgttgatggtgatggcaattggggtgttgaaaatggtaattggtgtggtattattgctggtggt
aattcaagcaacaacaacagtggtagtaccattaatgttggtgatgttaccattggtaatcaatacactcacactggtaatccattcgctggtc
acaaattcttcattaatccatactacactgctgaagtcgatggtgccatcgctcaaatttctaacgcttctcttagagctaaggctgaaaaaat
gaaagaattctctaatgctatctggttagatactattaagaatatgaatgaatggttagaaaagaatcttaaatacgctcttgctgaacaaaat
gaaactggtaagaccgttttaaccgttttcgttgtttacgatttaccaggtcgtgattgtcatgctcttgcttccaatggtgaacttcttgcca
acgacagtgattgggctcgttaccaatcggaatacattgatgtcattgaagaaaaattaaagacttacaagagtcaaccagttgttcttgttgt
tgaaccagattctcttgctaacatggttactaatcttgattctactccagcttgtcgtgattctgaaaagtattacatggatggtcatgcttac
ttaattaaaaagcttggtgttcttccacatgttgctatgtaccttgatattggtcatgctttctggttaggatgggatgataaccgtttaaagg
ctggtaaggtttactccaaggttattcaatctggtgctccaggtaatgttcgtggtttcgcttctaacgttgctaactacactccatgggaaga
tccaactctttctcgtggtccagacactgaatggaatccatgtccagatgaaaagagatacattgaagccatgtacaaggacttcaagtctgct
ggtattaaatccgtttacttcattgatgatacttctcgtaatggtcacaaaaccgaccgtactcatccaggagaatggtgtaaccaaaccggag
ttggtattggtgctcgtccacaagccaatccaatctctggtatggactaccttgatgctttctactgggttaaaccactcggtgaatccgatgg
ttactccgatactacagccgttcgttatgatggttattgtggtcatgctactgccatgaaaccagcaccagaagccggtcaatggttccaaaag
cactttgaacaaggtcttgaaaatgctaatccaccactctaatcatattaacattaaataatatacattatatacatatagaaagaaacatgaa
tattantattaacataatcatacttnttaaataaattatt
(SEQ ID NO: 5)

The present invention also provides for the use of an isolated polynucleotide comprising a nucleic acid at least about 70%, 75%, or 80% identical, at least about 90% to about 95% identical, or at least about 96%, 97%, 98%, 99% or 100% identical to any of SEQ ID NOs:1-5, or fragments, variants, or derivatives thereof.

In certain aspects, the present invention relates to a polynucleotide comprising a nucleic acid encoding a functional and/or structural domain of a Cochliobolus heterostrophus, Gibberella zeae, Irpex lacteus, Volvariella volvacea, or Piromyces sp. Cbh2. The present invention also encompasses an isolated polynucleotide comprising a nucleic acid that is 70%, 75%, or 80% identical, at least about 90% to about 95% identical, or at least about 96%, 97%, 98%, 99% or 100% identical to a nucleic acid encoding a Cochliobolus heterostrophus, Gibberella zeae, Irpex lacteus, Volvariella volvacea, or Piromyces sp. Cbh2 domain.

In some embodiments, the domain of the Cochliobolus heterostrophus, Gibberella zeae, Irpex lacteus, Volvariella volvacea, or Piromyces sp. Cbh2 is, for example, a GH6 family domain, a CBM domain or a signal peptide. In some specific embodiments, the domain is selected from the domains shown in Table 2 below.

TABLE 2
Exemplary Domains of Cbh2 Proteins.

| Organism & | Domain (amino acids) |
|---|---|
| Gene | Sequence |
| Cochliobolus | GH Family 6 Domain (aa 42-354) |
| heterostrophus | ANPYYSSEVHTLAMPSLPASLKPAATAVAKVGSFVWMDTMAKVPLMD |
| C4 ce17 | TYLADIKAKNAAGANLMGTFVVYDLPDRDCAALASNGELKIDEGGVEK |
| | YKTQYIDKIAAIIKKYPDVKINLAIEPDSLANMVINMGVQKCSRAAPYY |
| | KELTAYALKTLNENNVDMYMDGGHAGWLGWDANIGPTAKLFAEVYK |
| | AAGSPRGVRGIVTNVSNYNALRVSSCPSITQGNKNCDEERYINALAPLLK |
| | NEGFPAHFIVDQGRSGKVPTNQQEWGDWCNVSGAGEGTRPTTNTGNAL |
| | IDAIVWVKPGGESDGTSDTSAARYD |
| | (SEQ ID NO: 22) |
| | Signal Peptide (aa 1-18) |
| | MLSNVFLTAALAAGLAQA |
| | (SEQ ID NO: 23) |
| | CBM Domain |
| | N/A |
| Gibberella | GH Family 6 Domain (aa 111-423) |
| zeae K59 | ANNYYRSEVSTLAEPKLSGAMATAAAKVADVPSFQWMDTYDHISFMED |
| cel6 | SLADIRKANKAGGNYAGQFVVYDLPDRDCAAAASNGEYSLDKDGKNK |
| | YKAYIADQGILQDYSDTRIILVIEPDSLANMVTNMNVPKCANAASAYKE |
| | LTIHALKELNLPNVSMYIDAGHGGWLGWPANLPPAAQLYGQLYKDAGK |
| | PSRLRGLVTNVSNYNAWKLSSKPDYTESNPNYDEQKYIHALSPLLEQEG |
| | WPGAKFIVDQGRSGKQPTGQKAWGDWCNAPGTGFGLRPSANTGDALV |
| | DAFVWVKPGGESDGTSDTSAARYDY |
| | (SEQ ID NO: 24) |
| | Signal Peptide (aa 1-18) |
| | MTAYKLFLAAAFAATALA |
| | (SEQ ID NO: 25) |
| | CBM Domain (aa 31-59) |
| | VWSQCGGQNWSGTPCCTSGNKCVKVNDFY |
| | (SEQ ID NO: 26) |
| Irpex lacteus | GH Family 6 Domain (aa 107-419) |
| MC-2 cex3 | VDLWANNYYRSEVSTLALPKLSGAMATAAAKVADVPSFQWMDTYDHIS |
| | FMEDSLADIRKANKAGGNYAGQFVVYDLPDRDCAAAASNGEYSLDKD |
| | GKNKYKAYIADQGILQDYSDTRIILVIEPDSLANMVTNMNVPKCANAAS |
| | AYKELTIHALKELNLPNVSMYIDAGHGGWLGWPANLPPAAQLYGQLYK |
| | DAGKPSRLRGLVTNVSNYNAWKLSSKPDYTESNPNYDEQKYIHALSPLL |
| | EQEGWPGAKFIVDQGRSGKQPTGQKAWGDWCNAPGTGFGLRPSANTG |
| | DALVDAFVWVKPGGESDGTSDTSAA |
| | (SEQ ID NO: 27) |
| | Signal Peptide Domain (aa 1-20) |
| | MTAYKLFLAAAFAATALAAP |
| | (SEQ ID NO: 28) |
| | CBM Domain (aa 25-52) |
| | QSCSNGVWSQCGGQNWSGTPCCTSGNKC |
| | (SEQ ID NO: 29) |
| Volvariella | GH Family 6 Domain (aa 120-409) |
| volvacea | KALKVAQIPTFTWFDVIAKTSTLGDYLAEASALGKSSGKKYLVQIVVYD |
| cbhII-I | LPDRDCAALASNGEFSIANNGLNNYKGYIDQLVAQIKKYPDVRVVAVIE |
| | PDSLANLVTNLNVSKCANAQTAYKAGVTYALQQLNSVGVYMYLDAGH |
| | AGWLGWPANLNPAAQLFSQLYRDAGSPQYVRGLATNVANYNALSASSP |
| | DPVTQGNPNYDELHYINALAPALQSGGFPAHFIVDQGRSGVQNIRQQWG |
| | DWCNVKGAGFGQRPTLSTGSSLIDAIVWIKPGGECDGTTNTSSPRYDS |
| | (SEQ ID NO: 30) |
| | Signal Peptide Domain (aa 1-20) |
| | MSRFSALTALLLSLPLLAIA |
| | (SEQ ID NO: 31) |
| | CBM Domain (aa 25-52) |
| | YGQCGGNGWTGPKTCVSGATCTVINDWY |
| | (SEQ ID NO: 32) |
| Piromyces | GH Family 6 Domain (aa 138-457) |
| sp. E2 cel6A | INPYYTAEVDGAIAQISNASLRAKAEKMKEFSNAIWLDTIKNMNEWLEK |
| | NLKYALAEQNETGKTVLTVFVVYDLPGRDCHALASNGELLANDSDWA |
| | RYQSEYIDVIEEKLKTYKSQPVVLVVEPDSLANMVTNLDSTPACRDSEK |
| | YYMDGHAYLIKKLGVLPHVAMYLDIGHAFWLGWDDNRLKAGKVYSK |
| | VIQSGAPGNVRGFASNVANYTPWEDPTLSRGPDTEWNPCPDEKRYIEAM |
| | YKDFKSAGIKSVYFIDDTSRNGHKTDRTHPGEWCNQTGVGIGARPQANP |
| | ISGMDYLDAFYWVKPLGESDGYSDTTAVRYD |
| | (SEQ ID NO: 33) |
| | Signal Peptide Domain (aa 1-19) |
| | MKASIALTAIAALAANASA |
| | (SEQ ID NO: 34) |
| | CBM Domain (aa 21-55) |
| | CFSERLGYPCCRGNEVFYTDNDGDWGVENGNWCGI |
| | (SEQ ID NO: 35) |
| | CBM Domain (aa 62-98) |
| | TCWSQALGYPCCTSTSDVAYVDGDGNWGVENGNWCGI |
| | (SEQ ID NO: 36) |

The present invention also encompasses variants of the cbh2 genes, as described above. Variants can contain alterations in the coding regions, non-coding regions, or both. Examples are polynucleotide variants containing alterations which produce silent substitutions, additions, or deletions, but do not alter the properties or activities of the encoded polypeptide. In certain embodiments, nucleotide variants are produced by silent substitutions due to the degeneracy of the genetic code. In further embodiments, Cochliobolus heterostrophus, Gibberella zeae, Irpex lacteus, Volvariella volvacea, and Piromyces sp. cbh2 polynucleotide variants can be produced for a variety of reasons, e.g., to optimize codon expression for a particular host (e.g., change codons in the cbh2 mRNA to those preferred by a host such as the yeast Saccharomyces cerevisiae). Codon-optimized polynucleotides of the present invention are discussed further below.

The present invention also encompasses an isolated polynucleotide comprising a nucleic acid that is at least about 70%, 75%, or 80% identical, at least about 90% to about 95% identical, or at least about 96%, 97%, 98%, 99% or 100% identical to a nucleic acid encoding a fusion protein, wherein the nucleic acid comprises (1) a first polynucleotide, where the first polynucleotide encodes for a Cochliobolus heterostrophus, Gibberella zeae, Irpex lacteus, Volvariella volvacea, or Piromyces sp. Cbh2, or domain, fragment, variant, or derivative thereof; and (2) a second polynucleotide.

In certain embodiments, the second polynucleotide encodes for a CBM domain, for example, the CBM domain of T. reesei Cbh1 or T. reesei Cbh2. The second polynucleotide can also encode for the CBM domain of Gibberella zeae, Irpex lacteus, Volvariella volvacea, or Piromyces sp. Cbh2. For example, the first polynucleotide can encode for Cochliobolus heterostrophus Cbh2 or a fragment thereof, and the second polynucleotide can encode for the CBM domain of T. reesei Cbh1 or Gibberella zeae Cbh2. In addition, the first polynucleotide can encode for Gibberella zeae Cbh2 or a fragment thereof, and the second polynucleotide can encode for the CBM of Gibberella zeae Cbh2.

In further embodiments of the fusion polynucleotide, the first and second polynucleotides are in the same orientation, or the second polynucleotide is in the reverse orientation of the first polynucleotide. In additional embodiments, the first polynucleotide is either 5′ or 3′ to the second polynucleotide. In certain other embodiments, the first polynucleotide and/or the second polynucleotide are encoded by codon-optimized polynucleotides, for example, polynucleotides codon-optimized for expression in S. cerevisiae. In particular embodiments of the nucleic acid encoding a fusion protein, the first polynucleotide is a codon-optimized Cochliobolus heterostrophus, Gibberella zeae, Irpex Zacteus, Volvariella volvacea, or Piromyces sp. cbh2, and the second polynucleotide encodes for a codon-optimized CBM from T. reesei Cbh1 or Cbh2.

Also provided in the present invention are allelic variants, orthologs, and/or species homologs. Procedures known in the art can be used to obtain full-length genes, allelic variants, splice variants, full-length coding portions, orthologs, and/or species homologs of genes corresponding to any of SEQ ID NOs: 1-5, using information from the sequences disclosed herein or the clones deposited with the ATCC. For example, allelic variants and/or species homologs can be isolated and identified by making suitable probes or primers from the sequences provided herein and screening a suitable nucleic acid source for allelic variants and/or the desired homologue.

Polynucleotides comprising sequences that are at least 80%, 85%, 90%, 95%, 96%, 97%, 98%, or 99% identical to the entire sequence of any of SEQ ID NOs:1-5 or any fragment or domain therein can be used according to the methods described herein. Some embodiments of the invention encompass a nucleic acid molecule comprising at least 10, 20, 30, 35, 40, 50, 60, 70, 80, 90, 100, 200, 300, 400, 500, 600, 700, or 800 consecutive nucleotides or more of any of SEQ ID NOs:1-5, or domains, fragments, variants, or derivatives thereof.

The polynucleotide of the present invention can be in the form of RNA or in the form of DNA, which DNA includes cDNA, genomic DNA, and synthetic DNA. The DNA can be double stranded or single-stranded, and if single stranded can be the coding strand or non-coding (anti-sense) strand. The coding sequence which encodes the mature polypeptide can be identical to the coding sequence encoding SEQ ID NOs:11-15 or can be a different coding sequence which coding sequence, as a result of the redundancy or degeneracy of the genetic code, encodes the same mature polypeptide as the DNA of any one of SEQ ID NOs:1-5.

In certain embodiments, the present invention provides an isolated polynucleotide comprising a nucleic acid fragment which encodes at least 10, at least 20, at least 30, at least 40, at least 50, at least 60, at least 70, at least 80, at least 90, at least 95, or at least 100 or more contiguous amino acids of SEQ ID NOs:11-15.

The polynucleotide encoding for the mature polypeptide of SEQ ID NOs:11-15 can include: only the coding sequence for the mature polypeptide; the coding sequence of any domain of the mature polypeptide; or the coding sequence for the mature polypeptide (or domain-encoding sequence) together with non-coding sequence, such as introns or non-coding sequences 5′ and/or 3′ of the coding sequence for the mature polypeptide.

Thus, the term “polynucleotide encoding a polypeptide” encompasses a polynucleotide which includes only sequences encoding for the polypeptide as well as a polynucleotide which includes additional coding and/or non-coding sequences.

In further aspects of the invention, nucleic acid molecules having sequences with at least about 90%, 95%, 96%, 97%, 98% or 99% identity to the nucleic acid sequences disclosed herein, encode a polypeptide having Cbh2 functional activity. The phrase “a polypeptide having Cbh2 functional activity” is intended to refer to a polypeptide exhibiting activity similar, but not necessarily identical, to a functional activity of the Cbh2 polypeptides of the present invention, as measured, for example, in a particular biological assay. For example, a Cbh2 functional activity can routinely be measured by determining the ability of a Cbh2 polypeptide to hydrolyze cellulose, i.e. by measuring the level of Cbh2 activity.

Of course, due to the degeneracy of the genetic code, one of ordinary skill in the art will immediately recognize that a large portion of the nucleic acid molecules having a sequence at least about 90%, 95%, 96%, 97%, 98%, or 99% identical to the nucleic acid sequence of any of SEQ ID NOs:1-5, or fragments thereof, will encode polypeptides “having Cbh2 functional activity.” In fact, since degenerate variants of any of these nucleotide sequences all encode the same polypeptide, in many instances, this will be clear to the skilled artisan even without performing the above described comparison assay. It will be further recognized in the art that, for such nucleic acid molecules that are not degenerate variants, a reasonable number will also encode a polypeptide having Cbh2 functional activity.

Fragments of the full length gene of the present invention can be used as a hybridization probe for a cDNA library to isolate the full length cDNA and to isolate other cDNAs which have a high sequence similarity to the cbh1 genes of the present invention, or a gene encoding for a protein with similar biological activity. The probe length can vary from 5 bases to tens of thousands of bases, and will depend upon the specific test to be done. Typically a probe length of about 15 bases to about 30 bases is suitable. Only part of the probe molecule need be complementary to the nucleic acid sequence to be detected. In addition, the complementarity between the probe and the target sequence need not be perfect. Hybridization does occur between imperfectly complementary molecules with the result that a certain fraction of the bases in the hybridized region are not paired with the proper complementary base.

In certain embodiments, a hybridization probe can have at least 30 bases and can contain, for example, 50 or more bases. The probe can also be used to identify a cDNA clone corresponding to a full length transcript and a genomic clone or clones that contain the complete gene including regulatory and promoter regions, exons, and introns. An example of a screen comprises isolating the coding region of the gene by using the known DNA sequence to synthesize an oligonucleotide probe. Labeled oligonucleotides having a sequence complementary to that of the gene of the present invention are used to screen a library of bacterial or fungal cDNA, genomic DNA or mRNA to determine to which members of the library the probe hybridizes.

The present invention further relates to polynucleotides which hybridize to the hereinabove-described sequences if there is at least about 70%, at least about 90%, or at least about 95% identity between the sequences. The present invention particularly relates to polynucleotides which hybridize under stringent conditions to the hereinabove-described polynucleotides. As herein used, the term “stringent conditions” means hybridization will occur only if there is at least about 95% or at least about 97% identity between the sequences. In certain aspects of the invention, the polynucleotides which hybridize to the hereinabove described polynucleotides encode polypeptides which either retain substantially the same biological function or activity as the mature polypeptide encoded by the DNAs of any of SEQ ID NOs:1-5.

Alternatively, polynucleotides which hybridize to the hereinabove-described sequences can have at least 20 bases, at least 30 bases, or at least 50 bases which hybridize to a polynucleotide of the present invention and which has an identity thereto, as hereinabove described, and which may or may not retain activity. For example, such polynucleotides can be employed as probes for the polynucleotide of any of SEQ ID NOs: 1-5, for example, for recovery of the polynucleotide or as a diagnostic probe or as a PCR primer.

Hybridization methods are well defined and have been described above. Nucleic acid hybridization is adaptable to a variety of assay formats. One of the most suitable is the sandwich assay format. The sandwich assay is particularly adaptable to hybridization under non-denaturing conditions. A primary component of a sandwich-type assay is a solid support. The solid support has adsorbed to it or covalently coupled to it immobilized nucleic acid probe that is unlabeled and complementary to one portion of the sequence.

For example, genes encoding similar proteins or polypeptides to those of the instant invention could be isolated directly by using all or a portion of the instant nucleic acid fragments as DNA hybridization probes to screen libraries from any desired bacteria using methodology well known to those skilled in the art. Specific oligonucleotide probes based upon the instant nucleic acid sequences can be designed and synthesized by methods known in the art (see, e.g., Maniatis, 1989). Moreover, the entire sequences can be used directly to synthesize DNA probes by methods known to the skilled artisan such as random primers DNA labeling, nick translation, or end-labeling techniques, or RNA probes using available in vitro transcription systems.

In certain aspects of the invention, polynucleotides which hybridize to the hereinabove-described sequences having at least 20 bases, at least 30 bases, or at least 50 bases which hybridize to a polynucleotide of the present invention can be employed as PCR primers. Typically, in PCR-type amplification techniques, the primers have different sequences and are not complementary to each other. Depending on the desired test conditions, the sequences of the primers should be designed to provide for both efficient and faithful replication of the target nucleic acid. Methods of PCR primer design are common and well known in the art. Generally two short segments of the instant sequences can be used in polymerase chain reaction (PCR) protocols to amplify longer nucleic acid fragments encoding homologous genes from DNA or RNA. The polymerase chain reaction can also be performed on a library of cloned nucleic acid fragments wherein the sequence of one primer is derived from the instant nucleic acid fragments, and the sequence of the other primer takes advantage of the presence of the polyadenylic acid tracts to the 3′ end of the mRNA precursor encoding microbial genes. Alternatively, the second primer sequence can be based upon sequences derived from the cloning vector. For example, the skilled artisan can follow the RACE protocol (Frohman et al., PNAS USA 85:8998 (1988)) to generate cDNAs by using PCR to amplify copies of the region between a single point in the transcript and the 3′ or 5′ end. Primers oriented in the 3′ and 5′ directions can be designed from the instant sequences. Using commercially available 3′ RACE or 5′ RACE systems (BRL), specific 3′ or 5′ cDNA fragments can be isolated (Ohara et al., PNAS USA 86:5673 (1989); Loh et al., Science 243:217 (1989)).

In addition, specific primers can be designed and used to amplify a part of or the full-length of the instant sequences. The resulting amplification products can be labeled directly during amplification reactions or labeled after amplification reactions, and used as probes to isolate full length DNA fragments under conditions of appropriate stringency.

Therefore, the nucleic acid sequences and fragments thereof of the present invention can be used to isolate genes encoding homologous proteins from the same or other fungal species or bacterial species. Isolation of homologous genes using sequence-dependent protocols is well known in the art. Examples of sequence-dependent protocols include, but are not limited to, methods of nucleic acid hybridization, and methods of DNA and RNA amplification as exemplified by various uses of nucleic acid amplification technologies (e.g., polymerase chain reaction, Mullis et al., U.S. Pat. No. 4,683,202; ligase chain reaction (LCR) (Tabor, S. et al., Proc. Acad. Sci. USA 82, 1074, (1985)); or strand displacement amplification (SDA), (Walker, et al., Proc. Natl. Acad. Sci. USA 89, 392, (1992)).

The polynucleotides of the present invention also comprise nucleic acids encoding a Cochliobolus heterostrophus, Gibberella zeae, Irpex lacteus, Volvariella volvacea or Piromyces sp. Cbh2, or domain, fragment, variant, or derivative thereof, fused to a polynucleotide encoding a marker sequence which allows for selection and/or detection of the presence of the polynucleotide in an organism. Expression of the marker can be independent from expression of the Cbh2 polypeptide. The marker sequence can be a yeast selectable marker such as URA3, HIS3, LEU2, TRP1, LYS2, ADE2 or SMR1. See, e.g., Casey, G. P. et al., J. Inst. Brew. 94:93-97 (1988).

### Codon Optimization

As used herein the term “codon-optimized coding region” means a nucleic acid coding region that has been adapted for expression in the cells of a given organism by replacing one, or more than one, or a significant number, of codons with one or more codons that are more frequently used in the genes of that organism.

In general, highly expressed genes in an organism are biased towards codons that are recognized by the most abundant tRNA species in that organism. One measure of this bias is the “codon adaptation index” or “CAI,” which measures the extent to which the codons used to encode each amino acid in a particular gene are those which occur most frequently in a reference set of highly expressed genes from an organism. The Codon Adaptation Index is described in more detail in Sharp and Li, Nucleic Acids Research 15: 1281-1295 (1987)), which is incorporated by reference herein in its entirety.

The CAI of codon-optimized sequences of the present invention corresponds to from about 0.6 to about 1.0, from about 0.7 to about 1.0, from about 0.8 to about 1.0, from about 0.9 to about 1.0, from about 9.5 to about 1.0, or about 1.0. A codon-optimized sequence can be further modified for expression in a particular organism, depending on that organism's biological constraints. For example, large runs of “As” or “Ts” (e.g., runs greater than 4, 5, 6, 7, 8, 9, or 10 consecutive bases) can be removed from the sequences if these are known to effect transcription negatively. Furthermore, specific restriction enzyme sites can be removed for molecular cloning purposes. Examples of such restriction enzyme sites include Pad, AscI, BamHI, BglII, EcoRI and XhoI. Additionally, the DNA sequence can be checked for direct repeats, inverted repeats and mirror repeats with lengths of ten bases or longer, which can be modified manually by replacing codons with “second best” codons, i.e., codons that occur at the second highest frequency within the particular organism for which the sequence is being optimized.

Deviations in the nucleotide sequence that comprise the codons encoding the amino acids of any polypeptide chain allow for variations in the sequence coding for the gene. Since each codon consists of three nucleotides, and the nucleotides comprising DNA are restricted to four specific bases, there are 64 possible combinations of nucleotides, 61 of which encode amino acids (the remaining three codons encode signals ending translation). The “genetic code” which shows which codons encode which amino acids is reproduced herein as Table 3. As a result, many amino acids are designated by more than one codon. For example, the amino acids alanine and proline are coded for by four triplets, serine and arginine by six, whereas tryptophan and methionine are coded by just one triplet. This degeneracy allows for DNA base composition to vary over a wide range without altering the amino acid sequence of the proteins encoded by the DNA.

TABLE 3
The Standard Genetic Code.

| | T | C | A | G |
|---|---|---|---|---|
| T | TTT Phe (F) | TCT Ser (S) | TAT Tyr (Y) | TGT Cys (C) |
| | TTC Phe (F) | TCC Ser (S) | TAC Tyr (Y) | TGC |
| | TTA Leu (L) | TCA Ser (S) | TAA Ter | TGA Ter |
| | TTG Leu (L) | TCG Ser (S) | TAG Ter | TGG Trp (W) |
| C | CTT Leu (L) | CCT Pro (P) | CAT His (H) | CGT Arg (R) |
| | CTC Leu (L) | CCC Pro (P) | CAC His (H) | CGC Arg (R) |
| | CTA Leu (L) | CCA Pro (P) | CAA Gln (Q) | CGA Arg (R) |
| | CTG Leu (L) | CCG Pro (P) | CAG Gln (Q) | CGG Arg (R) |
| A | ATT Ile (I) | ACT Thr (T) | AAT Asn (N) | AGT Ser (S) |
| | ATC Ile (I) | ACC Thr (T) | AAC Asn (N) | AGC Ser (S) |
| | ATA Ile (I) | ACA Thr (T) | AAA Lys (K) | AGA Arg (R) |
| | ATG Met | ACG Thr (T) | AAG Lys (K) | AGG Arg (R) |
| | (M) | | | |
| G | GTT Val (V) | GCT Ala (A) | GAT Asp (D) | GGT Gly (G) |
| | GTC Val (V) | GCC Ala (A) | GAC Asp (D) | GGC Gly (G) |
| | GTA Val (V) | GCA Ala (A) | GAA Glu (E) | GGA Gly (G) |
| | GTG Val (V) | GCG Ala (A) | GAG Glu (E) | GGG Gly (G) |

Many organisms display a bias for use of particular codons to code for insertion of a particular amino acid in a growing peptide chain. Codon preference or codon bias, differences in codon usage between organisms, is afforded by degeneracy of the genetic code, and is well documented among many organisms. Codon bias often correlates with the efficiency of translation of messenger RNA (mRNA), which is in turn believed to be dependent on, inter alia, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization.

Given the large number of gene sequences available for a wide variety of animal, plant and microbial species, it is possible to calculate the relative frequencies of codon usage. Codon usage tables and codon-optimizing programs are readily available, for example, at http://phenotype.biosci.umbc.edu/codon/sgd/index.php (visited Sep. 4, 2009) or at http://www.kazusa.or.jp/codon/(visited Sep. 4, 2009), and these tables can be adapted in a number of ways. See Nakamura, Y., et al. “Codon usage tabulated from the international DNA sequence databases: status for the year 2000,” Nucl. Acids Res. 28:292 (2000). Codon usage tables for yeast, calculated from GenBank Release 128.0 [15 Feb. 2002], are reproduced below as Table 4. This table uses mRNA nomenclature, and so instead of thymine (T) which is found in DNA, the tables use uracil (U) which is found in RNA. The Table has been adapted so that frequencies are calculated for each amino acid, rather than for all 64 codons.

TABLE 4
Codon Usage Table for Saccharomyces cerevisiae Genes.

| | | | Frequency per |
|---|---|---|---|
| Amino Acid | Codon | Number | hundred |
| Phe | UUU | 170666 | 26.1 |
| Phe | UUC | 120510 | 18.4 |
| Leu | UUA | 170884 | 26.2 |
| Leu | UUG | 177573 | 27.2 |
| Leu | CUU | 80076 | 12.3 |
| Leu | CUC | 35545 | 5.4 |
| Leu | CUA | 87619 | 13.4 |
| Leu | CUG | 68494 | 10.5 |
| Ile | AUU | 196893 | 30.1 |
| Ile | AUC | 112176 | 17.2 |
| Ile | AUA | 116254 | 17.8 |
| Met | AUG | 136805 | 20.9 |
| Val | GUU | 144243 | 22.1 |
| Val | GUC | 76947 | 11.8 |
| Val | GUA | 76927 | 11.8 |
| Val | GUG | 70337 | 10.8 |
| Ser | UCU | 153557 | 23.5 |
| Ser | UCC | 92923 | 14.2 |
| Ser | UCA | 122028 | 18.7 |
| Ser | UCG | 55951 | 8.6 |
| Ser | AGU | 92466 | 14.2 |
| Ser | AGC | 63726 | 9.8 |
| Pro | CCU | 88263 | 13.5 |
| Pro | CCC | 44309 | 6.8 |
| Pro | CCA | 119641 | 18.3 |
| Pro | CCG | 34597 | 5.3 |
| Thr | ACU | 132522 | 20.3 |
| Thr | ACC | 83207 | 12.7 |
| Thr | ACA | 116084 | 17.8 |
| Thr | ACG | 52045 | 8.0 |
| Ala | GCU | 138358 | 21.2 |
| Ala | GCC | 82357 | 12.6 |
| Ala | GCA | 105910 | 16.2 |
| Ala | GCG | 40358 | 6.2 |
| Tyr | UAU | 122728 | 18.8 |
| Tyr | UAC | 96596 | 14.8 |
| His | CAU | 89007 | 13.6 |
| His | CAC | 50785 | 7.8 |
| Gln | CAA | 178251 | 27.3 |
| Gln | CAG | 79121 | 12.1 |
| Asn | AAU | 233124 | 35.7 |
| Asn | AAC | 162199 | 24.8 |
| Lys | AAA | 273618 | 41.9 |
| Lys | AAG | 201361 | 30.8 |
| Asp | GAU | 245641 | 37.6 |
| Asp | GAC | 132048 | 20.2 |
| Glu | GAA | 297944 | 45.6 |
| Glu | GAG | 125717 | 19.2 |
| Cys | UGU | 52903 | 8.1 |
| Cys | UGC | 31095 | 4.8 |
| Trp | UGG | 67789 | 10.4 |
| Arg | CGU | 41791 | 6.4 |
| Arg | CGC | 16993 | 2.6 |
| Arg | CGA | 19562 | 3.0 |
| Arg | CGG | 11351 | 1.7 |
| Arg | AGA | 139081 | 21.3 |
| Arg | AGG | 60289 | 9.2 |
| Gly | GGU | 156109 | 23.9 |
| Gly | GGC | 63903 | 9.8 |
| Gly | GGA | 71216 | 10.9 |
| Gly | GGG | 39359 | 6.0 |
| Stop | UAA | 6913 | 1.1 |
| Stop | UAG | 3312 | 0.5 |
| Stop | UGA | 4447 | 0.7 |

By utilizing this or similar tables, one of ordinary skill in the art can apply the frequencies to any given polypeptide sequence, and produce a nucleic acid fragment of a codon-optimized coding region which encodes the polypeptide, but which uses codons optimal for a given species. Codon-optimized coding regions can be designed by various different methods.

In one method, a codon usage table is used to find the single most frequent codon used for any given amino acid, and that codon is used each time that particular amino acid appears in the polypeptide sequence. For example, referring to Table 4 above, for leucine, the most frequent codon is UUG, which is used 27.2% of the time. Thus all the leucine residues in a given amino acid sequence would be assigned the codon UUG.

In another method, the actual frequencies of the codons are distributed randomly throughout the coding sequence. Thus, using this method for optimization, if a hypothetical polypeptide sequence had 100 leucine residues, referring to Table 4 for frequency of usage in the S. cerevisiae, about 5, or 5% of the leucine codons would be CUC, about 11, or 11% of the leucine codons would be CUG, about 12, or 12% of the leucine codons would be CUU, about 13, or 13% of the leucine codons would be CUA, about 26, or 26% of the leucine codons would be UUA, and about 27, or 27% of the leucine codons would be UUG.

These frequencies would be distributed randomly throughout the leucine codons in the coding region encoding the hypothetical polypeptide. As will be understood by those of ordinary skill in the art, the distribution of codons in the sequence will can vary significantly using this method; however, the sequence always encodes the same polypeptide.

Codon-optimized sequences of the present invention include those as set forth in Table 5 below.

TABLE 5
Cellobiohydrolase 2 (cbh2) polynucleotice sequences codon-optimized for expression in
S. cerevisiae.

Cochliobolus heterostrophus C4 cel7
ttaattaaaatgttgtctaacgtttttttgactgctgctttggctgctggtttggctcaagctttgccacaagctactccaactccaactgctgctcca
tctggtaatccatttgctggtaagaatttttacgctaacccatattattcttcagaagttcatactttggctatgccatctttgccagcttcattgaaac
cagctgctactgctgttgctaaagttggttcttttgtttggatggatactatggctaaagttccattgatggatacttacttggctgatattaaagcta
aaaatgctgctggtgctaatttgatgggtactttcgttgtttatgatttgccagatagagattgtgctgctttagcttctaatggtgaattgaaaattg
atgaaggtggtgttgaaaaatacaagacacaatacattgataagattgctgctattatcaaaaagtacccagatgttaagattaatttggctattg
aaccagattctttggctaatatggttactaatatgggtgttcaaaaatgttctagagctgctccatattacaaagaattgactgcttatgctttgaaa
actttgaacttcaacaacgttgacatgtatatggatggtggtcatgctggttggttgggttgggatgctaatattggtccaactgctaaattgtttg
ctgaagtttacaaagctgctggttctccaagaggtgttagaggtattgttacaaacgtttctaattacaacgctttgagagtttcttcttgtccatcta
ttactcaaggtaacaagaattgtgatgaagaaagatacattaatgctttggctccattgttgaaaaatgaaggttttccagctcattttattgttgat
caaggtagatcaggtaaagttccaactaatcaacaagaatggggtgattggtgtaatgtttctggtgctggttttggtactagaccaactactaa
tactggtaatgctttgattgatgctattgtttgggttaaaccaggtggtgaatctgatggtacttctgatacttctgctgcaagatatgatgctcattg
tggtagaaattctgcttttaaaccagctccagaagctggtacttggtttcaagcttactttgaaatgttgttgaagaatgctaatccagctttggcat
tataaggcgcgcc
(SEQ ID NO: 6)
Gibberella zeae K59 cel6
ttaattaaaatgactgcttacaaattgtttttggctgctgcttttgctgctactgctttggctgctccagttgaagaaagacaatcttgttctaatggtg
tttggtcacaatgtggtggtcaaaattggtctggtactccatgttgtacatctggtaacaagtgtgttaaggttaatgatttctactctcaatgtcaa
ccaggttctgctgatccatctccaacttctactattgtttctgctactactactaaagctactactacaggttctggtggttctgttacttctccacca
ccagttgctacaaacaatccattttctggtgttgatttgtgggcaaacaattattacagatcagaagtttctactttggctattccaaaattgtctggt
gctatggctactgctgctgcaaaagttgctgatgttccatcttttcaatggatggatacttacgatcatatttctttcatggaagattctttggctgat
attagaaaagcaaacaaagcaggtggtaattatgctggtcaattcgttgtttatgatttgccagatagagattgtgctgctgctgcttctaatggt
gaatactctttggataaagatggtaaaaacaagtacaaagcttatattgctgatcaaggtattttgcaagattactctgatactagaatcattttggt
tattgaaccagattctttagctaacatggttactaatatgaatgttccaaaatgtgctaatgctgcttctgcttacaaagaattgactattcatgcttt
gaaagaattgaatttgccaaacgtttcaatgtatattgatgctggtcatggtggttggttgggttggccagctaatttgccacctgctgctcaattg
tatggtcaattgtacaaagatgctggtaaaccatctagattgagaggtttggttactaatgtttctaattacaacgcttggaaattatcttctaagcc
agattatactgaatctaacccaaattacgatgaacaaaagtacattcatgctttatctccattgttggaacaagaaggttggccaggcgctaagt
tcattgttgatcaaggtagatcaggtaaacaaccaactggtcaaaaagcttggggtgattggtgtaatgctccaggtactggttttggtttaaga
ccatctgctaatactggtgatgctttggttgatgcttttgtttgggttaaaccaggtggtgaatctgatggtacttctgatacttctgctgcaagatat
gattatcattgtggtattgatggtgctgttaaaccagctccagaagctggtacttggtttcaagcttactttgaacaattgttgaagaatgctaatcc
atctttcttgttataaggcgcgcc
(SEQ ID NO: 7)
Irpex lacteus MC-2 cex3
ttaattaaaatgaagtctgctgcttttttggctgctttagctgctattttgccagcttacgttgctggtcaagctcaaacttgggctcaatgtggtggt
attggttttactggtccaactacttgtgttgctggttctgtttgtactaaacaaaacgattactactctcaatgtattccaggttctgctactactccaa
cttctgctccaacatctgcaccaacttctcaaccatcacaaccatcttctacttcatctgctccatctggtccatcttctacaccaactccatctgct
aacaatccatggactggttatcaaatttacttgtctccatactatgctaatgaagttgctgcagctgctaaagctattactgatccaactttggctg
ctaaagcagcttctgttgctaatattccaaatttcacttggttggattctgtttctaaaattgctgatttgaaaacttatttggctgatgcttctgctttg
ggtaaatcttctggtcaaaagcaattgttgcaaattgttgtttatgatttgccagatagagattgtgctgcaaaagcttctaatggtgaattttctatt
gctgataatggtttggctaactaccaaaactacattgatcaaattgttgctgctgttaaacaatttccagatgttagagttgttgctgttattgaacc
agattctttggctaatttggttacaaatttaaacgttcaaaagtgtgctaatgctaaatctacttacttgactgctgttaattatgctttgaagcaattat
cttctgttggtgtttatcaatatatggatgctggtcatgctggttggttgggttggccagctaatttaactccagctgctcaattgtttgctcaagttt
attctgatgctggtaaatctccattcattaagggtttggctactaatgttgctaattacaatgctttgtctgctgcttctccagatccaattactcaag
gtgatccaaattacgatgaaattcattacattaatgctttggctccagctttgcaatctgctggttttccagctacttttattgttgatcaaggtagatc
aggtcaacaaaatcatagacaacaatggggtgattggtgtaacattaaaggtgctggttttggtactagaccaactactaatactggttcttcttt
gattgattctattgtttgggttaaaccaggtggtgaatctgatggtacttctaattcttcatctccaagatttgattctacttgttctttgtctgatgctac
tcaaccagctccagaagctggtacttggtttcaagcttactttgaaactttggtttctaaagctaatccaccattgttataaggcgcgcc
(SEQ ID NO: 8)
Volvariella volvacea cbhII-I
ttaattaaaatgtctagattctctgctttgactgctttgttgttgtctttgccattgttggctattgctcaatctccattgtatggtcaatgtggtggtaat
ggttggactggtccaaaaacttgtgtttctggtgctacttgtactgttattaatgattggtattggcaatgtttgccaggtaatggtccaacttcttctt
ctccaacttctactccaactacaactactactactggtggtccacaaccaactgttccagctgctggtaatccatatactggttacgaaatttactt
gtctccatattatgctgctgaagctcaagctgctgctgctcaaatttctgatgctactcaaaaagctaaagctttgaaagttgctcaaattccaact
tttacttggtttgatgttattgctaaaacttctactttgggtgattatttggctgaagcttctgctttgggtaaatcttctggtaaaaagtacttggttca
aattgttgtttatgatttgccagatagagattgtgctgctttggcttctaatggtgaattttctattgctaacaacggtttgaacaattacaaaggttac
attgatcaattggttgcacaaattaagaaatacccagatgttagagttgttgctgttattgaaccagattctttggctaatttggttacaaatttgaac
gtttctaagtgtgctaatgctcaaactgcttacaaagctggtgttacttatgctttgcaacaattgaactctgttggtgtttacatgtatttggatgct
ggtcatgctggttggttgggttggccagctaatttgaatccagctgctcaattgttttctcaattgtatagagatgctggttctccacaatacgttag
aggtttggctactaatgttgctaattacaatgctttgtctgcttcttcaccagatccagttactcaaggtaatccaaattacgatgaattgcattacat
taatgctttggctccagctttgcaatctggtggttttccagctcattttattgttgatcaaggtagatcaggtgttcaaaacattagacaacaatggg
gtgattggtgtaatgttaaaggtgctggttttggtcaaagaccaactttatctactggttcttctttgattgatgctattgtttggattaaaccaggtg
gtgaatgtgatggtactactaatacatcttctccaagatatgattctcattgtggtttgtctgatgctactccaaatgctcctgaagctggtcaatgg
tttcaagcttactttgaaactttggttagaaatgcttctccaccattgttataaggcgcgcc
(SEQ ID NO: 9)
Piromyces sp. E2 cel6A
ttaattaaaatgaaggcttctattgctttgactgctattgctgctttggctgctaatgcttctgctgcttgtttttctgaaagattgggttatccatgttgt
agaggtaatgaagttttctacactgataatgatggtgattggggtgttgaaaatggtaattggtgtggtattggtggtgcttctgctactacttgttg
gtcacaagctttaggttacccttgttgtacttctacttctgatgttgcttacgttgatggtgacggtaactggggtgtcgaaaacggtaactggtgc
ggtataattgcaggtggtaattcttctaacaacaactctggttctactattaatgttggtgatgttactattggtaaccaatacactcatactggtaat
ccatttgctggtcataaattctttattaacccatactatactgctgaagttgatggtgctattgctcaaatttctaatgcttctttgagagctaaagctg
aaaagatgaaagaattttctaacgctatttggttggatactattaagaatatgaacgaatggttggaaaagaatttgaaatatgctttggctgaac
aaaatgaaactggtaagactgttttgacagtttttgttgtttatgatttgccaggtagagattgtcatgctttagcttctaatggtgaattgttggctaa
tgattctgattgggcaagatatcaatctgaatacattgatgttattgaagaaaagttgaaaacttacaagtctcaaccagttgttttggttgttgaac
cagattctttggctaatatggttacaaatttggattctactccagcttgtagagattctgaaaaatactatatggatggtcatgcttacttgattaaaa
agttgggtgttttgccacatgttgcaatgtatttggatattggtcatgctttttggttgggttgggatgataatagattgaaagctggtaaagtttact
ctaaggttattcaatctggtgctccaggtaatgttagaggttttgcttctaatgttgctaattatactccatgggaagatccaactttgtctagaggt
ccagatactgaatggaatccatgtccagatgaaaaaagatacattgaagcaatgtacaaagattttaagtctgctggtattaagtctgtttacttc
attgatgatacttctagaaatggtcataagactgatagaactcatccaggtgaatggtgtaatcaaacaggtgttggtattggtgctagaccaca
agctaatccaatttctggtatggattacttggatgctttttattgggttaaaccattgggtgaatctgatggttattctgatactactgctgtcagatat
gatggttattgtggtcatgctactgctatgaaaccagctcctgaagctggtcaatggtttcaaaaacatttcgaacaaggtttggaaaatgctaat
ccaccattgttataaggcgcgcc
(SEQ ID NO: 10)

When using the methods above, the term “about” is used precisely to account for fractional percentages of codon frequencies for a given amino acid. As used herein, “about” is defined as one amino acid more or one amino acid less than the value given. The whole number value of amino acids is rounded up if the fractional frequency of usage is 0.50 or greater, and is rounded down if the fractional frequency of use is 0.49 or less. Using again the example of the frequency of usage of leucine in human genes for a hypothetical polypeptide having 62 leucine residues, the fractional frequency of codon usage would be calculated by multiplying 62 by the frequencies for the various codons. Thus, 7.28 percent of 62 equals 4.51 UUA codons, or “about 5,” i.e., 4, 5, or 6 UUA codons, 12.66 percent of 62 equals 7.85 UUG codons or “about 8,” i.e., 7, 8, or 9 UUG codons, 12.87 percent of 62 equals 7.98 CUU codons, or “about 8,” i.e., 7, 8, or 9 CUU codons, 19.56 percent of 62 equals 12.13 CUC codons or “about 12,” i.e., 11, 12, or 13 CUC codons, 7.00 percent of 62 equals 4.34 CUA codons or “about 4,” i.e., 3, 4, or 5 CUA codons, and 40.62 percent of 62 equals 25.19 CUG codons, or “about 25,” i.e., 24, 25, or 26 CUG codons.

Randomly assigning codons at an optimized frequency to encode a given polypeptide sequence, can be done manually by calculating codon frequencies for each amino acid, and then assigning the codons to the polypeptide sequence randomly. Additionally, various algorithms and computer software programs are readily available to those of ordinary skill in the art. For example, the “EditSeq” function in the Lasergene Package, available from DNAstar, Inc., Madison, Wis., the backtranslation function in the VectorNTI Suite, available from InforMax, Inc., Bethesda, Md., and the “backtranslate” function in the GCG—Wisconsin Package, available from Accelrys, Inc., San Diego, Calif. In addition, various resources are publicly available to codon-optimize coding region sequences, e.g., the “backtranslation” function at http://www.entelechon.com/bioinformatics/backtranslation.php?lang=eng (visited Sep. 4, 2009). Constructing a rudimentary algorithm to assign codons based on a given frequency can also easily be accomplished with basic mathematical functions by one of ordinary skill in the art.

A number of options are available for synthesizing codon-optimized coding regions designed by any of the methods described above, using standard and routine molecular biological manipulations well known to those of ordinary skill in the art. In one approach, a series of complementary oligonucleotide pairs of 80-90 nucleotides each in length and spanning the length of the desired sequence are synthesized by standard methods. These oligonucleotide pairs are synthesized such that upon annealing, they form double stranded fragments of 80-90 base pairs, containing cohesive ends, e.g., each oligonucleotide in the pair is synthesized to extend 3, 4, 5, 6, 7, 8, 9, 10, or more bases beyond the region that is complementary to the other oligonucleotide in the pair. The single-stranded ends of each pair of oligonucleotides is designed to anneal with the single-stranded end of another pair of oligonucleotides. The oligonucleotide pairs are allowed to anneal, and approximately five to six of these double-stranded fragments are then allowed to anneal together via the cohesive single stranded ends, and then they ligated together and cloned into a standard bacterial cloning vector, for example, a TOPO® vector available from Invitrogen Corporation, Carlsbad, Calif. The construct is then sequenced by standard methods. Several of these constructs consisting of 5 to 6 fragments of 80 to 90 base pair fragments ligated together, i.e., fragments of about 500 base pairs, are prepared, such that the entire desired sequence is represented in a series of plasmid constructs. The inserts of these plasmids are then cut with appropriate restriction enzymes and ligated together to form the final construct. The final construct is then cloned into a standard bacterial cloning vector, and sequenced. Additional methods would be immediately apparent to the skilled artisan. In addition, gene synthesis is readily available commercially.

In certain embodiments, an entire polypeptide sequence, or fragment, variant, or derivative thereof is codon-optimized by any of the methods described herein. Various desired fragments, variants or derivatives are designed, and each is then codon-optimized individually. In addition, partially codon-optimized coding regions of the present invention can be designed and constructed. For example, the invention includes a nucleic acid fragment of a codon-optimized coding region encoding a polypeptide in which at least about 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, or 100% of the codon positions have been codon-optimized for a given species. That is, they contain a codon that is preferentially used in the genes of a desired species, e.g., a yeast species such as Saccharomyces cerevisiae, in place of a codon that is normally used in the native nucleic acid sequence.

In additional embodiments, a full-length polypeptide sequence is codon-optimized for a given species resulting in a codon-optimized coding region encoding the entire polypeptide, and then nucleic acid fragments of the codon-optimized coding region, which encode fragments, variants, and derivatives of the polypeptide are made from the original codon-optimized coding region. As would be well understood by those of ordinary skill in the art, if codons have been randomly assigned to the full-length coding region based on their frequency of use in a given species, nucleic acid fragments encoding fragments, variants, and derivatives would not necessarily be fully codon-optimized for the given species. However, such sequences are still much closer to the codon usage of the desired species than the native codon usage. The advantage of this approach is that synthesizing codon-optimized nucleic acid fragments encoding each fragment, variant, and derivative of a given polypeptide, although routine, would be time consuming and would result in significant expense.

The codon-optimized coding regions can be versions encoding a Cbh2 from Cochliobolus heterostrophus, Gibberella zeae, Irpex lacteus, Volvariella volvacea, or Piromyces sp. or domains, fragments, variants, or derivatives thereof.

Codon optimization is carried out for a particular species by methods described herein. For example, in certain embodiments codon-optimized coding regions encoding polypeptides of a Cochliobolus heterostrophus, Gibberella zeae, Irpex lacteus, Volvariella volvacea, or Piromyces sp. Cbh2, or domains, fragments, variants, or derivatives thereof are optimized according to yeast codon usage, e.g., Saccharomyces cerevisiae. In particular, the present invention relates to codon-optimized coding regions encoding polypeptides of a Cochliobolus heterostrophus, Gibberella zeae, Irpex lacteus, Volvariella volvacea, or Piromyces sp. Cbh2, or domains, variants, or derivatives thereof which have been optimized according to yeast codon usage, for example, Saccharomyces cerevisiae codon usage. Also provided are polynucleotides, vectors, and other expression constructs comprising codon-optimized coding regions encoding Cbh2 polypeptides of Cochliobolus heterostrophus, Gibberella zeae, Irpex lacteus, Volvariella volvacea, or Piromyces sp., or domains, fragments, variants, or derivatives thereof, and various methods of using such polynucleotides, vectors and other expression constructs.

In certain embodiments described herein, a codon-optimized coding region encoding the polypeptide sequence of any of SEQ ID NOs:11-15, or domains, fragments, variants, or derivatives thereof, is optimized according to codon usage in yeast (Saccharomyces cerevisiae). Alternatively, a codon-optimized coding region encoding the polypeptide sequence of any of SEQ ID NOs:11-15 can be optimized according to codon usage in any plant, animal, or microbial species.

### Cbh2 Polypeptides

The present invention further relates to the expression of Cochliobolus heterostrophus, Gibberella zeae, Irpex lacteus, Volvariella volvacea, or Piromyces sp. Cbh2 polypeptides in a host cell, such as Saccharomyces cerevisiae. The sequences of Cochliobolus heterostrophus, Gibberella zeae, Irpex lacteus, Volvariella volvacea, and Piromyces sp. Cbh2 polypeptides are set forth in Table 6 below.

TABLE 6
Cellobiohydrolase 2 (Cbh2) polypeptide sequences.

Cochliobolus heterostrophus C4 cel7 (GenBank AAM76664.1)
MLSNVFLTAALAAGLAQALPQATPTPTAAPSGNPFAGKNFYANPYYSSEVHTLAMPSLP
ASLKPAATAVAKVGSFVWMDTMAKVPLMDTYLADIKAKNAAGANLMMTFVVYDLPD
RDCAALASNGELKIDEGGVEKYKTQYIDKIAAIIKKYPDVKINLAIEPDSLANMVTNMGV
QKCSRAAPYYKELTAYALKTLNFNNVDMYMDGGHAGWLGWDANIGPTAKLFAEVYK
AAGSPRGVRGIVTNVSNYNALRVSSCPSITQGNKNCDEERYINALAPLLKNEGFPAHFIV
DQGRSGKVPTNQQEWGDWCNVSGAGFGTRPTTNTGNALIDAIVWVKPGGESDGTSDTS
AARYDAHCGRNSAFKPAPEAGTWFQAYFEMLLKNANPALA
(SEQ ID NO: 11)
Gibberella zeae K59 cel6 (GenBank Accession AY302753.1)
MTAYKLFLAAAFAATALAAPVEERQSCSNGVWSQCGGQNWSGTPCCTSGNKCVKVND
FYSQCQPGSADPSPTSTIVSATTTKATTTGSGGSVTSPPPVATNNPFSGVDLWANNYYRS
EVSTLAIPKLSGAMATAAAKVADVPSFQWMDTYDHISFMEDSLADIRKANKAGGNYAG
QFVVYDLPDRDCAAAASNGEYSLDKDGKNKYKAYIADQGILQDYSDTRIILVIEPDSLA
NMVTNMNVPKCANAASAYKELTIHALKELNLPNVSMYIDAGHGGWLGWPANLPPAAQ
LYGQLYKDAGIUSRLRGLVTNVSNYNAWKLSSKPDYTESNPNYDEQKYIHALSPLLEQE
GWPGAKFIVDQGRSGKQPTGQKAWGDWCNAPGTGFGLRPSANTGDALVDAFVWYKP
GGESDGTSDTSAARYDYHCGIDGAVKPAPEAGTWFQAYFEQLLKNANPSFL
(SEQ ID NO: 12)
Irpex lacteus MC-2 cex3 (GenBank Accession BAG48183.1)
MTAYKLFLAAAFAATALAAPVEERQSCSNGVWSQCGGQNWSGTPCCTSGNKCVKVND
FYSQCQPGSADPSPTSTIVSATTTKATTTGSGGSVTSPPPVATNNPFSGVDLWANNYYRS
EVSTLAIPKLSGAMATAAAKVADVPSFQWMDTYDHISFMEDSLADIRKANKAGGNYAG
QFVVYDLPDRDCAAAASNGEYSLDKDGKNKYKAYIADQGILQDYSDTRIILVIEPDSLA
NMVTNMNVPKCANAASAYKELTIHALKELNLPNVSMYIDAGHGGWLGWPANLPPAAQ
LYGQLYKDAGIUSRLRGLVTNVSNYNAWKLSSKPDYTESNPNYDEQKYIHALSPLLEQE
GWPGAKFIVDQGRSGKQPTGQKAWGDWCNAPGTGFGLRPSANTGDALVDAFVWVKP
GGESDGTSDTSAARYDYHCGIDGAVKPAPEAGTWFQAYFEQLLKNANPSFL
(SEQ ID NO: 13)
Volvariella volvacea cbhII-I (GenBank Accession AAT64008.1)
MSRFSALTALLLSLPLLAIAQSPLYGQCGGNGWTGPKTCVSGATCTVINDWYWQCLPG
NGPTSSSPTSTPTTTTTTGGPQPTVPAAGNPYTGYEIYLSPYYAAEAQAAAAQISDATQK
AKALKVAQIPTFTWFDVIAKTSTLGDYLAEASALGKSSGKKYLVQIVVYDLPDRDCAAL
ASNGEFSIANNGLNNYKGYIDQLVAQIKKYPDVRVVAVIEPDSLANLVTNLNVSKCANA
QTAYKAGVTYALQQLNSVGVYMYLDAGHAGWLGWPANLNPAAQLFSQLYRDAGSPQ
YVRGLATNVANYNALSASSPDPVTQGNPNYDELHYINALAPALQSGGFPAHFIVDQGRS
GVQNIRQQWGDWCNVKGAGFGQRPTLSTGSSLIDAIVWIKPGGECDGTTNTSSPRYDSH
CGLSDATPNAPEAGQWFQAYFETLVRNASPPL
(SEQ ID NO: 14)
Piromyces sp. E2 cel6A (GenBank Accession AAL92497.1)
MKASIALTAIAALAANASAACFSERLGYPCCRGNEVFYTDNDGDWGVENGNWCGIGGA
SATTCWSQALGYPCCTSTSDVAYVDGDGNWGVENGNWCGIIAGGNSSNNNSGSTINVG
DVTIGNQYTHTGNPFAGHKFFINPYYTAEVDGAIAQISNASLRAKAEKMKEFSNAIWLDT
IKNMNEWLEKNLKYALAEQNETGKTVLTVEVVYDLPGRDCHALASNGELLANDSDWA
RYQSEYIDVIEEKLKTYKSQPVVLVVEPDSLANMVTNLDSTPACRDSEKYYMDGHAYLI
KKLGVLPHVAMYLDIGHAFWLGWDDNRLKAGKVYSKVIQSGAPGNVRGFASNVANYT
PWEDPTLSRGPDTEWNPCPDEKRYIEAMYKDFKSAGIKSVYFIDDTSRNGHKTDRTHPG
EWCNQTGVGIGARPQANPISGMDYLDAFYWVKPLGESDGYSDTTAVRYDGYCGHATA
MKPAPEAGQWFQKHFEQGLENANPPL
(SEQ ID NO: 15)

The present invention further encompasses polypeptides which comprise, or alternatively consist of, an amino acid sequence which is at least about 80%, 85%, 90%, 95%, 96%, 97%, 98%, or 99% identical to, for example, the polypeptide sequences shown in any of SEQ ID NOs: 11-15, and/or domains, fragments, variants, or derivative thereof, of any of these polypeptides (e.g., those fragments described herein, or domains of any of SEQ ID NOs: 11-15).

By a polypeptide having an amino acid sequence at least, for example, 95% “identical” to a query amino acid sequence of the present invention, it is intended that the amino acid sequence of the subject polypeptide is identical to the query sequence except that the subject polypeptide sequence can include up to five amino acid alterations per each 100 amino acids of the query amino acid sequence. In other words, to obtain a polypeptide having an amino acid sequence at least 95% identical to a query amino acid sequence, up to 5% of the amino acid residues in the subject sequence can be inserted, deleted, (indels) or substituted with another amino acid. These alterations of the reference sequence can occur at the amino or carboxy terminal positions of the reference amino acid sequence or anywhere between those terminal positions, interspersed either individually among residues in the reference sequence or in one or more contiguous groups within the reference sequence.

As a practical matter, whether any particular polypeptide is at least 80%, 85%, 90%, 95%, 96%, 97%, 98% or 99% identical to, for instance, any of the amino acid sequences of SEQ ID NOs: 11-15 can be determined conventionally using known computer programs. As discussed above, a method for determining the best overall match between a query sequence (a sequence of the present invention) and a subject sequence, also referred to as a global sequence alignment, can be determined using the FASTDB computer program based on the algorithm of Brutlag et al. (Comp. App. Biosci. 6:237-245 (1990)). In a sequence alignment the query and subject sequences are either both nucleotide sequences or both amino acid sequences. The result of said global sequence alignment is in percent identity. Preferred parameters used in a FASTDB amino acid alignment are: Matrix=PAM 0, k-tuple=2, Mismatch Penalty=1, Joining Penalty=20, Randomization Group Length=0, Cutoff Score=1, Window Size=sequence length, Gap Penalty=5, Gap Size Penalty=0.05, Window Size=500 or the length of the subject amino acid sequence, whichever is shorter. Also as discussed above, manual corrections can be made to the results in certain instances.

In certain aspects of the invention, the polypeptides and polynucleotides of the present invention are provided in an isolated form, e.g., purified to homogeneity.

The present invention also encompasses polypeptides which comprise, or alternatively consist of, an amino acid sequence which is at least about 80%, 85%, 90%, 95%, 96%, 97%, 98%, or 99% similar to the polypeptide of any of SEQ ID NOs: 11-15, or to portions of such polypeptide, wherein the portion can contain at least 30 amino acids, at least 50 amino acids, at least 100 amino acids, at least 150 amino acids, at least 200 amino acids, at least 250 amino acids, at least 300 amino acids, or at least 350 amino acids.

As known in the art “similarity” between two polypeptides is determined by comparing the amino acid sequence and conserved amino acid substitutes thereto of the polypeptide to the sequence of a second polypeptide.

The present invention further relates to a domain, fragment, variant, derivative, or analog of the polypeptide of any of SEQ ID NOs: 11-15.

Fragments or portions of the polypeptides of the present invention can be employed for producing the corresponding full-length polypeptide by peptide synthesis, therefore, the fragments can be employed as intermediates for producing the full-length polypeptides.

Fragments of Cbh2 polypeptides of the present invention can encompass domains, proteolytic fragments, and deletion fragments of Cochliobolus heterostrophus, Gibberella zeae, Irpex lacteus, Volvariella volvacea, or Piromyces sp. Cbh2 polypeptides. The fragments can optionally retain a specific biological activity of the Cbh2 protein. Exemplary fragments include those described in Table 2 above. Polypeptide fragments further include any portion of the polypeptide which comprises a catalytic activity of the Cbh2 protein.

The variant, derivative or analog of the polypeptide of any of SEQ ID NOs: 11-15, can be (i) one in which one or more of the amino acid residues are substituted with a conserved or non-conserved amino acid residue and such substituted amino acid residue may or may not be one encoded by the genetic code, or (ii) one in which one or more of the amino acid residues includes a substituent group, or (iii) one in which the mature polypeptide is fused with another compound, such as a compound to increase the half-life of the polypeptide (for example, polyethylene glycol), or (iv) one in which the additional amino acids are fused to the mature polypeptide for purification of the polypeptide or (v) one in which a fragment of the polypeptide is soluble, i.e., not membrane bound, yet still binds ligands to the membrane bound receptor. Such variants, derivatives and analogs are deemed to be within the scope of those skilled in the art from the teachings herein.

The polypeptides of the present invention further include variants of the polypeptides. A “variant” of the polypeptide can be a conservative variant, or an allelic variant. As used herein, a conservative variant refers to alterations in the amino acid sequence that does not adversely affect the biological functions of the protein. A substitution, insertion or deletion is said to adversely affect the protein when the altered sequence prevents or disrupts a biological function associated with the protein. For example, the overall charge, structure or hydrophobic-hydrophilic properties of the protein can be altered without adversely affecting a biological activity. Accordingly, the amino acid sequence can be altered, for example to render the peptide more hydrophobic or hydrophilic, without adversely affecting the biological activities of the protein.

By an “allelic variant” is intended alternate forms of a gene occupying a given locus on a chromosome of an organism. Genes II, Lewin, B., ed., John Wiley & Sons, New York (1985). Non-naturally occurring variants can be produced using art-known mutagenesis techniques. Allelic variants, though possessing a slightly different amino acid sequence than those recited above, will still have the same or similar biological functions associated with the Cochliobolus heterostrophus, Gibberella zeae, Irpex lacteus, Volvariella volvacea, or Piromyces sp. Cbh2 protein.

The allelic variants, the conservative substitution variants, and members of the Cbh2 protein family, will have an amino acid sequence having at least 75%, at least 80%, at least 90%, at least 95%, at least 96%, at least 97%, at least 98%, or at least 99% amino acid sequence identity with a Cochliobolus heterostrophus, Gibberella zeae, Irpex lacteus, Volvariella volvacea, or Piromyces sp. Cbh2 amino acid sequence set forth in any one of SEQ ID NOs:11-15. Identity or homology with respect to such sequences is defined herein as the percentage of amino acid residues in the candidate sequence that are identical with the known peptides, after aligning the sequences and introducing gaps, if necessary, to achieve the maximum percent homology, and not considering any conservative substitutions as part of the sequence identity. N terminal, C terminal, or internal extensions, deletions, or insertions into the peptide sequence shall not be construed as affecting homology.

Thus, the proteins and peptides of the present invention include molecules comprising the amino acid sequence of any one of SEQ ID NOs: 11-15 or fragments thereof having a consecutive sequence of at least about 3, 4, 5, 6, 10, 15, 20, 25, 30, 35, 50, 100, 150, 200, 250, 300, 350, or more amino acid residues of the Cochliobolus heterostrophus, Gibberella zeae, Irpex lacteus, Volvariella volvacea, or Piromyces sp. Cbh2 polypeptide sequence; amino acid sequence variants of such sequences wherein at least one amino acid residue has been inserted N- or C terminal to, or within, the disclosed sequence; amino acid sequence variants of the disclosed sequences, or their fragments as defined above, that have been substituted by another residue. Contemplated variants further include those containing predetermined mutations by, e.g., homologous recombination, site-directed or PCR mutagenesis, and the corresponding proteins of other organisms, the alleles or other naturally occurring variants of the family of proteins; and derivatives wherein the protein has been covalently modified by substitution, chemical, enzymatic, or other appropriate means with a moiety other than a naturally occurring amino acid (for example, a detectable moiety such as an enzyme or radioisotope).

Using known methods of protein engineering and recombinant DNA technology, variants can be generated to improve or alter the characteristics of the CBH polypeptides. For instance, one or more amino acids can be deleted from the N-terminus or C-terminus of the secreted protein without substantial loss of biological function.

Thus, the invention further includes Cochliobolus heterostrophus, Gibberella zeae, Irpex Zacteus, Volvariella volvacea, or Piromyces sp. Cbh2 polypeptide variants which show substantial biological activity. Such variants include deletions, insertions, inversions, repeats, and substitutions selected according to general rules known in the art so as have little effect on activity.

The skilled artisan is fully aware of amino acid substitutions that are either less likely or not likely to significantly effect protein function (e.g., replacing one aliphatic amino acid with a second aliphatic amino acid), as further described below.

For example, guidance concerning how to make phenotypically silent amino acid substitutions is provided in Bowie et al., “Deciphering the Message in Protein Sequences: Tolerance to Amino Acid Substitutions,” Science 247:1306-1310 (1990), wherein the authors indicate that there are two main strategies for studying the tolerance of an amino acid sequence to change.

The first strategy exploits the tolerance of amino acid substitutions by natural selection during the process of evolution. By comparing amino acid sequences in different species, conserved amino acids can be identified. These conserved amino acids are likely important for protein function. In contrast, the amino acid positions where substitutions have been tolerated by natural selection indicates that these positions are not critical for protein function. Thus, positions tolerating amino acid substitution could be modified while still maintaining biological activity of the protein.

The second strategy uses genetic engineering to introduce amino acid changes at specific positions of a cloned gene to identify regions critical for protein function. For example, site directed mutagenesis or alanine-scanning mutagenesis (introduction of single alanine mutations at every residue in the molecule) can be used. See, e.g., Cunningham and Wells, Science 244:1081-1085 (1989). The resulting mutant molecules can then be tested for biological activity.

As the authors state, these two strategies have revealed that proteins are often surprisingly tolerant of amino acid substitutions. The authors further indicate which amino acid changes are likely to be permissive at certain amino acid positions in the protein. For example, most buried (within the tertiary structure of the protein) amino acid residues require nonpolar side chains, whereas few features of surface side chains are generally conserved. Moreover, tolerated conservative amino acid substitutions involve replacement of the aliphatic or hydrophobic amino acids Ala, Val, Leu and Ile; replacement of the hydroxyl residues Ser and Thr; replacement of the acidic residues Asp and Glu; replacement of the amide residues Asn and Gln, replacement of the basic residues Lys, Arg, and His; replacement of the aromatic residues Phe, Tyr, and Trp, and replacement of the small-sized amino acids Ala, Ser, Thr, Met, and Gly.

The terms “derivative” and “analog” refer to a polypeptide differing from the Cochliobolus heterostrophus, Gibberella zeae, Irpex lacteus, Volvariella volvacea, or Piromyces sp. Cbh2 polypeptide, but retaining essential properties thereof. Generally, derivatives and analogs are overall closely similar, and, in many regions, identical to the Cochliobolus heterostrophus, Gibberella zeae, Irpex lacteus, Volvariella volvacea, or Piromyces sp. Cbh2 polypeptide. The term “derivative” and “analog” when referring to Cochliobolus heterostrophus, Gibberella zeae, Irpex lacteus, Volvariella volvacea, and Piromyces sp. Cbh2 polypeptides of the present invention include polypeptides which retain at least some of the activity of the corresponding native polypeptide, e.g., the exoglucanase activity.

Derivatives of Cochliobolus heterostrophus, Gibberella zeae, Irpex lacteus, Volvariella volvacea, or Piromyces sp. Cbh2 polypeptides of the present invention, are polypeptides which have been altered so as to exhibit additional features not found on the native polypeptide. Derivatives can be covalently modified by substitution, chemical, enzymatic, or other appropriate means with a moiety other than a naturally occurring amino acid (for example, a detectable moiety such as an enzyme or radioisotope). Examples of derivatives include fusion proteins.

An analog is another form of a Cochliobolus heterostrophus, Gibberella zeae, Irpex lacteus, Volvariella volvacea, or Piromyces sp. Cbh2 polypeptide of the present invention. An “analog” also retains substantially the same biological function or activity as the polypeptide of interest, i.e., functions as a cellobiohydrolase. An analog includes a proprotein which can be activated by cleavage of the proprotein portion to produce an active mature polypeptide.

The polypeptide of the present invention can be a recombinant polypeptide, a natural polypeptide, or a synthetic polypeptide.

### Tethered and Secreted Cbh2 Polypeptides

According to the present invention, the Cochliobolus heterostrophus, Gibberella zeae, Irpex lacteus, Volvariella volvacea, or Piromyces sp. Cbh2 polypeptides can be either tethered or secreted. As used herein, a protein is “tethered” to an organism's cell surface if at least one terminus of the protein is bound, covalently and/or electrostatically for example, to the cell membrane or cell wall. It will be appreciated that a tethered protein can include one or more enzymatic regions that can be joined to one or more other types of regions at the nucleic acid and/or protein levels (e.g., a promoter, a terminator, an anchoring domain, a linker, a signaling region, etc.). While the one or more enzymatic regions may not be directly bound to the cell membrane or cell wall (e.g., such as when binding occurs via an anchoring domain), the protein is nonetheless considered a “tethered enzyme” according to the present specification.

Tethering can, for example, be accomplished by incorporation of an anchoring domain into a recombinant protein that is heterologously expressed by a cell, or by prenylation, fatty acyl linkage, glycosyl phosphatidyl inositol anchors or other suitable molecular anchors which can anchor the tethered protein to the cell membrane or cell wall of the host cell. A tethered protein can be tethered at its amino terminal end or optionally at its carboxy terminal end.

As used herein, “secreted” means released into the extracellular milieu, for example into the media. Although tethered proteins can have secretion signals as part of their immature amino acid sequence, they are maintained as attached to the cell surface, and do not fall within the scope of secreted proteins as used herein.

As used herein, “flexible linker sequence” refers to an amino acid sequence which links two amino acid sequences, for example, a cell wall anchoring amino acid sequence with an amino acid sequence that contains the desired enzymatic activity. The flexible linker sequence allows for necessary freedom for the amino acid sequence that contains the desired enzymatic activity to have reduced steric hindrance with respect to proximity to the cell and can also facilitate proper folding of the amino acid sequence that contains the desired enzymatic activity.

In some embodiments of the present invention, the tethered Cochliobolus heterostrophus, Gibberella zeae, Irpex lacteus, Volvariella volvacea, or Piromyces sp. Cbh2 polypeptides are tethered by a flexible linker sequence linked to an anchoring domain. In some embodiments, the anchoring domain is the anchoring domain of CWP2 (for carboxy terminal anchoring) or FLO1 (for amino terminal anchoring) from S. cerevisiae.

In some embodiments, heterologous secretion signals can be added to the expression vectors of the present invention to facilitate the extra-cellular expression of cellulase proteins. In some embodiments, the heterologous secretion signal is the secretion signal from T. reesei Xyn2.

### Cbh2 Fusion Polypeptides

The present invention also encompasses fusion proteins comprising two or more polypeptides. For example, the fusion proteins can be a fusion of a Cochliobolus heterostrophus, Gibberella zeae, Irpex lacteus, Volvariella volvacea, or Piromyces sp. Cbh2 and a second peptide. The Cbh2 and the second peptide can be fused directly or indirectly, for example, through a linker sequence. The fusion protein can comprise for example, a second peptide that is N-terminal to the Cbh2 and/or a second peptide that is C-terminal to the heterologous cellulase. Thus, in certain embodiments, the polypeptide of the present invention comprises a first polypeptide and a second polypeptide, wherein the first polypeptide comprises a Cochliobolus heterostrophus, Gibberella zeae, Irpex lacteus, Volvariella volvacea, or Piromyces sp. Cbh2 polypeptide.

According to the present invention, the fusion protein can comprise a first and second polypeptide wherein the first polypeptide comprises a Cochliobolus heterostrophus, Gibberella zeae, Irpex lacteus, Volvariella volvacea, or Piromyces sp. Cbh2 polypeptide and the second polypeptide comprises a signal sequence. The signal sequence can be from any organism. For example, in some embodiments, the second polypeptide is an S. cerevisiae polypeptide. In one particular embodiment, the S. cerevisiae polypeptide is the S. cerevisiae alpha mating factor signal sequence. In some embodiments the signal sequence comprises the amino acid sequence MRFPSIFTAVLFAASSALA (SEQ ID NO: 16).

According to another embodiment, the fusion protein can comprise a first and second polypeptide, wherein the first polypeptide comprises a Cochliobolus heterostrophus, Gibberella zeae, Irpex lacteus, Volvariella volvacea, or Piromyces sp. Cbh2 polypeptide and the second polypeptide comprises a polypeptide used to facilitate purification or identification or a reporter peptide. The polypeptide used to facilitate purification or identification or the reporter peptide can be, for example, a HIS-tag, a GST-tag, an HA-tag, a FLAG-tag, a MYC-tag, or a fluorescent protein.

According to yet another embodiment, the fusion protein can comprise a first and second polypeptide, wherein the first polypeptide comprises a Cbh2 and the second polypeptide comprises an anchoring peptide. In some embodiments, the anchoring domain is the anchoring domain of CWP2 (for carboxy terminal anchoring) or FLO1 (for amino terminal anchoring) from S. cerevisiae.

According to yet another embodiment, the fusion protein can comprise a first and second polypeptide, wherein the first polypeptide comprises a Cochliobolus heterostrophus, Gibberella zeae, Irpex lacteus, Volvariella volvacea, or Piromyces sp. Cbh2 and the second polypeptide comprises a cellulose binding module (CBM). In some embodiments, the CBM is from Neosartorya fischeri Cbh1, H. grisea Cbh1, Chaetomium thermophilum Cbh1, T. reesei Cbh1 or T. reesei Cbh2, or a domain, fragment, variant, or derivative thereof.

In certain other embodiments, the first polypeptide and the second polypeptide are fused via a linker sequence. The linker sequence can, in some embodiments, be encoded by a codon-optimized polynucleotide. (Codon-optimized polynucleotides are described in more detail herein.) An amino acid sequence corresponding to a codon-optimized linker 1 according to the invention is a flexible linker-strep tag-TEV site-FLAG-flexible linker fusion and corresponds to GGGGSGGGGS AWHPQFGG ENLYFQG DYKDDDK GGGGSGGGGS (SEQ ID NO: 17).

The DNA sequence encoding the polypeptide of SEQ ID NO:17 is:

(SEQ ID NO: 18)
GGAGGAGGTGGTTCAGGAGGTGGTGGGTCTGCTTGGCATCCACAATTTGG
AGGAGGCGGTGGTGAAAATCTGTATTTCCAGGGAGGCGGAGGTGATTACA
AGGATGACGACAAAGGAGGTGGTGGATCAGGAGGTGGTGGCTCC

An amino acid sequence corresponding to optimized linker 2 is a flexible linker-strep tag-linker-TEV site-flexible linker and corresponds to GGGGSGGGGS WSHPQFEK GG ENLYFQG GGGGSGGGGS (SEQ ID NO: 19). The DNA sequence is as follows:

(SEQ ID NO: 20)
ggtggcggtggatctggaggaggcggttcttggtctcacccacaatttgaaaagggtggagaaaacttgtactttcaaggcggtggtggag
gttctggcggaggtggctccggctca.

In further embodiments of the fusion protein, the first and second polypeptide are in the same orientation, or the second polypeptide is in the reverse orientation of the first polypeptide. In additional embodiments, the first polypeptide is either N-terminal or C-terminal to the second polypeptide. In certain other embodiments, the first polypeptide and/or the second polypeptide are encoded by codon-optimized polynucleotides, for example, polynucleotides codon-optimized for S. cerevisiae.

### Vectors and Host Cells

The present invention also relates to vectors which include polynucleotides of the present invention, host cells which are genetically engineered with vectors of the invention and the production of polypeptides of the invention by recombinant techniques.

Host cells are genetically engineered (transduced or transformed or transfected) with the vectors of this invention which can be, for example, a cloning vector or an expression vector. The vector can be, for example, in the form of a plasmid, a viral particle, a phage, etc. The engineered host cells can be cultured in conventional nutrient media modified as appropriate for activating promoters, selecting transformants or amplifying the genes of the present invention. The culture conditions, such as temperature, pH and the like, are those previously used with the host cell selected for expression, and will be apparent to the ordinarily skilled artisan.

The polynucleotides of the present invention can be employed for producing polypeptides by recombinant techniques. Thus, for example, the polynucleotide can be included in any one of a variety of expression vectors for expressing a polypeptide. Such vectors include chromosomal, nonchromosomal and synthetic DNA sequences, e.g., derivatives of SV40; bacterial plasmids; and yeast plasmids. Such vectors also include “suicide vectors” which are not self-replicating but can be replicated after insertion into the host chromosome. Other vectors can also be used.

The appropriate DNA sequence can be inserted into the vector by a variety of procedures. In general, the DNA sequence is inserted into an appropriate restriction endonuclease site(s) by procedures known in the art. Such procedures and others are deemed to be within the scope of those skilled in the art.

The DNA sequence in the expression vector is operatively associated with an appropriate expression control sequence(s) (promoter) to direct mRNA synthesis. Representative examples of such promoters are as follows:

TABLE 7
Promoters.

| Gene | Organism | Systematic name | Reason for use/benefits |
|---|---|---|---|
| PGK1 | S. cerevisiae | YCR012W | Strong constitutive promoter |
| ENO1 | S. cerevisiae | YGR254W | Strong constitutive promoter |
| TDH3 | S. cerevisiae | YGR192C | Strong constitutive promoter |
| TDH2 | S. cerevisiae | YJR009C | Strong constitutive promoter |
| TDH1 | S. cerevisiae | YJL052W | Strong constitutive promoter |
| ENO2 | S. cerevisiae | YHR174W | Strong constitutive promoter |
| GPM1 | S. cerevisiae | YKL152C | Strong constitutive promoter |
| TPI1 | S. cerevisiae | YDR050C | Strong constitutive promoter |

Additional the E. coli, lac or trp, and other promoters known to control expression of genes in prokaryotic or lower eukaryotic cells. The expression vector also contains a ribosome binding site for translation initiation and a transcription terminator. The vector can also include appropriate sequences for amplifying expression, or can include additional regulatory regions.

In addition, the expression vectors can contain one or more selectable marker genes to provide a phenotypic trait for selection of transformed host cells such as URA3, HIS3, LEU2, TRP1, LYS2 or ADE2, dihydrofolate reductase or neomycin (G418) resistance or zeocin resistance for eukaryotic cell culture, or chloramphenicol, thiamphenicol, streptomycin, tetracycline, kanamycin, hygromycin, phleomycin or ampicillin resistance in E. coli.

The vector containing the appropriate DNA sequence as herein, as well as an appropriate promoter or control sequence, can be employed to transform an appropriate host to permit the host to express the protein.

Thus, in certain aspects, the present invention relates to host cells containing the above-described constructs. The host cell can be a higher eukaryotic cell, such as a mammalian cell, or a lower eukaryotic cell, such as a yeast cell, e.g., Saccharomyces cerevisiae, or the host cell can be a prokaryotic cell, such as a bacterial cell.

Representative examples of appropriate hosts include, for example, bacterial cells, such as E. coli, Streptomyces, Salmonella typhimurium; thermophilic or mesophilic bacteria; fungal cells, such as yeast; and plant cells, etc. The selection of an appropriate host is deemed to be within the scope of those skilled in the art from the teachings herein.

Appropriate fungal hosts include yeast. In certain aspects of the invention the yeast is Saccharomyces cerevisiae, Saccharomyces pastorianus (also known as Saccharomyces carlsbergensis), Saccharomyces bayanus, Kluyveromyces lactis, Kluyveromyces marxianus, Schizosaccharomyces pombe, Candida albicans, Pichia pastoris, Pichia stipitis, Yarrowia lipolytica, Hansenula polymorpha, Phaffia rhodozyma, Candida utilis, Arxula adeninivorans, Debaryomyces hansenii, Debaryomyces polymorphus or Schwanniomyces occidentalis. In some embodiments, the host cell can be an oleaginous yeast cell. In some particular embodiments, the oleaginous yeast cell is a Blakeslea, Candida, Cryptococcus, Cunninghamella, Lipomyces, Mortierella, Mucor, Phycomces, Pythium, Rhodosporidium, Rhodotorula, Trichosporon or Yarrowia cell.

According to the methods described herein, the yeast strains can be modified, e.g. to improve growth, selection, and/or stability. Thus, for example, the Saccharomyces cerevisiae, Saccharomyces pastorianus (also known as Saccharomyces carlsbergensis), Saccharomyces bayanus, Kluyveromyces lactis, Kluyveromyces marxianus, Schizosaccharomyces pombe, Candida albicans, Pichia pastoris, Pichia stipitis, Yarrowia lipolytica, Hansenula polymorpha, Phaffia rhodozyma, Candida utilis, Arxula adeninivorans, Debaryomyces hansenii, Debaryomyces polymorphus or Schwanniomyces occidentalis can include deletions, insertions, and/or rearrangements and still be considered Saccharomyces cerevisiae, Saccharomyces pastorianus (also known as Saccharomyces carlsbergensis), Saccharomyces bayanus, Kluyveromyces lactis, Kluyveromyces marxianus, Schizosaccharomyces pombe, Candida albicans, Pichia pastoris, Pichia stipitis, Yarrowia lipolytica, Hansenula polymorpha, Phaffia rhodozyma, Candida utilis, Arxula adeninivorans, Debaryomyces hansenii, Debaryomyces polymorphus or Schwanniomyces occidentalis. Derivatives of the aforementioned yeast cells, i.e., yeast that have been adapated sufficiently to diverge the genome to the extent that it is a different species can also be used according to the present methods. Thus, the host cells described herein include derivatives of Saccharomyces cerevisiae, Saccharomyces pastorianus (also known as Saccharomyces carlsbergensis), Saccharomyces bayanus, Kluyveromyces lactis, Kluyveromyces marxianus, Schizosaccharomyces pombe, Candida albicans, Pichia pastoris, Pichia stipitis, Yarrowia lipolytica, Hansenula polymorpha, Phaffia rhodozyma, Candida utilis, Arxula adeninivorans, Debaryomyces hansenii, Debaryomyces polymorphus and Schwanniomyces occidentalis.

More particularly, the present invention also includes recombinant constructs comprising one or more of the sequences as broadly described above. The constructs comprise a vector, such as a plasmid or viral vector, into which a sequence of the invention has been inserted, in a forward or reverse orientation. In one aspect of this embodiment, the construct further comprises regulatory sequences, including, for example, a promoter, operably associated to the sequence. Large numbers of suitable vectors and promoters are known to those of skill in the art, and are commercially available. The following vectors are provided by way of example.

Yeast: Yeast vectors include those of five general classes, based on their mode of replication in yeast, YIp (yeast integrating plasmids), YRp (yeast replicating plasmids), YCp (yeast replicating plasmids with centromere (CEN) elements incorporated), YEp (yeast episomal plasmids), and YLp (yeast linear plasmids). With the exception of the YLp plasmids, all of these plasmids can be maintained in E. coli as well as in Saccharomyces cerevisiae and thus are also referred to as yeast shuttle vectors. In certain aspects, these plasmids contain two types of selectable genes: plasmid-encoded drug-resistance genes and cloned yeast genes, where the drug resistant gene is typically used for selection in bacterial cells and the cloned yeast gene is used for selection in yeast. Drug-resistance genes include ampicillin, kanamycin, tetracycline, neomycin and sulfometuron methyl. Cloned yeast genes include HISS, LEU2, LYS2, TRP1, URA3, TRP1 and SMR1. pYAC vectors can also be utilized to clone large fragments of exogenous DNA on to artificial linear chromosomes.

In certain aspects of the invention, YCp plasmids, which have high frequencies of transformation and increased stability to due the incorporated centromere elements, are utilized. In certain other aspects of the invention, YEp plasmids, which provide for high levels of gene expression in yeast, are utilized. In additional aspects of the invention, YRp plasmids are utilized.

In certain embodiments, the vector comprises (1) a first polynucleotide, where the first polynucleotide encodes for a Cochliobolus heterostrophus, Gibberella zeae, Irpex lacteus, Volvariella volvacea, or Piromyces sp. Cbh2, or domain, fragment, variant, or derivative thereof; and (2) a second polynucleotide, where the second polynucleotide encodes for a Cochliobolus heterostrophus, Gibberella zeae, Irpex lacteus, Volvariella volvacea, or Piromyces sp. Cbh2, or domain, fragment, variant, or derivative thereof.

In certain additional embodiments, the vector comprises a first polynucleotide encoding for a Cochliobolus heterostrophus, Gibberella zeae, Irpex lacteus, Volvariella volvacea, or Piromyces sp. Cbh2 and a second polynucleotide encoding for the CBM domain of T. reesei CBH1 or T. reesei Cbh2. In further embodiments, the first and second polynucleotides are in the same orientation, or the second polynucleotide is in the reverse orientation of the first polynucleotide. In additional embodiments, the first polynucleotide is either N-terminal or C-terminal to the second polynucleotide. In certain other embodiments, the first polynucleotide and/or the second polynucleotide are encoded by codon-optimized polynucleotides, for example, polynucleotides codon-optimized for S. cerevisiae.

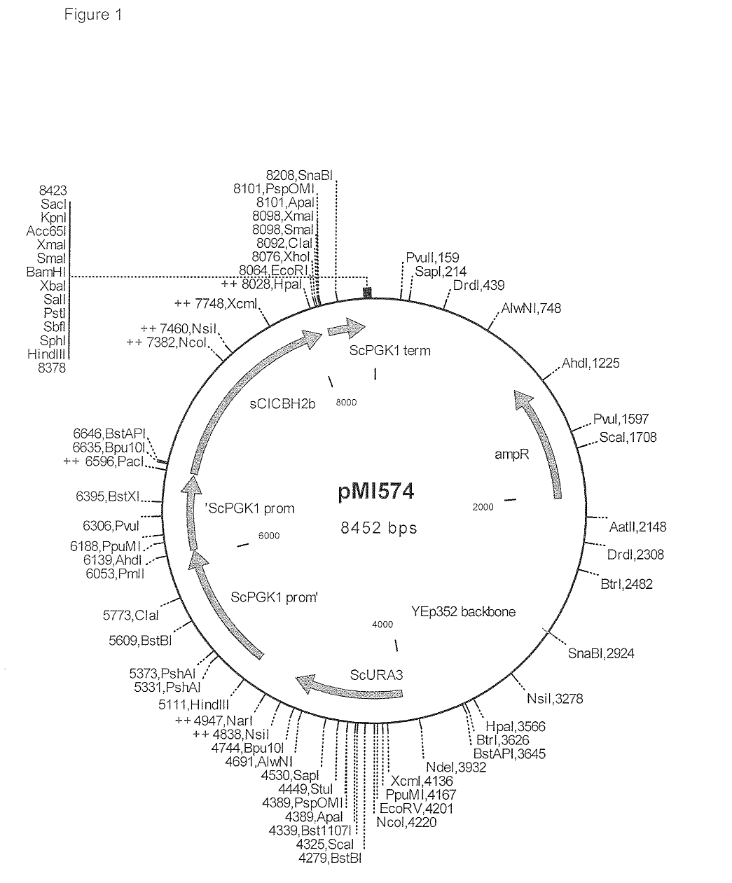

In particular embodiments, the vector of the present invention is a plasmid selected from the group consisting of pRDH150, pRDH151, pRDH152, pRDH153, and pRDH154. Descriptions of these plasmids are found in Example 1 and FIG. 1. However, any other plasmid or vector can be used as long as they are replicable and viable in the host.

Promoter regions can be selected from any desired gene. Particular named yeast promoters include the ENO1 promoter, the PGK1 promoter, the TEF1 promoter, and the HXT7 promoter. Particular named bacterial promoters include lacI, lacZ, T3, T7, gpt, lambda PR, PL and trp. Eukaryotic promoters include CMV immediate early, HSV thymidine kinase, early and late SV40, LTRs from retrovirus, and mouse metallothionein-I. Selection of the appropriate vector and promoter is well within the level of ordinary skill in the art.

Introduction of the construct into a host yeast cell, e.g., Saccharomyces cerevisiae, can be effected by lithium acetate transformation, spheroplast transformation, or transformation by electroporation, as described, for example, in Current Protocols in Molecular Biology, 13.7.1-13.7.10.

Introduction of the construct in other host cells can be effected by calcium phosphate transfection, DEAE-Dextran mediated transfection, or electroporation. See e.g., Davis, L., et al., Basic Methods in Molecular Biology, (1986).

The constructs in host cells can be used in a conventional manner to produce the gene product encoded by the recombinant sequence. Alternatively, the polypeptides of the invention can be synthetically produced by conventional peptide synthesizers.

Following creation of a suitable host cell and growth of the host cell to an appropriate cell density, the selected promoter is induced by appropriate means (e.g., temperature shift or chemical induction) and cells are cultured for an additional period.

Cells are typically harvested by centrifugation, disrupted by physical or chemical means, and the resulting crude extract retained for further purification.

Microbial cells employed in expression of proteins can be disrupted by any convenient method, including freeze-thaw cycling, sonication, mechanical disruption, or use of cell lysing agents, such methods are well know to those skilled in the art.

Yeast cells, e.g., Saccharomyces cerevisiae, employed in expression of proteins can be manipulated as follows. The Cbh2 polypeptides can be secreted by cells and therefore can be easily recovered from supernatant using methods known to those of skill in the art. Proteins can also be recovered and purified from recombinant cell cultures by methods including spheroplast preparation and lysis, cell disruption using glass beads, and cell disruption using liquid nitrogen, for example.

Various mammalian cell culture systems can also be employed to express recombinant protein. Expression vectors will comprise an origin of replication, a suitable promoter and enhancer, and also any necessary ribosome binding sites, polyadenylation site, splice donor and acceptor sites, transcriptional termination sequences, and 5′ flanking nontranscribed sequences.

Additional methods include ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Protein refolding steps can be used, as necessary, in completing configuration of the mature protein. Finally, high performance liquid chromatography (HPLC) can be employed for final purification steps.

The Cbh2 polypeptides can be prepared in any suitable manner. Such polypeptides include isolated naturally occurring polypeptides, recombinantly produced polypeptides, synthetically produced polypeptides, or polypeptides produced by a combination of these methods. Means for preparing such polypeptides are well understood in the art.

Cbh2 polypeptides are provided in an isolated form, and, in certain aspects, are substantially purified. A recombinantly produced version of a Cbh2 polypeptide, including the secreted polypeptide, can be substantially purified using techniques described herein or otherwise known in the art, such as, for example, by the one-step method described in Smith and Johnson, Gene 67:31-40 (1988). Cbh2 polypeptides also can be purified from natural, synthetic or recombinant sources using techniques described herein or otherwise known in the art.

The Cbh2 polypeptides of the present invention can be in the form of the secreted protein, including the mature form, or can be a part of a larger protein, such as a fusion protein. It can be advantageous to include an additional amino acid sequence which contains secretory or leader sequences, pro-sequences, sequences which aid in purification, such as multiple histidine residues, or an additional sequence for stability during recombinant production.

Secretion of desired proteins into the growth media has the advantages of simplified and less costly purification procedures. It is well known in the art that secretion signal sequences are often useful in facilitating the active transport of expressible proteins across cell membranes. The creation of a transformed host capable of secretion can be accomplished by the incorporation of a DNA sequence that codes for a secretion signal which is functional in the host production host. Methods for choosing appropriate signal sequences are well known in the art (see for example EP 546049; WO 9324631). The secretion signal DNA or facilitator can be located between the expression-controlling DNA and the instant gene or gene fragment, and in the same reading frame with the latter.

### Heterologous Expression of Cbh2 Polypeptides in Host Cells

In order to address the limitations of the previous systems, the present invention provides Cochliobolus heterostrophus, Gibberella zeae, Irpex lacteus, Volvariella volvacea, and Piromyces sp. Cbh2 polypeptides, and domains, variants, and derivatives thereof that can be effectively and efficiently utilized in a consolidated bioprocessing system.

In particular, the invention relates to the production of a heterologous Cbh2 in a host organism. In certain embodiments, this host organism is yeast, such as Saccharomyces cerevisiae.

In certain embodiments of the present invention, a host cell comprising a vector which encodes and expresses a Cochliobolus heterostrophus, Gibberella zeae, Irpex lacteus, Volvariella volvacea, or Piromyces sp. Cbh2 that is utilized for consolidated bioprocessing is co-cultured with additional host cells expressing one or more additional endoglucanases, cellobiohydrolases and/or β-glucosidases. In other embodiments of the invention, a host cell transformed with a plasmid encoding Cochliobolus heterostrophus, Gibberella zeae, Irpex lacteus, Volvariella volvacea, or Piromyces sp. Cbh2 additionally expresses one or more heterologous endoglucanases, cellobiohydrolases or β-glucosidases. The endoglucanase, cellobiohydrolase and/or β-glucosidase can be any suitable endoglucanase, cellobiohydrolase and β-glucosidase derived from, for example, a fungal or bacterial source.

In certain embodiments of the invention, the endoglucanase(s) can be an endoglucanase I or an endoglucanase II isoform, paralogue or orthologue. In another embodiment, the endoglucanase expressed by the host cells of the present invention can be recombinant endo-1,4-β-glucanase. In certain embodiments of the present invention, the endoglucanase is an endoglucanase I from Trichoderma reesei. In certain other embodiments of the invention, the endogluconase is C. formosanus endoglucanase I.

In certain embodiments of the present invention, the β-glucosidase is derived from Saccharomycopsis fibuligera. In certain embodiments, the β-glucosidase is a β-glucosidase I or a β-glucosidase II isoform, paralogue or orthologue. In certain other embodiments, the β-glucosidase expressed by the cells of the present invention can be recombinant β-glucanase I from a Saccharomycopsis fibuligera source.

In certain embodiments of the invention, the cellobiohydrolase(s) can be a cellobiohydrolase I and/or a cellobiohydrolase II isoform, paralogue or orthologue. In certain embodiments of the present invention the cellobiohydrolases are Trichoderma reesei Cbh1 or Cbh2, T. emersonii Cbh1 or Cbh2, or C. lucknowense cellobiohydrase IIb.

The transformed host cells or cell cultures, as described above, are measured for endoglucanase, cellobiohydrolase and/or β-glucosidase protein content. For the use of secreted cellulases, protein content can be determined by analyzing the host (e.g., yeast) cell supernatants. Proteins, including tethered heterologous biomass degrading enzymes, can also be recovered and purified from recombinant cell cultures by methods including spheroplast preparation and lysis, cell disruption using glass beads, and cell disruption using liquid nitrogen for example. Additional protein purification methods include trichloroacetic acid, ammonium sulfate or ethanol precipitation, acid extraction, anion or cation exchange chromatography, phosphocellulose chromatography, hydrophobic interaction chromatography, affinity chromatography, hydroxylapatite chromatography, gel filtration, and lectin chromatography. Protein refolding steps can be used, as necessary, in completing configuration of the mature protein. Finally, high performance liquid chromatography (HPLC) can be employed for final purification steps.

Protein analysis methods include methods such as the traditional Lowry method, the bicinchoninic acid protein assay reagent (Pierce) or the protein assay method according to BioRad's manufacturer's protocol. Using such methods, the protein content of saccharolytic enzymes can be estimated. Additionally, to accurately measure protein concentration a Cbh2 can be expressed with a tag, for example a His-tag or HA-tag and purified by standard methods using, for example, antibodies against the tag, a standard nickel resin purification technique or similar approach.

The transformed host cells or cell cultures, as described above, can be further analyzed for hydrolysis of cellulase (e.g., by a sugar detection assay), for a particular type of cellulase activity (e.g., by measuring the individual endoglucanase, cellobiohydrolase or β-glucosidase activity) or for total cellulase activity. Endoglucanase activity can be determined, for example, by measuring an increase of reducing ends in an endogluconase specific CMC substrate. Cellobiohydrolase activity can be measured, for example, by using insoluble cellulosic substrates such as the amorphous substrate phosphoric acid swollen cellulose (PASC) or microcrystalline cellulose (Avicel) and determining the extent of the substrate's hydrolysis. β-glucosidase activity can be measured by a variety of assays, e.g., using cellobiose.

A total cellulase activity, which includes the activity of endoglucanase, cellobiohydrolase I and cellobiohydrolase II, and β-glucosidase, can hydrolyze crystalline cellulose synergistically. Total cellulase activity can thus be measured using insoluble substrates including pure cellulosic substrates such as Whatman No. 1 filter paper, cotton linter, microcrystalline cellulose, bacterial cellulose, algal cellulose, and cellulose-containing substrates such as dyed cellulose, alpha-cellulose or pretreated lignocellulose.

It will be appreciated that suitable lignocellulosic material can be any feedstock that contains soluble and/or insoluble cellulose, where the insoluble cellulose can be in a crystalline or non-crystalline form. In various embodiments, the lignocellulosic biomass comprises, for example, wood, corn, corn cobs, corn stover, corn fiber, sawdust, bark, leaves, agricultural and forestry residues, grasses such as switchgrass, cord grass, rye grass or reed canary grass, miscanthus, ruminant digestion products, municipal wastes, paper mill effluent, newspaper, cardboard, miscanthus, sugar-processing residues, sugarcane bagasse, agricultural wastes, rice straw, rice hulls, barley straw, cereal straw, wheat straw, canola straw, oat straw, oat hulls, stover, soybean stover, forestry wastes, recycled wood pulp fiber, paper sludge, sawdust, hardwood, softwood or combinations thereof.

In certain embodiments of the present invention, a host cell comprising a vector which encodes and expresses a Cochliobolus heterostrophus, Gibberella zeae, Irpex lacteus, Volvariella volvacea, or Piromyces sp. Cbh2 that is utilized for consolidated bioprocessing is co-cultured with additional host cells expressing one or more additional heterologous endoglucanases, cellobiohydrolases and/or β-glucosidases. In other embodiments of the invention, a host cell transformed with a Cochliobolus heterostrophus, Gibberella zeae, Irpex lacteus, Volvariella volvacea, and Piromyces sp. CHB2 is transformed with and/or expresses one or more other heterologous endoglucanases, exogluconases or β-glucosidases. The endogluconase, exogluconase and/or β-glucosidase can be any suitable endogluconase, exogluconase and β-glucosidase.

Specific activity of cellulases can also be detected by methods known to one of ordinary skill in the art, such as by the Avicel assay (described supra) that would be normalized by protein (cellulase) concentration measured for the sample. To accurately measure protein concentration a Cochliobolus heterostrophus, Gibberella zeae, Irpex lacteus, Volvariella volvacea, or Piromyces sp. Cbh2 can be expressed with a tag, for example a His-tag or HA-tag and purified by standard methods using, for example, antibodies against the tag, a standard nickel resin purification technique or similar approach.

In some embodiments the host cell comprises a heterologous cellobiohydrolase that has a specific activity of at least about 0.20%, at least about 0.25%, at least about 0.30%, at least about 0.35%, or at least about 0.40%, Avicel hydrolysis per μg cellobiohydrolase per 48 hours based on an initial 1% Avicel concentration. In some embodiments, the host cell comprises a heterologous cellobiohydrolase that has a specific activity of from about 0.20% to about 0.90%, from about 0.20% to about 0.80%, from about 0.20% to about 0.70%, from about 0.20% to about 0.60%, from about 0.20% to about 0.50%, or from about 0.20% to about 0.45% Avicel hydrolysis per μg cellobiohydrolase per 48 hours based on an initial 1% Avicel concentration. In some embodiments, the host cell comprises a heterologous cellobiohydrolase that has a specific activity of from about 0.30% to about 0.90%, from about 0.30% to about 0.80%, from about 0.30% to about 0.70%, from about 0.30% to about 0.50%, or from about 0.30% to about 0.45% Avicel hydrolysis per μg cellobiohydrolase per 48 hours, based on an initial 1% Avicel concentration. In some embodiments, the host cell comprises a heterologous cellobiohydrolase that has a specific activity of from about 0.40% to about 0.90%, from about 0.40% to about 0.80%, from about 0.40% to about 0.70%, from about 0.40% to about 0.50%, or from about 0.40% to about 0.45% Avicel hydrolysis per μg cellobiohydrolase per 48 hours, based on an initial 1% Avicel concentration.

In some embodiments, the host cell comprises a heterologous cellobiohydrolase that has a specific activity on Avicel of at least about 0.08 μmol/mg/min, at least about 0.09 μmol/mg/min, at least about 0.10 μmol/mg/min, at least about 0.11 μmol/mg/min, at least about 0.12 μmol/mg/min, at least about 0.13 μmol/mg/min, at least about 0.14 μmol/mg/min, at least about 0.15 μmol/mg/min, or at least about 0.16 μmol/mg/min. In some embodiments, the host cell comprises a heterologous cellobiohydrolase that has a specific activity on Avicel from about 0.08 μmol/mg/min to about 0.30 μmol/mg/min, from about 0.08 μmol/mg/min to about 0.25 μmol/mg/min, or from about 0.08 μmol/mg/min to about 0.20 μmol/mg/min. In some embodiments, the host cell comprises a heterologous cellobiohydrolase that has a specific activity on Avicel from about 0.10 μmol/mg/min to about 0.30 μmol/mg/min, from about 0.10 μmol/mg/min to about 0.25 μmol/mg/min, or from about 0.10 μmol/mg/min to about 0.20 μmol/mg/min. In some embodiments, the host cell comprises a heterologous cellobiohydrolase that has a specific activity on Avicel from about 0.15 μmol/mg/min to about 0.30 μmol/mg/min, from about 0.15 μmol/mg/min to about 0.25 μmol/mg/min, or from about 0.15 μmol/mg/min to about 0.20 μmol/mg/min.

In additional embodiments, the transformed host cells or cell cultures are assayed for ethanol production. Ethanol production can be measured by techniques known to one or ordinary skill in the art. For example, the quantity of ethanol in fermentation samples can be assessed using HPLC analysis. Many ethanol assay kits are commercially available that use, for example, alcohol oxidase enzyme based assays. Methods of determining ethanol production are within the scope of those skilled in the art from the teachings herein.

### Co-Cultures

The present invention is also directed to co-cultures comprising at least two yeast host cells wherein the at least one yeast host cell comprises a polynucleotide encoding a Cochliobolus heterostrophus, Gibberella zeae, Irpex lacteus, Volvariella volvacea, or Piromyces sp. Cbh2 polypeptide and at least one other yeast host cell comprises a polynucleotide encoding a heterologous cellulase. As used herein, “co-culture” refers to growing two different strains or species of host cells together in the same vessel. In some embodiments of the invention, at least one host cell of the co-culture comprises a heterologous polynucleotide comprising a nucleic acid which encodes an endoglucanase, at least one host cell of the co-culture comprises a heterologous polynucleotide comprising a nucleic acid which encodes a β-glucosidase and at least one host cell comprises a heterologous polynucleotide comprising a nucleic acid which encodes a Cochliobolus heterostrophus, Gibberella zeae, Irpex lacteus, Volvariella volvacea, or Piromyces sp. Cbh2 polypeptide. In a further embodiment, the co-culture further comprises a host cell comprising a heterologous polynucleotide comprising a nucleic acid which encodes a second cellobiohydrolase.

The co-culture can comprise two or more strains of yeast host cells and the heterologous cellulases can be expressed in any combination in the two or more strains of host cells. For example, according to the present invention, the co-culture can comprise two strains: one strain of host cells that expresses an endoglucanase and a second strain of host cells that expresses a β-glucosidase, a Cochliobolus heterostrophus, Gibberella zeae, Irpex lacteus, Volvariella volvacea, or Piromyces sp. Cbh2 polypeptide and a second cellobiohydrolase. According to the present invention, the co-culture can also comprise four strains: one strain of host cells which expresses an endoglucanase, one strain of host cells that expresses a β-glucosidase, one strain of host cells which expresses a Cochliobolus heterostrophus, Gibberella zeae, Irpex lacteus, Volvariella volvacea, or Piromyces sp. Cbh2 polypeptide, and one strain of host cells which expressess a second cellobiohydrolase. Similarly, the co-culture can comprise one strain of host cells that expresses two cellulases, for example an endoglucanase and a beta-glucosidase and a second strain of host cells that expresses one or more cellobiohydrolases including one Cochliobolus heterostrophus, Gibberella zeae, Irpex lacteus, Volvariella volvacea, or Piromyces sp. Cbh2 polypeptide. The co-culture can, in addition to the at least one yeast host cell comprising a polynucleotide encoding a Cochliobolus heterostrophus, Gibberella zeae, Irpex lacteus, Volvariella volvacea, or Piromyces sp. Cbh2 polypeptide and at least one other yeast host cell comprising a polynucleotide encoding a heterologous cellulase, also include other host cells which do not comprise heterologous cellulases.

The various host cell strains in the co-culture can be present in equal numbers, or one strain or species of host cell can significantly outnumber another second strain or species of host cells. For example, in a co-culture comprising two strains or species of host cells the ratio of one host cell to another can be about 1:1, 1:2, 1:3, 1:4, 1:5, 1:10, 1:100, 1:500 or 1:1000. Similarly, in a co-culture comprising three or more strains or species of host cells, the strains or species of host cells can be present in equal or unequal numbers.

The co-cultures of the present invention can include tethered cellulases, secreted cellulases or both tethered and secreted cellulases. For example, in some embodiments of the invention, the co-culture comprises at least one yeast host cell comprising a polynucleotide encoding a secreted Cochliobolus heterostrophus, Gibberella zeae, Irpex lacteus, Volvariella volvacea, or Piromyces sp. Cbh2 polypeptide. In another embodiment, the co-culture comprises at least one yeast host cell comprising a polynucleotide encoding a tethered Cochliobolus heterostrophus, Gibberella zeae, Irpex lacteus, Volvariella volvacea, or Piromyces sp. Cbh2 polypeptide. In one embodiment, all of the heterologous cellulases in the co-culture are secreted, and in another embodiment, all of the heterologous cellulases in the co-culture are tethered. In addition, other cellulases, such as externally added cellulases can be present in the co-culture.

According to the methods described herein, a host cell or group of host cells can comprise a vector or vectors which encode and express a combination of heterologous cellulases including a cellulase selected from the group consisting of Cochliobolus heterostrophus, Gibberella zeae, Irpex lacteus, Volvariella volvacea, and Piromyces sp. Cbh2. For example, a single host cell may express C. formosanus endoglucanase I, S. fibuligera β-glucosidase I, T. emersonii Cbh1, and a Cbh2 selected from the group consisting of Cochliobolus heterostrophus C4 cel7; Gibberella zeae K59 cel6; Irpex lacteus MC-2 cex3; Volvariella volvacea cbhII-I; and Piromyces sp. E2 cel6A. Alternatively, a group of cells could express a combination of cellulases, for example such that a first host cell expresses C. formosanus endoglucanase I, a second host cell expresses S. fibuligera β-glucosidase I, a third host cell expresses T. emersonii Cbh1, and a fourth host cell expresses a Cbh2 selected from the group consisting of Cochliobolus heterostrophus C4 cel7; Gibberella zeae K59 cel6; Irpex lacteus MC-2 cex3; Volvariella volvacea cbhII-I; and Piromyces sp. E2 cel6A. Similarly, a first host cell can express both C. formosanus endoglucanase I and S. fibuligera β-glucosidase I and a second host cell can express both T. emersonii Cbh1, and a Cbh2 selected from the group consisting of Cochliobolus heterostrophus C4 cel7; Gibberella zeae K59 cel6; Irpex lacteus MC-2 cex3; Volvariella volvacea cbhII-I; and Piromyces sp. E2 cel6A. In another embodiment, a single host cell or group of host cells may express T. reesi endoglucanase I, S. fibuligera β-glucosidase I, T. emersonii Cbh1, and a Cbh2 selected from the group consisting of Cochliobolus heterostrophus C4 cel7; Gibberella zeae K59 cel6; Irpex lacteus MC-2 cex3; Volvariella volvacea cbhII-I; and Piromyces sp. E2 cel6A.

### EXAMPLES

### Materials and Methods

### Media and Strain Cultivation

Escherichia coli strain DH5α (Invitrogen), or NEB 5 alpha (New England Biolabs) was used for plasmid transformation and propagation. Cells were grown in LB medium (5 g/L yeast extract, 5 g/L NaCl, 10 g/L tryptone) supplemented with ampicillin (100 mg/L), kanamycin (50 mg/L), or zeocin (20 mg/L). When zeocin selection was desired LB was adjusted to pH 7.0. Also, 15 g/L agar was added when solid media was desired.

Yeast strains were routinely grown in YPD (10 g/L yeast extract, 20 g/L peptone, 20 g/L glucose), YPC (10 g/L yeast extract, 20 g/L peptone, 20 g/L cellobiose), or YNB+glucose (6.7 g/L Yeast Nitrogen Base without amino acids, and supplemented with appropriate amino acids for strain, 20 g/L glucose) media with either G418 (250 mg/L unless specified) or zeocin (200 mg/L unless specified) for selection. 15 g/L agar was added for solid media.

### Molecular Methods

Standard protocols were followed for DNA manipulations (Sambrook et al. 1989). PCR was performed using Phusion polymerase (New England Biolabs) for cloning, and Taq polymerase (New England Biolabs) for screening transformants, and in some cases Advantage Polymerase (Clontech) for PCR of genes for correcting auxotrophies. Manufacturers guidelines were followed as supplied. Restriction enzymes were purchased from New Englad Biolabs and digests were set up according to the supplied guidelines. Ligations were performed using the Quick ligation kit (New England Biolabs) as specified by the manufacturer. Gel purification was performed using either Qiagen or Zymo research kits, PCR product and digest purifications were performed using Zymo research kits, and Qiagen midi and miniprep kits were used for purification of plasmid DNA.

### Yeast Transformation

A protocol for electrotransformation of yeast was developed based on Cho, K. M.; Yoo, Y. J. and Kang, H. S., Enzyme And Microbial Technology, 25: 23-30, (1999) and Ausubel, F. M., et al., Current Protocols in Molecular Biology. USA: John. Wiley and Sons, Inc. (1994). Linear fragments of DNA are created by restriction enzyme digestion utilizing unique restriction sites within the plasmid. The fragments are purified by precipitation with 3M sodium acetate and ice cold ethanol, subsequent washing with 70% ethanol, and resuspension in USB dH2O (DNAse and RNAse free, sterile water) after drying in a 70° C. vacuum oven.

Yeast cells, e.g., Saccharomyces cerevisiae, for transformation are prepared by growing to saturation in 5 mL YPD cultures. 4 mL of the culture is sampled, washed 2× with cold distilled water, and resuspended in 640 μL cold distilled water. 80 μL of 100 mM Tris-HCl, 10 mM EDTA, pH 7.5 (10×TE buffer—filter sterilized) and 80 μL of 1M lithium acetate, pH 7.5 (10× liAc—filter sterilized) is added and the cell suspension is incubated at 30° C. for 45 minutes with gentle shaking. 20 μL of 1M DTT is added and incubation continues for 15 minutes. The cells are then centrifuged, washed once with cold distilled water, and once with electroporation buffer (1M sorbitol, 20 mM HEPES), and finally resuspended in 267 μL electroporation buffer.

For electroporation, 10 μg of linearized DNA (measured by estimation on gel) is combined with 50 μL of the cell suspension in a sterile 1.5 mL microcentrifuge tube. The mixture is then transferred to a 0.2 cm electroporation cuvette, and a pulse of 1.4 kV (200Ω, 25 μF) is applied to the sample using, e.g., the Biorad Gene Pulser device. 1 mL of YPD with 1M sorbitol adjusted to pH 7.0 (YPDS) is placed in the cuvette and the cells are allowed to recover for ˜3 his. 100-200 μL cell suspension are spread out on YPDS agar plates with appropriate selection, which are incubated at 30° C. for 3-4 days until colonies appear.

### SDS-PAGE and Gel Staining

SDS-PAGE (sodium dodecyl sulfate polyacrylamide gel electrophoresis) was carried out as described by Laemmli (Nature 227: 680-685 (1970)) on a 10% gel at 100 V. A 20 μl sample of culture supernatant was mixed with SDS-PAGE loading buffer and incubated at 95° C. for 5 minutes before loading onto the gel. After protein separation, the gels were silver stained. Silver staining was performed by incubating the gels with shaking at room temperature in 1) 30% ethanol and 0.5% acetic acid (3×30 min); 2) 20% ethanol (10 min); 3) water (10 min); 4) sodium thiosulfate (0.2 g/L) (1 min); 5) water (2×20 seconds); 6) silver nitrate (2 g/L) (30 min); 7) water (5-10 seconds); 8) 37% formaldehyde (0.7 ml/L) and potassium carbonate (anhydr.) (30 g/L) and sodium thiosulfate (10 mg/L) (2×3 min or to desired intensity); 9) Tris base (50 g/L) and 2.5% acetic acid (1 min); and 10) water.

### Determination of Protein Concentration

To estimate specific activity of the Cbh2s the Bradford method (BioRad protein assay) was used as it is prescribed for use in microtiter plates, using the Gamma globulin standard. Before determination of protein concentration, supernatant samples were first subjected to the buffer exchange procedure as directed for the 2 mL Zeba desalt spin columns (Thermo Scientific).

### Measurement of Cellulase Activity

An Avicel conversion assay was used to measure the cellulolytic activity of yeast strains expressing CBHs. 2% Avicel cellulose in 50 mM Na-acetate, pH 5.0 is suspended and mixed well to make the suspension homogenous. The homogenous suspension is pipetted to the tubes (0.5 ml each). 0.5 ml of sample is added to each tube on the substrate. The samples can be: enzyme in buffer, yeast culture filtrate, inactivated yeast culture filtrate (to detect the background sugars from cultivation media) or buffer for blank. The tubes are incubated at 35° C. with shaking (1000 rpm). The samples (100 μl) are then removed after a pre-determined hydrolysis time, e.g., 0 h, 4 h, 24 h and 48 h, into separate tubes and spun down. 50 μl of supernatant is added to 100 μl of DNS reagent into a microplate. This mixture is then heated at 99° C. for 5 minutes. The absorbance is measured at 595 nm. The glucose equivalent formed (reducing sugars) is analyzed using DNS calibration by glucose standard.

The Dinitrosalicylic Acid Reagent Solution (DNS), 1% includes the following 3,5-dinitrosalicylic acid: 10 g; Sodium sulfite: 0.5 g; Sodium hydroxide: 10 g; water to 1 liter. The DNS is calibrated by glucose (using glucose samples with conc. 0, 1, 2, 3, 4, 5 and 6 g/l, the slope [S] is calculated, for DNS from May 8, 2007 S=0.0669). The DNS solution can be stored at 4° C. for several months.

### Example 1

### Cloning of Codon-Optimized Cbh2 Genes and their Expression in Saccharomyces cerevisiae

Cellobiohydrolase (cbh) genes from five fungal organisms (as indicated in Table 8 below) were selected for expression in yeast. The sequences were first codon-optimized for expression in Saccharomyces cerevisiae.

The software available at http://phenotype.biosci.umbc.edu/codon/sgd/index.php applying the CAI codon usage table suggested by Carbone et al. 2003 was utilized to generate an initial sequence that had a codon adaptation index (CAI) of 1.0, where three-letter sequences encoding for individual amino acid codons were replaced with those three-letter sequences known to be most frequently used in S. cerevisiae for the corresponding amino acid codons. The initial codon-optimized sequence generated by this software was then further modified. In particular, the software was utilized to identify certain stretches of sequence (e.g., sequences with 4, 5, 6, 7, 8, 9, or 10 contiguous A's or T's), and replace these sequences with three-letter sequences corresponding to the second most frequently utilized three-letter sequences in S. cerevisiae. In addition, for molecular cloning purposes, the website software was used to similarly replace certain restriction enzyme, including PacI, AscI, BamHI, BglII, EcoRI and XhoI. Finally other DNA software (DNAman) was used to check the DNA sequence for direct repeats, inverted repeats and mirror repeats with lengths of 10 bases or longer. These sequences were modified by manually replacing codons with “second best” codons. These steps resulted in a CAI of approximately 0.96 to 0.98. The codon-optimized sequences are shown in Table 5. The codon-optimized cbh2s listed in Table 5 above were cloned under control of the PGK1 promoter/terminator using pMU784 after excising the C. lucknowense cbh2b (Clcbh2b) gene with PacI and AscI enzymes. The sequence of pMU784 is

(SEQ ID NO: 21)
agctgtttcctgtgtgaaattgttatccgctcacaattccacacaacatacgagccggaagcataaagtgtaaagcctggggtgcct
aatgagtgaggtaactcacattaattgcgttgcgctcactgcccgctttccagtcgggaaacctgtcgtgccagctggattaatgaat
cggccaacgcgcggggagaggcggtttgcgtattgggcgctcttccgcttcctcgctcactgactcgctgcgctcggtcgttcgg
ctgcggcgagcggtatcagctcactcaaaggcggtaatacggttatccacagaatcaggggataacgcaggaaagaacatgtga
gcaaaaggccagcaaaaggccaggaaccgtaaaaaggccgcgttgctggcgtttttccataggctccgcccccctgacgagcat
cacaaaaatcgacgctcaagtcagaggtggcgaaacccgacaggactataaagataccaggcgtttccccctggaagctccctc
gtgcgctctcctgttccgaccctgccgcttaccggatacctgtccgcctttctcccttcgggaagcgtggcgctttctcaatgctcac
gctgtaggtatctcagttcggtgtaggtcgttcgctccaagctgggctgtgtgcacgaaccccccgttcagcccgaccgctgcgcc
ttatccggtaactatcgtcttgagtccaacccggtaagacacgacttatcgccactggcagcagccactggtaacaggattagcag
agcgaggtatgtaggcggtgctacagagttcttgaagtggtggcctaactacggctacactagaaggacagtatttggtatctgcg
ctctgctgaagccagttaccttcggaaaaagagttggtagctcttgatccggcaaacaaaccaccgctggtagcggtggtttttttgtt
tgcaagcagcagattacgcgcagaaaaaaaggatctcaagaagatcctttgatcttttctacggggtctgacgctcagtggaacga
aaactcacgttaagggattttggtcatgagattatcaaaaaggatcttcacctagatccttttaaattaaaaatgaagttttaaatcaatct
aaagtatatatgagtaaacttggtctgacagttaccaatgcttaatcagtgaggcacctatctcagcgatctgtctatttcgttcatccat
agttgcctgactccccgtcgtgtagataactacgatacgggagggcttaccatctggccccagtgctgcaatgataccgcgagacc
cacgctcaccggctccagatttatcagcaataaaccagccagccggaagggccgagcgcagaagtggtcctgcaactttatccg
cctccatccagtctattaattgttgccgggaagctagagtaagtagttcgccagttaatagtttgcgcaacgttgttgccattgctacag
gcatcgtggtgtcacgctcgtcgtttggtatggcttcattcagctccggttcccaacgatcaaggcgagttacatgatcccccatgttg
tgcaaaaaagcggttagctccttcggtcctccgatcgttgtcagaagtaagttggccgcagtgttatcactcatggttatggcagcac
tgcataattctcttactgtcatgccatccgtaagatgcttttctgtgactggtgagtactcaaccaagtcattctgagaatagtgtatgcg
gcgaccgagttgctcttgcccggcgtcaatacgggataataccgcgccacatagcagaactttaaaagtgctcatcattggaaaac
gttcttcggggcgaaaactctcaaggatcttaccgctgttgagatccagttcgatgtaacccactcgtgcacccaactgatcttcagc
atcttttactttcaccagcgtttctgggtgagcaaaaacaggaaggcaaaatgccgcaaaaaagggaataagggcgacacggaaa
tgttgaatactcatactcttcctttttcaatattattgaagcatttatcagggttattgtctcatgagcggatacatatttgaatgtatttagaa
aaataaacaaataggggttccgcgcacatttccccgaaaagtgccacctgacgtctaagaaaccattattatcatgacattaacctat
aaaaataggcgtatcacgaggccctttcgtctcgcgcgtttcggtgatgacggtgaaaacctctgacacatgcagctcccggagac
ggtcacagcttgtctgtaagcggatgccgggagcagacaagcccgtcagggcgcgtcagcgggtgttggcgggtgtcggggct
ggcttaactatgcggcatcagagcagattgtactgagagtgcaccataacgcatttaagcataaacacgcactatgccgttcttctca
tgtatatatatatacaggcaacacgcagatataggtgcgacgtgaacagtgagctgtatgtgcgcagctcgcgttgcattttcggaa
gcgctcgttttcggaaacgctttgaagttcctattccgaagttcctattctctagctagaaagtataggaacttcagagcgcttttgaaa
accaaaagcgctctgaagacgcactttcaaaaaaccaaaaacgcaccggactgtaacgagctactaaaatattgcgaataccgctt
ccacaaacattgctcaaaagtatctctttgctatatatctctgtgctatatccctatataacctacccatccacctttcgctccttgaacttg
catctaaactcgacctctacattttttatgtttatctctagtattactctttagacaaaaaaattgtagtaagaactattcatagagtgaatc
gaaaacaatacgaaaatgtaaacatttcctatacgtagtatatagagacaaaatagaagaaaccgttcataattttctgaccaatgaa
gaatcatcaacgctatcactttctgttcacaaagtatgcgcaatccacatcggtatagaatataatcggggatgcctttatcttgaaaaa
atgcacccgcagcttcgctagtaatcagtaaacgcgggaagtggagtcaggctttttttatggaagagaaaatagacaccaaagta
gccttcttctaaccttaacggacctacagtgcaaaaagttatcaagagactgcattatagagcgcacaaaggagaaaaaaagtaat
ctaagatgctttgttagaaaaatagcgctctcgggatgcatttttgtagaacaaaaaagaagtatagattctttgttggtaaaatagcgc
tctcgcgttgcatttctgttctgtaaaaatgcagctcagattctttgtttgaaaaattagcgctctcgcgttgcatttttgttttacaaaaatg
aagcacagattcttcgttggtaaaatagcgctttcgcgttgcatttctgttctgtaaaaatgcagctcagattctttgtttgaaaaattagc
gctctcgcgttgcatttttgttctacaaaatgaagcacagatgcttcgttaacaaagatatgctattgaagtgcaagatggaaacgcag
aaaatgaaccggggatgcgacgtgcaagattacctatgcaatagatgcaatagtttctccaggaaccgaaatacatacattgtcttc
cgtaaagcgctagactatatattattatacaggttcaaatatactatctgtttcagggaaaactcccaggttcggatgttcaaaattcaat
gatgggtaacaagagcttttcaattcatcattttttttttattcttttttttgatttcggtttctttgaaatttttttgattcggtaatctccgaacag
aaggaagaacgaaggaaggagcacagacttagattggtatatatacgcatatgtagtgttgaagaaacatgaaattgcccagtatt
cttaacccaactgcacagaacaaaaaccgaaacgaagataaatcatgtcgaaagctacatataaggaacgtgctgctactcatcct
agtcctgttgctgccaagctatttaatatcatgcacgaaaagcaaacaaacttgtgtgcttcattggatgttcgtaccaccaaggaatt
actggagttagttgaagcattaggtcccaaaatttgtttactaaaaacacatgtggatatcttgactgatttttccatggagggcacagt
taagccgctaaaggcattatccgccaagtacaattttttactcttcgaagacagaaaatttgctgacattggtaatacagtcaaattgca
gtactctgcgggtgtatacagaatagcagaatgggcagacattacgaatgcacacggtgtggtgggcccaggtattgttagcggtt
tgaagcaggcggcagaagaagtaacaaaggaacctagaggccttttgatgttagcagaattgtcatgcaagggctccctatctact
ggagaatatactaagggtactgttgacattgcgaagagcgacaaagattttgttatcggctttattgctcaaagagacatgggtggaa
gagatgaaggttacgattggttgattatgacacccggtgtgggtttagatgacaagggagacgcattgggtcaacagtatagaacc
gtggatgatgtggtctctacaggatctgacattattattgttggaagaggactatttgcaaagggaagggatgctaaggtagagggt
gaacgttacagaaaagcaggctgggaagcatatttgagaagatgcggccagcaaaactaaaaaactgtattataagtaaatgcat
gtatactaaactcacaaattagagcttcaatttaattatatcagttattaccctatgcggtgtgaaataccgcacagatgcgtaaggag
aaaataccgcatcaggcgccattcgccattcaggctgcgcaactgttgggaagggcgatcggtgcgggcctcttcgctattacgc
cagctggcgaaggggggatgtgctgcaaggcgattaagttgggtaacgccagggttttcccagtcacgacgttgtaaaacgacg
gccagtgccaagctttctaactgatctatccaaaactgaaaattacattcttgattaggtttatcacaggcaaatgtaatttgtggtatttt
gccgttcaaaatctgtagaattttctcattggtcacattacaacctgaaaatactttatctacaatcataccattcttataacatgtcccctt
aatactaggatcaggcatgaacgcatcacagacaaaatcttcttgacaaacgtcacaattgatccctccccatccgttatcacaatga
caggtgtcattttgtgctcttatgggacgatccttattaccgctttcatccggtgatagaccgccacagaggggcagagagcaatcat
cacctgcaaacccttctatacactcacatctaccagtgtacgaattgcattcagaaaactgtttgcattcaaaaataggtagcatacaa
ttaaaacatggcgggcacgtatcattgcccttatcttgtgcagttagacgcgaatttttcgaagaagtaccttcaaagaatggggtctc
atcttgttttgcaagtaccactgagcaggataataatagaaatgataatatactatagtagagataacgtcgatgacttcccatactgta
attgcttttagttgtgtatttttagtgtgcaagtttctgtaaatcgattaatttttttttctttcctctttttattaaccttaatttttattttagattcct
gacttcaactcaagacgcacagatattataacatctgcacaataggcatttgcaagaattactcgtgagtaaggaaagagtgagga
actatcgcatacctgcatttaaagatgccgatttgggcgcgaatcctttattttggcttcaccctcatactattatcagggccagaaaaa
ggaagtgtttccctccttcttgaattgatgttaccctcataaagcacgtggcctcttatcgagaaagaaattaccgtcgctcgtgatttgt
ttgcaaaaagaacaaaactgaaaaaacccagacacgctcgacttcctgtcttcctattgattgcagcttccaatttcgtcacacaaca
aggtcctagcgacggctcacaggttttgtaacaagcaatcgaaggttctggaatggcgggaaagggtttagtaccacatgctatga
tgcccactgtgatctccagagcaaagttcgttcgatcgtactgttactctctctctttcaaacagaattgtccgaatcgtgtgacaacaa
cagcctgttctcacacactcttttcttctaaccaagggggtggtttagtttagtagaacctcgtgaaacttacatttacatatatataaact
tgcataaattggtcaatgcaagaaatacatatttggtcttttctaattcgtagtttttcaagttcttagatgctttctttttctcttttttacagat
catcaaggaagtaattatctactttttacaacaaatataaaacttaattaaacaatggccaagaagttgttcattaccgctgccttagctg
ccgcagtgcttgctgcaccagtgatcgaagagagacaaaattgcggagccgtctggacacagtgcggaggcaacggctggcaa
ggcccaacatgttgtgcttctggctcaacgtgcgtggcacagaacgagtggtattcccagtgccttccaaactcccaggtgacttctt
caacaacccccagctcaacgtctacttcacagagatccacaagtacctcttctagcacaaccagaagtggctcatcctcatctagca
gtacgacccctccacccgtatcaagtcctgtcacgagtatccctggcggagcaacctcaacagccagttattccggcaatcctttct
ctggagtgagattatttgcaaacgactattatagatcagaggttcacaaccttgcaattccttctatgacgggaaccctagccgcaaa
ggcttccgccgtagcagaagtccctagtttccaatggcttgacagaaacgttacaatagatacacttatggtacagactttatctcag
gttagagctttgaataaggccggtgccaacccaccttatgctgcccaattagtagtctatgacttgccagatagagactgtgctgccg
cagcttctaatggtgaattttccatcgcaaatggcggagctgcaaactatagatcatacattgatgcaataagaaaacacatcattga
gtattctgatattagaataatccttgtgattgaaccagactccatggctaatatggttaccaacatgaatgtagccaagtgttctaacgc
agcttccacataccatgagctaaccgtatatgcattaaaacaactgaatctacctaacgttgctatgtacttagatgccggtcatgccg
gatggttgggctggcctgcaaatatccaacccgcagctgaattgttcgctggaatctacaacgacgccggaaagcccgctgccgt
tagaggcttagccacaaatgttgcaaattacaacgcttggtcaattgctagtgccccttcttatacctcaccaaatcctaactacgatg
agaaacattacatagaagcattttccccattgttaaactccgctggattccctgccagattcatcgtggataccggtagaaacggcaa
acaaccaactggacaacaacaatggggagattggtgtaacgtcaagggaaccggcttcggcgtcaggcctacggcaaacaccg
gacacgagctagtcgacgcttttgtatgggttaagccaggtggcgaaagtgacggaacaagtgacacgagtgctgcaagatacg
attaccactgtggtctgtccgacgctttacagcccgcccccgaggctggacaatggttccaggcttattttgaacaattgttaacgaa
cgcaaatccaccattctaaggcgcgccgaattcgagagactcgagactgaatcggatcgatcccgggcccgtcgagggatctgc
gatagatcaatttttttcttttctctttccccatcctttacgctaaaataatagtttattttattttttgaatattttttatttatatacgtatatataga
ctattatttatcttttaatgattattaagatttttattaaaaaaaaattcgctcctcttttaatgcctttatgcagtttttttttcccattcgatatttc
tatgttcgggttcagcgtattttaagtttaataactcgaaaattctgcgttcgttaaagcttgcatgcctgcaggtcgactctagaggatc
cccgggtaccgagctcgaattaattcgtaatcatggtcat.

The resulting plasmids are summarized below in Tables 8 and 9.

TABLE 8
Cbh2 plasmid descriptions.

| | | Theoretical |
|---|---|---|
| | Expression | enzyme size |
| Organism & Gene: | plasmid: | Da* |
| Cochliobolus heterostrophus C4 cel7 | pRDH150 | 41647.29 |
| Gibberella zeae K59 cel6 | pRDH151 | 49032.64 |
| Irpex lacteus MC-2 cex3 | pRDH152 | 47388.73 |
| Volvariella volvacea cbhII-I | pRDH153 | 46981.60 |
| Piromyces sp. E2 cel6A | pRDH154 | 53914.95 |

TABLE 9
Cbh2 plasmid sequences.

pRDH150
agctgtttcctgtgtgaaattgttatccgctcacaattccacacaacatacgagccggaagcataaagtgtaaagcctggggtgcctaatgagt
gaggtaactcacattaattgcgttgcgctcactgcccgctttccagtcgggaaacctgtcgtgccagctggattaatgaatcggccaacgcgc
ggggagaggcggtttgcgtattgggcgctcttccgcttcctcgctcactgactcgctgcgctcggtcgttcggctgcggcgagcggtatcag
ctcactcaaaggcggtaatacggttatccacagaatcaggggataacgcaggaaagaacatgtgagcaaaaggccagcaaaaggccagg
aaccgtaaaaaggccgcgttgctggcgtttttccataggctccgcccccctgacgagcatcacaaaaatcgacgctcaagtcagaggtggc
gaaacccgacaggactataaagataccaggcgtttccccctggaagctccctcgtgcgctctcctgttccgaccctgccgcttaccggatac
ctgtccgcctttctcccttcgggaagcgtggcgctttctcaatgctcacgctgtaggtatctcagttcggtgtaggtcgttcgctccaagctggg
ctgtgtgcacgaaccccccgttcagcccgaccgctgcgccttatccggtaactatcgtcttgagtccaacccggtaagacacgacttatcgcc
actggcagcagccactggtaacaggattagcagagcgaggtatgtaggcggtgctacagagttcttgaagtggtggcctaactacggctac
actagaaggacagtatttggtatctgcgctctgctgaagccagttaccttcggaaaaagagttggtagctcttgatccggcaaacaaaccacc
gctggtagcggtggtttttttgtttgcaagcagcagattacgcgcagaaaaaaaggatctcaagaagatcctttgatcttttctacggggtctga
cgctcagtggaacgaaaactcacgttaagggattttggtcatgagattatcaaaaaggatcttcacctagatccttttaaattaaaaatgaagtttt
aaatcaatctaaagtatatatgagtaaacttggtctgacagttaccaatgcttaatcagtgaggcacctatctcagcgatctgtctatttcgttcatc
catagttgcctgactccccgtcgtgtagataactacgatacgggagggcttaccatctggccccagtgctgcaatgataccgcgagacccac
gctcaccggctccagatttatcagcaataaaccagccagccggaagggccgagcgcagaagtggtcctgcaactttatccgcctccatcca
gtctattaattgttgccgggaagctagagtaagtagttcgccagttaatagtttgcgcaacgttgttgccattgctacaggcatcgtggtgtcac
gctcgtcgtttggtatggcttcattcagctccggttcccaacgatcaaggcgagttacatgatcccccatgttgtgcaaaaaagcggttagctc
cttcggtcctccgatcgttgtcagaagtaagttggccgcagtgttatcactcatggttatggcagcactgcataattctcttactgtcatgccatcc
gtaagatgcttttctgtgactggtgagtactcaaccaagtcattctgagaatagtgtatgcggcgaccgagttgctcttgcccggcgtcaatac
gggataataccgcgccacatagcagaactttaaaagtgctcatcattggaaaacgttcttcggggcgaaaactctcaaggatcttaccgctgtt
gagatccagttcgatgtaacccactcgtgcacccaactgatcttcagcatcttttactttcaccagcgtttctgggtgagcaaaaacaggaagg
caaaatgccgcaaaaaagggaataagggcgacacggaaatgttgaatactcatactcttcctttttcaatattattgaagcatttatcagggttat
tgtctcatgagcggatacatatttgaatgtatttagaaaaataaacaaataggggttccgcgcacatttccccgaaaagtgccacctgacgtcta
agaaaccattattatcatgacattaacctataaaaataggcgtatcacgaggccctttcgtctcgcgcgtttcggtgatgacggtgaaaacctct
gacacatgcagctcccggagacggtcacagcttgtctgtaagcggatgccgggagcagacaagcccgtcagggcgcgtcagcgggtgtt
ggcgggtgtcggggctggcttaactatgcggcatcagagcagattgtactgagagtgcaccataacgcatttaagcataaacacgcactatg
ccgttcttctcatgtatatatatatacaggcaacacgcagatataggtgcgacgtgaacagtgagctgtatgtgcgcagctcgcgttgcattttc
ggaagcgctcgttttcggaaacgctttgaagttcctattccgaagttcctattctctagctagaaagtataggaacttcagagcgcttttgaaaac
caaaagcgctctgaagacgcactttcaaaaaaccaaaaacgcaccggactgtaacgagctactaaaatattgcgaataccgcttccacaaa
cattgctcaaaagtatctctttgctatatatctctgtgctatatccctatataacctacccatccacctttcgctccttgaacttgcatctaaactcgac
ctctacattttttatgtttatctctagtattactctttagacaaaaaaattgtagtaagaactattcatagagtgaatcgaaaacaatacgaaaatgta
aacatttcctatacgtagtatatagagacaaaatagaagaaaccgttcataattttctgaccaatgaagaatcatcaacgctatcactttctgttca
caaagtatgcgcaatccacatcggtatagaatataatcggggatgcctttatcttgaaaaaatgcacccgcagcttcgctagtaatcagtaaac
gcgggaagtggagtcaggctttttttatggaagagaaaatagacaccaaagtagccttcttctaaccttaacggacctacagtgcaaaaagtt
atcaagagactgcattatagagcgcacaaaggagaaaaaaagtaatctaagatgctttgttagaaaaatagcgctctcgggatgcatttttgta
gaacaaaaaagaagtatagattctttgttggtaaaatagcgctctcgcgttgcatttctgttctgtaaaaatgcagctcagattctttgtttgaaaaa
ttagcgctctcgcgttgcatttttgttttacaaaaatgaagcacagattcttcgttggtaaaatagcgctttcgcgttgcatttctgttctgtaaaaat
gcagctcagattctttgtttgaaaaattagcgctctcgcgttgcatttttgttctacaaaatgaagcacagatgcttcgttaacaaagatatgctatt
gaagtgcaagatggaaacgcagaaaatgaaccggggatgcgacgtgcaagattacctatgcaatagatgcaatagtttctccaggaaccg
aaatacatacattgtcttccgtaaagcgctagactatatattattatacaggttcaaatatactatctgtttcagggaaaactcccaggttcggatgt
tcaaaattcaatgatgggtaacaagagcttttcaattcatcattttttttttattcttttttttgatttcggtttctttgaaatttttttgattcggtaa
tctccgaacagaaggaagaacgaaggaaggagcacagacttagattggtatatatacgcatatgtagtgttgaagaaacatgaaattgcccagtattct
taacccaactgcacagaacaaaaaccgaaacgaagataaatcatgtcgaaagctacatataaggaacgtgctgctactcatcctagtcctgtt
gctgccaagctatttaatatcatgcacgaaaagcaaacaaacttgtgtgcttcattggatgttcgtaccaccaaggaattactggagttagttga
agcattaggtcccaaaatttgtttactaaaaacacatgtggatatcttgactgatttttccatggagggcacagttaagccgctaaaggcattatc
cgccaagtacaattttttactcttcgaagacagaaaatttgctgacattggtaatacagtcaaattgcagtactctgcgggtgtatacagaatagc
agaatgggcagacattacgaatgcacacggtgtggtgggcccaggtattgttagcggtttgaagcaggcggcagaagaagtaacaaagga
acctagaggccttttgatgttagcagaattgtcatgcaagggctccctatctactggagaatatactaagggtactgttgacattgcgaagagc
gacaaagattttgttatcggctttattgctcaaagagacatgggtggaagagatgaaggttacgattggttgattatgacacccggtgtgggttt
agatgacaagggagacgcattgggtcaacagtatagaaccgtggatgatgtggtctctacaggatctgacattattattgttggaagaggact
atttgcaaagggaagggatgctaaggtagagggtgaacgttacagaaaagcaggctgggaagcatatttgagaagatgcggccagcaaa
actaaaaaactgtattataagtaaatgcatgtatactaaactcacaaattagagcttcaatttaattatatcagttattaccctatgcggtgtgaaat
accgcacagatgcgtaaggagaaaataccgcatcaggcgccattcgccattcaggctgcgcaactgttgggaagggcgatcggtgcggg
cctcttcgctattacgccagctggcgaaggggggatgtgctgcaaggcgattaagttgggtaacgccagggttttcccagtcacgacgttgt
aaaacgacggccagtgccaagctttctaactgatctatccaaaactgaaaattacattcttgattaggtttatcacaggcaaatgtaatttgtggt
attttgccgttcaaaatctgtagaattttctcattggtcacattacaacctgaaaatactttatctacaatcataccattcttataacatgtccccttaa
tactaggatcaggcatgaacgcatcacagacaaaatcttcttgacaaacgtcacaattgatccctccccatccgttatcacaatgacaggtgtca
ttttgtgctcttatgggacgatccttattaccgctttcatccggtgatagaccgccacagaggggcagagagcaatcatcacctgcaaaccctt
ctatacactcacatctaccagtgtacgaattgcattcagaaaactgtttgcattcaaaaataggtagcatacaattaaaacatggcgggcacgta
tcattgcccttatcttgtgcagttagacgcgaatttttcgaagaagtaccttcaaagaatggggtctcatcttgttttgcaagtaccactgagcag
gataataatagaaatgataatatactatagtagagataacgtcgatgacttcccatactgtaattgcttttagttgtgtatttttagtgtgcaagtttct
gtaaatcgattaatttttttttctttcctctttttattaaccttaatttttattttagattcctgacttcaactcaagacgcacagatattataacatct
gcacaataggcatttgcaagaattactcgtgagtaaggaaagagtgaggaactatcgcatacctgcatttaaagatgccgatttgggcgcgaatcctt
tattttggcttcaccctcatactattatcagggccagaaaaaggaagtgtttccctccttcttgaattgatgttaccctcataaagcacgtggcctc
ttatcgagaaagaaattaccgtcgctcgtgatttgtttgcaaaaagaacaaaactgaaaaaacccagacacgctcgacttcctgtcttcctattg
attgcagcttccaatttcgtcacacaacaaggtcctagcgacggctcacaggttttgtaacaagcaatcgaaggttctggaatggcgggaaa
gggtttagtaccacatgctatgatgcccactgtgatctccagagcaaagttcgttcgatcgtactgttactctctctctttcaaacagaattgtccg
aatcgtgtgacaacaacagcctgttctcacacactcttttcttctaaccaagggggtggtttagtttagtagaacctcgtgaaacttacatttacat
atatataaacttgcataaattggtcaatgcaagaaatacatatttggtcttttctaattcgtagtttttcaagttcttagatgctttctttttctctttt
ttacagatcatcaaggaagtaattatctactttttacaacaaatataaaacttaattaaAATGTTGTCTAACGTTTTTTTGACTG
CTGCTTTGGCTGCTGGTTTGGCTCAAGCTTTGCCACAAGCTACTCCAACTCCAACTGC
TGCTCCATCTGGTAATCCATTTGCTGGTAAGAATTTTTACGCTAACCCATATTATTCT
TCAGAAGTTCATACTTTGGCTATGCCATCTTTGCCAGCTTCATTGAAACCAGCTGCTA
CTGCTGTTGCTAAAGTTGGTTCTTTTGTTTGGATGGATACTATGGCTAAAGTTCCATT
GATGGATACTTACTTGGCTGATATTAAAGCTAAAAATGCTGCTGGTGCTAATTTGAT
GGGTACTTTCGTTGTTTATGATTTGCCAGATAGAGATTGTGCTGCTTTAGCTTCTAAT
GGTGAATTGAAAATTGATGAAGGTGGTGTTGAAAAATACAAGACACAATACATTGA
TAAGATTGCTGCTATTATCAAAAAGTACCCAGATGTTAAGATTAATTTGGCTATTGA
ACCAGATTCTTTGGCTAATATGGTTACTAATATGGGTGTTCAAAAATGTTCTAGAGCT
GCTCCATATTACAAAGAATTGACTGCTTATGCTTTGAAAACTTTGAACTTCAACAAC
GTTGACATGTATATGGATGGTGGTCATGCTGGTTGGTTGGGTTGGGATGCTAATATT
GGTCCAACTGCTAAATTGTTTGCTGAAGTTTACAAAGCTGCTGGTTCTCCAAGAGGT
GTTAGAGGTATTGTTACAAACGTTTCTAATTACAACGCTTTGAGAGTTTCTTCTTGTC
CATCTATTACTCAAGGTAACAAGAATTGTGATGAAGAAAGATACATTAATGCTTTGG
CTCCATTGTTGAAAAATGAAGGTTTTCCAGCTCATTTTATTGTTGATCAAGGTAGATC
AGGTAAAGTTCCAACTAATCAACAAGAATGGGGTGATTGGTGTAATGTTTCTGGTGC
TGGTTTTGGTACTAGACCAACTACTAATACTGGTAATGCTTTGATTGATGCTATTGTT
TGGGTTAAACCAGGTGGTGAATCTGATGGTACTTCTGATACTTCTGCTGCAAGATAT
GATGCTCATTGTGGTAGAAATTCTGCTTTTAAACCAGCTCCAGAAGCTGGTACTTGG
TTTCAAGCTTACTTTGAAATGTTGTTGAAGAATGCTAATCCAGCTTTGGCATTATAAg
gcgcgccgaattcgagagactcgagactgaatcggatcgatcccgggcccgtcgagggatctgcgatagatcaatttttttcttttctctttccc
catcctttacgctaaaataatagtttattttattttttgaatattttttatttatatacgtatatatagactattatttatcttttaatgattattaaga
tttttattaaaaaaaaattcgctcctcttttaatgcctttatgcagtttttttttcccattcgatatttctatgttcgggttcagcgtattttaagttta
ataactcgaaaattctgcgttcgttaaagcttgcatgcctgcaggtcgactctagaggatccccgggtaccgagctcgaattaattcgtaatcatggtca
t
(SEQ ID NO: 37)
pRDH151
agctgtttcctgtgtgaaattgttatccgctcacaattccacacaacatacgagccggaagcataaagtgtaaagcctggggtgcctaatgagt
gaggtaactcacattaattgcgttgcgctcactgcccgctttccagtcgggaaacctgtcgtgccagctggattaatgaatcggccaacgcgc
ggggagaggcggtttgcgtattgggcgctcttccgcttcctcgctcactgactcgctgcgctcggtcgttcggctgcggcgagcggtatcag
ctcactcaaaggcggtaatacggttatccacagaatcaggggataacgcaggaaagaacatgtgagcaaaaggccagcaaaaggccagg
aaccgtaaaaaggccgcgttgctggcgtttttccataggctccgcccccctgacgagcatcacaaaaatcgacgctcaagtcagaggtggc
gaaacccgacaggactataaagataccaggcgtttccccctggaagctccctcgtgcgctctcctgttccgaccctgccgcttaccggatac
ctgtccgcctttctcccttcgggaagcgtggcgctttctcaatgctcacgctgtaggtatctcagttcggtgtaggtcgttcgctccaagctggg
ctgtgtgcacgaaccccccgttcagcccgaccgctgcgccttatccggtaactatcgtcttgagtccaacccggtaagacacgacttatcgcc
actggcagcagccactggtaacaggattagcagagcgaggtatgtaggcggtgctacagagttcttgaagtggtggcctaactacggctac
actagaaggacagtatttggtatctgcgctctgctgaagccagttaccttcggaaaaagagttggtagctcttgatccggcaaacaaaccacc
gctggtagcggtggtttttttgtttgcaagcagcagattacgcgcagaaaaaaaggatctcaagaagatcctttgatcttttctacggggtctga
cgctcagtggaacgaaaactcacgttaagggattttggtcatgagattatcaaaaaggatcttcacctagatccttttaaattaaaaatgaagtttt
aaatcaatctaaagtatatatgagtaaacttggtctgacagttaccaatgcttaatcagtgaggcacctatctcagcgatctgtctatttcgttcatc
catagttgcctgactccccgtcgtgtagataactacgatacgggagggcttaccatctggccccagtgctgcaatgataccgcgagacccac
gctcaccggctccagatttatcagcaataaaccagccagccggaagggccgagcgcagaagtggtcctgcaactttatccgcctccatcca
gtctattaattgttgccgggaagctagagtaagtagttcgccagttaatagtttgcgcaacgttgttgccattgctacaggcatcgtggtgtcac
gctcgtcgtttggtatggcttcattcagctccggttcccaacgatcaaggcgagttacatgatcccccatgttgtgcaaaaaagcggttagctc
cttcggtcctccgatcgttgtcagaagtaagttggccgcagtgttatcactcatggttatggcagcactgcataattctcttactgtcatgccatcc
gtaagatgcttttctgtgactggtgagtactcaaccaagtcattctgagaatagtgtatgcggcgaccgagttgctcttgcccggcgtcaatac
gggataataccgcgccacatagcagaactttaaaagtgctcatcattggaaaacgttcttcggggcgaaaactctcaaggatcttaccgctgtt
gagatccagttcgatgtaacccactcgtgcacccaactgatcttcagcatcttttactttcaccagcgtttctgggtgagcaaaaacaggaagg
caaaatgccgcaaaaaagggaataagggcgacacggaaatgttgaatactcatactcttcctttttcaatattattgaagcatttatcagggttat
tgtctcatgagcggatacatatttgaatgtatttagaaaaataaacaaataggggttccgcgcacatttccccgaaaagtgccacctgacgtcta
agaaaccattattatcatgacattaacctataaaaataggcgtatcacgaggccctttcgtctcgcgcgtttcggtgatgacggtgaaaacctct
gacacatgcagctcccggagacggtcacagcttgtctgtaagcggatgccgggagcagacaagcccgtcagggcgcgtcagcgggtgtt
ggcgggtgtcggggctggcttaactatgcggcatcagagcagattgtactgagagtgcaccataacgcatttaagcataaacacgcactatg
ccgttcttctcatgtatatatatatacaggcaacacgcagatataggtgcgacgtgaacagtgagctgtatgtgcgcagctcgcgttgcattttc
ggaagcgctcgttttcggaaacgctttgaagttcctattccgaagttcctattctctagctagaaagtataggaacttcagagcgcttttgaaaac
caaaagcgctctgaagacgcactttcaaaaaaccaaaaacgcaccggactgtaacgagctactaaaatattgcgaataccgcttccacaaa
cattgctcaaaagtatctctttgctatatatctctgtgctatatccctatataacctacccatccacctttcgctccttgaacttgcatctaaactcgac
ctctacattttttatgtttatctctagtattactctttagacaaaaaaattgtagtaagaactattcatagagtgaatcgaaaacaatacgaaaatgta
aacatttcctatacgtagtatatagagacaaaatagaagaaaccgttcataattttctgaccaatgaagaatcatcaacgctatcactttctgttca
caaagtatgcgcaatccacatcggtatagaatataatcggggatgcctttatcttgaaaaaatgcacccgcagcttcgctagtaatcagtaaac
gcgggaagtggagtcaggctttttttatggaagagaaaatagacaccaaagtagccttcttctaaccttaacggacctacagtgcaaaaagtt
atcaagagactgcattatagagcgcacaaaggagaaaaaaagtaatctaagatgctttgttagaaaaatagcgctctcgggatgcatttttgta
gaacaaaaaagaagtatagattctttgttggtaaaatagcgctctcgcgttgcatttctgttctgtaaaaatgcagctcagattctttgtttgaaaaa
ttagcgctctcgcgttgcatttttgttttacaaaaatgaagcacagattcttcgttggtaaaatagcgctttcgcgttgcatttctgttctgtaaaaat
gcagctcagattctttgtttgaaaaattagcgctctcgcgttgcatttttgttctacaaaatgaagcacagatgcttcgttaacaaagatatgctatt
gaagtgcaagatggaaacgcagaaaatgaaccggggatgcgacgtgcaagattacctatgcaatagatgcaatagtttctccaggaaccg
aaatacatacattgtcttccgtaaagcgctagactatatattattatacaggttcaaatatactatctgtttcagggaaaactcccaggttcggatgt
tcaaaattcaatgatgggtaacaagagcttttcaattcatcattttttttttattcttttttttgatttcggtttctttgaaatttttttgattcggtaa
tctccgaacagaaggaagaacgaaggaaggagcacagacttagattggtatatatacgcatatgtagtgttgaagaaacatgaaattgcccagtattct
taacccaactgcacagaacaaaaaccgaaacgaagataaatcatgtcgaaagctacatataaggaacgtgctgctactcatcctagtcctgtt
gctgccaagctatttaatatcatgcacgaaaagcaaacaaacttgtgtgcttcattggatgttcgtaccaccaaggaattactggagttagttga
agcattaggtcccaaaatttgtttactaaaaacacatgtggatatcttgactgatttttccatggagggcacagttaagccgctaaaggcattatc
cgccaagtacaattttttactcttcgaagacagaaaatttgctgacattggtaatacagtcaaattgcagtactctgcgggtgtatacagaatagc
agaatgggcagacattacgaatgcacacggtgtggtgggcccaggtattgttagcggtttgaagcaggcggcagaagaagtaacaaagga
acctagaggccttttgatgttagcagaattgtcatgcaagggctccctatctactggagaatatactaagggtactgttgacattgcgaagagc
gacaaagattttgttatcggctttattgctcaaagagacatgggtggaagagatgaaggttacgattggttgattatgacacccggtgtgggttt
agatgacaagggagacgcattgggtcaacagtatagaaccgtggatgatgtggtctctacaggatctgacattattattgttggaagaggact
atttgcaaagggaagggatgctaaggtagagggtgaacgttacagaaaagcaggctgggaagcatatttgagaagatgcggccagcaaa
actaaaaaactgtattataagtaaatgcatgtatactaaactcacaaattagagcttcaatttaattatatcagttattaccctatgcggtgtgaaat
accgcacagatgcgtaaggagaaaataccgcatcaggcgccattcgccattcaggctgcgcaactgttgggaagggcgatcggtgcggg
cctcttcgctattacgccagctggcgaaggggggatgtgctgcaaggcgattaagttgggtaacgccagggttttcccagtcacgacgttgt
aaaacgacggccagtgccaagctttctaactgatctatccaaaactgaaaattacattcttgattaggtttatcacaggcaaatgtaatttgtggt
attttgccgttcaaaatctgtagaattttctcattggtcacattacaacctgaaaatactttatctacaatcataccattcttataacatgtccccttaa
tactaggatcaggcatgaacgcatcacagacaaaatcttcttgacaaacgtcacaattgatccctccccatccgttatcacaatgacaggtgtca
ttttgtgctcttatgggacgatccttattaccgctttcatccggtgatagaccgccacagaggggcagagagcaatcatcacctgcaaaccctt
ctatacactcacatctaccagtgtacgaattgcattcagaaaactgtttgcattcaaaaataggtagcatacaattaaaacatggcgggcacgta
tcattgcccttatcttgtgcagttagacgcgaatttttcgaagaagtaccttcaaagaatggggtctcatcttgttttgcaagtaccactgagcag
gataataatagaaatgataatatactatagtagagataacgtcgatgacttcccatactgtaattgcttttagttgtgtatttttagtgtgcaagtttct
gtaaatcgattaatttttttttctttcctctttttattaaccttaatttttattttagattcctgacttcaactcaagacgcacagatattataacatct
gcacaataggcatttgcaagaattactcgtgagtaaggaaagagtgaggaactatcgcatacctgcatttaaagatgccgatttgggcgcgaatcctt
tattttggcttcaccctcatactattatcagggccagaaaaaggaagtgtttccctccttcttgaattgatgttaccctcataaagcacgtggcctc
ttatcgagaaagaaattaccgtcgctcgtgatttgtttgcaaaaagaacaaaactgaaaaaacccagacacgctcgacttcctgtcttcctattg
attgcagcttccaatttcgtcacacaacaaggtcctagcgacggctcacaggttttgtaacaagcaatcgaaggttctggaatggcgggaaa
gggtttagtaccacatgctatgatgcccactgtgatctccagagcaaagttcgttcgatcgtactgttactctctctctttcaaacagaattgtccg
aatcgtgtgacaacaacagcctgttctcacacactcttttcttctaaccaagggggtggtttagtttagtagaacctcgtgaaacttacatttacat
atatataaacttgcataaattggtcaatgcaagaaatacatatttggtcttttctaattcgtagtttttcaagttcttagatgctttctttttctctttt
ttacagatcatcaaggaagtaattatctactttttacaacaaatataaaacttaattaaAATGACTGCTTACAAATTGTTTTTGG
CTGCTGCTTTTGCTGCTACTGCTTTGGCTGCTCCAGTTGAAGAAAGACAATCTTGTTC
TAATGGTGTTTGGTCACAATGTGGTGGTCAAAATTGGTCTGGTACTCCATGTTGTACA
TCTGGTAACAAGTGTGTTAAGGTTAATGATTTCTACTCTCAATGTCAACCAGGTTCTG
CTGATCCATCTCCAACTTCTACTATTGTTTCTGCTACTACTACTAAAGCTACTACTAC
AGGTTCTGGTGGTTCTGTTACTTCTCCACCACCAGTTGCTACAAACAATCCATTTTCT
GGTGTTGATTTGTGGGCAAACAATTATTACAGATCAGAAGTTTCTACTTTGGCTATTC
CAAAATTGTCTGGTGCTATGGCTACTGCTGCTGCAAAAGTTGCTGATGTTCCATCTTT
TCAATGGATGGATACTTACGATCATATTTCTTTCATGGAAGATTCTTTGGCTGATATT
AGAAAAGCAAACAAAGCAGGTGGTAATTATGCTGGTCAATTCGTTGTTTATGATTTG
CCAGATAGAGATTGTGCTGCTGCTGCTTCTAATGGTGAATACTCTTTGGATAAAGAT
GGTAAAAACAAGTACAAAGCTTATATTGCTGATCAAGGTATTTTGCAAGATTACTCT
GATACTAGAATCATTTTGGTTATTGAACCAGATTCTTTAGCTAACATGGTTACTAATA
TGAATGTTCCAAAATGTGCTAATGCTGCTTCTGCTTACAAAGAATTGACTATTCATGC
TTTGAAAGAATTGAATTTGCCAAACGTTTCAATGTATATTGATGCTGGTCATGGTGGT
TGGTTGGGTTGGCCAGCTAATTTGCCACCTGCTGCTCAATTGTATGGTCAATTGTACA
AAGATGCTGGTAAACCATCTAGATTGAGAGGTTTGGTTACTAATGTTTCTAATTACA
ACGCTTGGAAATTATCTTCTAAGCCAGATTATACTGAATCTAACCCAAATTACGATG
AACAAAAGTACATTCATGCTTTATCTCCATTGTTGGAACAAGAAGGTTGGCCAGGCG
CTAAGTTCATTGTTGATCAAGGTAGATCAGGTAAACAACCAACTGGTCAAAAAGCTT
GGGGTGATTGGTGTAATGCTCCAGGTACTGGTTTTGGTTTAAGACCATCTGCTAATA
CTGGTGATGCTTTGGTTGATGCTTTTGTTTGGGTTAAACCAGGTGGTGAATCTGATGG
TACTTCTGATACTTCTGCTGCAAGATATGATTATCATTGTGGTATTGATGGTGCTGTT
AAACCAGCTCCAGAAGCTGGTACTTGGTTTCAAGCTTACTTTGAACAATTGTTGAAG
AATGCTAATCCATCTTTCTTGTTATAAggcgcgccgaattcgagagactcgagactgaatcggatcgatcccggg
cccgtcgagggatctgcgatagatcaatttttttcttttctctttccccatcctttacgctaaaataatagtttattttattttttgaatattttttatt
tatatacgtatatatagactattatttatcttttaatgattattaagatttttattaaaaaaaaattcgctcctcttttaatgcctttatgcagtttttt
tttcccattcgatatttctatgttcgggttcagcgtattttaagtttaataactcgaaaattctgcgttcgttaaagcttgcatgcctgcaggtcgactc
tagaggatccccgggtaccgagctcgaattaattcgtaatcatggtcat
(SEQ ID NO: 38)
pRDH152
agctgtttcctgtgtgaaattgttatccgctcacaattccacacaacatacgagccggaagcataaagtgtaaagcctggggtgcctaatgagt
gaggtaactcacattaattgcgttgcgctcactgcccgctttccagtcgggaaacctgtcgtgccagctggattaatgaatcggccaacgcgc
ggggagaggcggtttgcgtattgggcgctcttccgcttcctcgctcactgactcgctgcgctcggtcgttcggctgcggcgagcggtatcag
ctcactcaaaggcggtaatacggttatccacagaatcaggggataacgcaggaaagaacatgtgagcaaaaggccagcaaaaggccagg
aaccgtaaaaaggccgcgttgctggcgtttttccataggctccgcccccctgacgagcatcacaaaaatcgacgctcaagtcagaggtggc
gaaacccgacaggactataaagataccaggcgtttccccctggaagctccctcgtgcgctctcctgttccgaccctgccgcttaccggatac
ctgtccgcctttctcccttcgggaagcgtggcgctttctcaatgctcacgctgtaggtatctcagttcggtgtaggtcgttcgctccaagctggg
ctgtgtgcacgaaccccccgttcagcccgaccgctgcgccttatccggtaactatcgtcttgagtccaacccggtaagacacgacttatcgcc
actggcagcagccactggtaacaggattagcagagcgaggtatgtaggcggtgctacagagttcttgaagtggtggcctaactacggctac
actagaaggacagtatttggtatctgcgctctgctgaagccagttaccttcggaaaaagagttggtagctcttgatccggcaaacaaaccacc
gctggtagcggtggtttttttgtttgcaagcagcagattacgcgcagaaaaaaaggatctcaagaagatcctttgatcttttctacggggtctga
cgctcagtggaacgaaaactcacgttaagggattttggtcatgagattatcaaaaaggatcttcacctagatccttttaaattaaaaatgaagtttt
aaatcaatctaaagtatatatgagtaaacttggtctgacagttaccaatgcttaatcagtgaggcacctatctcagcgatctgtctatttcgttcatc
catagttgcctgactccccgtcgtgtagataactacgatacgggagggcttaccatctggccccagtgctgcaatgataccgcgagacccac
gctcaccggctccagatttatcagcaataaaccagccagccggaagggccgagcgcagaagtggtcctgcaactttatccgcctccatcca
gtctattaattgttgccgggaagctagagtaagtagttcgccagttaatagtttgcgcaacgttgttgccattgctacaggcatcgtggtgtcac
gctcgtcgtttggtatggcttcattcagctccggttcccaacgatcaaggcgagttacatgatcccccatgttgtgcaaaaaagcggttagctc
cttcggtcctccgatcgttgtcagaagtaagttggccgcagtgttatcactcatggttatggcagcactgcataattctcttactgtcatgccatcc
gtaagatgcttttctgtgactggtgagtactcaaccaagtcattctgagaatagtgtatgcggcgaccgagttgctcttgcccggcgtcaatac
gggataataccgcgccacatagcagaactttaaaagtgctcatcattggaaaacgttcttcggggcgaaaactctcaaggatcttaccgctgtt
gagatccagttcgatgtaacccactcgtgcacccaactgatcttcagcatcttttactttcaccagcgtttctgggtgagcaaaaacaggaagg
caaaatgccgcaaaaaagggaataagggcgacacggaaatgttgaatactcatactcttcctttttcaatattattgaagcatttatcagggttat
tgtctcatgagcggatacatatttgaatgtatttagaaaaataaacaaataggggttccgcgcacatttccccgaaaagtgccacctgacgtcta
agaaaccattattatcatgacattaacctataaaaataggcgtatcacgaggccctttcgtctcgcgcgtttcggtgatgacggtgaaaacctct
gacacatgcagctcccggagacggtcacagcttgtctgtaagcggatgccgggagcagacaagcccgtcagggcgcgtcagcgggtgtt
ggcgggtgtcggggctggcttaactatgcggcatcagagcagattgtactgagagtgcaccataacgcatttaagcataaacacgcactatg
ccgttcttctcatgtatatatatatacaggcaacacgcagatataggtgcgacgtgaacagtgagctgtatgtgcgcagctcgcgttgcattttc
ggaagcgctcgttttcggaaacgctttgaagttcctattccgaagttcctattctctagctagaaagtataggaacttcagagcgcttttgaaaac
caaaagcgctctgaagacgcactttcaaaaaaccaaaaacgcaccggactgtaacgagctactaaaatattgcgaataccgcttccacaaa
cattgctcaaaagtatctctttgctatatatctctgtgctatatccctatataacctacccatccacctttcgctccttgaacttgcatctaaactcgac
ctctacattttttatgtttatctctagtattactctttagacaaaaaaattgtagtaagaactattcatagagtgaatcgaaaacaatacgaaaatgta
aacatttcctatacgtagtatatagagacaaaatagaagaaaccgttcataattttctgaccaatgaagaatcatcaacgctatcactttctgttca
caaagtatgcgcaatccacatcggtatagaatataatcggggatgcctttatcttgaaaaaatgcacccgcagcttcgctagtaatcagtaaac
gcgggaagtggagtcaggctttttttatggaagagaaaatagacaccaaagtagccttcttctaaccttaacggacctacagtgcaaaaagtt
atcaagagactgcattatagagcgcacaaaggagaaaaaaagtaatctaagatgctttgttagaaaaatagcgctctcgggatgcatttttgta
gaacaaaaaagaagtatagattctttgttggtaaaatagcgctctcgcgttgcatttctgttctgtaaaaatgcagctcagattctttgtttgaaaaa
ttagcgctctcgcgttgcatttttgttttacaaaaatgaagcacagattcttcgttggtaaaatagcgctttcgcgttgcatttctgttctgtaaaaat
gcagctcagattctttgtttgaaaaattagcgctctcgcgttgcatttttgttctacaaaatgaagcacagatgcttcgttaacaaagatatgctatt
gaagtgcaagatggaaacgcagaaaatgaaccggggatgcgacgtgcaagattacctatgcaatagatgcaatagtttctccaggaaccg
aaatacatacattgtcttccgtaaagcgctagactatatattattatacaggttcaaatatactatctgtttcagggaaaactcccaggttcggatgt
tcaaaattcaatgatgggtaacaagagcttttcaattcatcattttttttttattcttttttttgatttcggtttctttgaaatttttttgattcggtaa
tctccgaacagaaggaagaacgaaggaaggagcacagacttagattggtatatatacgcatatgtagtgttgaagaaacatgaaattgcccagtattct
taacccaactgcacagaacaaaaaccgaaacgaagataaatcatgtcgaaagctacatataaggaacgtgctgctactcatcctagtcctgtt
gctgccaagctatttaatatcatgcacgaaaagcaaacaaacttgtgtgcttcattggatgttcgtaccaccaaggaattactggagttagttga
agcattaggtcccaaaatttgtttactaaaaacacatgtggatatcttgactgatttttccatggagggcacagttaagccgctaaaggcattatc
cgccaagtacaattttttactcttcgaagacagaaaatttgctgacattggtaatacagtcaaattgcagtactctgcgggtgtatacagaatagc
agaatgggcagacattacgaatgcacacggtgtggtgggcccaggtattgttagcggtttgaagcaggcggcagaagaagtaacaaagga
acctagaggccttttgatgttagcagaattgtcatgcaagggctccctatctactggagaatatactaagggtactgttgacattgcgaagagc
gacaaagattttgttatcggctttattgctcaaagagacatgggtggaagagatgaaggttacgattggttgattatgacacccggtgtgggttt
agatgacaagggagacgcattgggtcaacagtatagaaccgtggatgatgtggtctctacaggatctgacattattattgttggaagaggact
atttgcaaagggaagggatgctaaggtagagggtgaacgttacagaaaagcaggctgggaagcatatttgagaagatgcggccagcaaa
actaaaaaactgtattataagtaaatgcatgtatactaaactcacaaattagagcttcaatttaattatatcagttattaccctatgcggtgtgaaat
accgcacagatgcgtaaggagaaaataccgcatcaggcgccattcgccattcaggctgcgcaactgttgggaagggcgatcggtgcggg
cctcttcgctattacgccagctggcgaaggggggatgtgctgcaaggcgattaagttgggtaacgccagggttttcccagtcacgacgttgt
aaaacgacggccagtgccaagctttctaactgatctatccaaaactgaaaattacattcttgattaggtttatcacaggcaaatgtaatttgtggt
attttgccgttcaaaatctgtagaattttctcattggtcacattacaacctgaaaatactttatctacaatcataccattcttataacatgtccccttaa
tactaggatcaggcatgaacgcatcacagacaaaatcttcttgacaaacgtcacaattgatccctccccatccgttatcacaatgacaggtgtca
ttttgtgctcttatgggacgatccttattaccgctttcatccggtgatagaccgccacagaggggcagagagcaatcatcacctgcaaaccctt
ctatacactcacatctaccagtgtacgaattgcattcagaaaactgtttgcattcaaaaataggtagcatacaattaaaacatggcgggcacgta
tcattgcccttatcttgtgcagttagacgcgaatttttcgaagaagtaccttcaaagaatggggtctcatcttgttttgcaagtaccactgagcag
gataataatagaaatgataatatactatagtagagataacgtcgatgacttcccatactgtaattgcttttagttgtgtatttttagtgtgcaagtttct
gtaaatcgattaatttttttttctttcctctttttattaaccttaatttttattttagattcctgacttcaactcaagacgcacagatattataacatct
gcacaataggcatttgcaagaattactcgtgagtaaggaaagagtgaggaactatcgcatacctgcatttaaagatgccgatttgggcgcgaatcctt
tattttggcttcaccctcatactattatcagggccagaaaaaggaagtgtttccctccttcttgaattgatgttaccctcataaagcacgtggcctc
ttatcgagaaagaaattaccgtcgctcgtgatttgtttgcaaaaagaacaaaactgaaaaaacccagacacgctcgacttcctgtcttcctattg
attgcagcttccaatttcgtcacacaacaaggtcctagcgacggctcacaggttttgtaacaagcaatcgaaggttctggaatggcgggaaa
gggtttagtaccacatgctatgatgcccactgtgatctccagagcaaagttcgttcgatcgtactgttactctctctctttcaaacagaattgtccg
aatcgtgtgacaacaacagcctgttctcacacactcttttcttctaaccaagggggtggtttagtttagtagaacctcgtgaaacttacatttacat
atatataaacttgcataaattggtcaatgcaagaaatacatatttggtcttttctaattcgtagtttttcaagttcttagatgctttctttttctctttt
ttacagatcatcaaggaagtaattatctactttttacaacaaatataaaacTTAATTAAAATGAAGTCTGCTGCTTTTTTG
GCTGCTTTAGCTGCTATTTTGCCAGCTTACGTTGCTGGTCAAGCTCAAACTTGGGCTC
AATGTGGTGGTATTGGTTTTACTGGTCCAACTACTTGTGTTGCTGGTTCTGTTTGTAC
TAAACAAAACGATTACTACTCTCAATGTATTCCAGGTTCTGCTACTACTCCAACTTCT
GCTCCAACATCTGCACCAACTTCTCAACCATCACAACCATCTTCTACTTCATCTGCTC
CATCTGGTCCATCTTCTACACCAACTCCATCTGCTAACAATCCATGGACTGGTTATCA
AATTTACTTGTCTCCATACTATGCTAATGAAGTTGCTGCAGCTGCTAAAGCTATTACT
GATCCAACTTTGGCTGCTAAAGCAGCTTCTGTTGCTAATATTCCAAATTTCACTTGGT
TGGATTCTGTTTCTAAAATTGCTGATTTGAAAACTTATTTGGCTGATGCTTCTGCTTT
GGGTAAATCTTCTGGTCAAAAGCAATTGTTGCAAATTGTTGTTTATGATTTGCCAGAT
AGAGATTGTGCTGCAAAAGCTTCTAATGGTGAATTTTCTATTGCTGATAATGGTTTGG
CTAACTACCAAAACTACATTGATCAAATTGTTGCTGCTGTTAAACAATTTCCAGATGT
TAGAGTTGTTGCTGTTATTGAACCAGATTCTTTGGCTAATTTGGTTACAAATTTAAAC
GTTCAAAAGTGTGCTAATGCTAAATCTACTTACTTGACTGCTGTTAATTATGCTTTGA
AGCAATTATCTTCTGTTGGTGTTTATCAATATATGGATGCTGGTCATGCTGGTTGGTT
GGGTTGGCCAGCTAATTTAACTCCAGCTGCTCAATTGTTTGCTCAAGTTTATTCTGAT
GCTGGTAAATCTCCATTCATTAAGGGTTTGGCTACTAATGTTGCTAATTACAATGCTT
TGTCTGCTGCTTCTCCAGATCCAATTACTCAAGGTGATCCAAATTACGATGAAATTCA
TTACATTAATGCTTTGGCTCCAGCTTTGCAATCTGCTGGTTTTCCAGCTACTTTTATTG
TTGATCAAGGTAGATCAGGTCAACAAAATCATAGACAACAATGGGGTGATTGGTGT
AACATTAAAGGTGCTGGTTTTGGTACTAGACCAACTACTAATACTGGTTCTTCTTTGA
TTGATTCTATTGTTTGGGTTAAACCAGGTGGTGAATCTGATGGTACTTCTAATTCTTC
ATCTCCAAGATTTGATTCTACTTGTTCTTTGTCTGATGCTACTCAACCAGCTCCAGAA
GCTGGTACTTGGTTTCAAGCTTACTTTGAAACTTTGGTTTCTAAAGCTAATCCACCAT
TGTTATAAGGCGCGCCgaattcgagagactcgagactgaatcggatcgatcccgggcccgtcgagggatctgcgatagatc
aatttttttcttttctctttccccatcctttacgctaaaataatagtttattttattttttgaatattttttatttatatacgtatatatagactattat
ttatcttttaatgattattaagatttttattaaaaaaaaattcgctcctcttttaatgcctttatgcagtttttttttcccattcgatatttctatgttc
gggttcagcgtattttaagtttaataactcgaaaattctgcgttcgttaaagcttgcatgcctgcaggtcgactctagaggatccccgggtaccgagctc
gaattaattcgtaatcatggtcat
(SEQ ID NO: 39)
pRDH153
agctgtttcctgtgtgaaattgttatccgctcacaattccacacaacatacgagccggaagcataaagtgtaaagcctggggtgcctaatgagt
gaggtaactcacattaattgcgttgcgctcactgcccgctttccagtcgggaaacctgtcgtgccagctggattaatgaatcggccaacgcgc
ggggagaggcggtttgcgtattgggcgctcttccgcttcctcgctcactgactcgctgcgctcggtcgttcggctgcggcgagcggtatcag
ctcactcaaaggcggtaatacggttatccacagaatcaggggataacgcaggaaagaacatgtgagcaaaaggccagcaaaaggccagg
aaccgtaaaaaggccgcgttgctggcgtttttccataggctccgcccccctgacgagcatcacaaaaatcgacgctcaagtcagaggtggc
gaaacccgacaggactataaagataccaggcgtttccccctggaagctccctcgtgcgctctcctgttccgaccctgccgcttaccggatac
ctgtccgcctttctcccttcgggaagcgtggcgctttctcaatgctcacgctgtaggtatctcagttcggtgtaggtcgttcgctccaagctggg
ctgtgtgcacgaaccccccgttcagcccgaccgctgcgccttatccggtaactatcgtcttgagtccaacccggtaagacacgacttatcgcc
actggcagcagccactggtaacaggattagcagagcgaggtatgtaggcggtgctacagagttcttgaagtggtggcctaactacggctac
actagaaggacagtatttggtatctgcgctctgctgaagccagttaccttcggaaaaagagttggtagctcttgatccggcaaacaaaccacc
gctggtagcggtggtttttttgtttgcaagcagcagattacgcgcagaaaaaaaggatctcaagaagatcctttgatcttttctacggggtctga
cgctcagtggaacgaaaactcacgttaagggattttggtcatgagattatcaaaaaggatcttcacctagatccttttaaattaaaaatgaagtttt
aaatcaatctaaagtatatatgagtaaacttggtctgacagttaccaatgcttaatcagtgaggcacctatctcagcgatctgtctatttcgttcatc
catagttgcctgactccccgtcgtgtagataactacgatacgggagggcttaccatctggccccagtgctgcaatgataccgcgagacccac
gctcaccggctccagatttatcagcaataaaccagccagccggaagggccgagcgcagaagtggtcctgcaactttatccgcctccatcca
gtctattaattgttgccgggaagctagagtaagtagttcgccagttaatagtttgcgcaacgttgttgccattgctacaggcatcgtggtgtcac
gctcgtcgtttggtatggcttcattcagctccggttcccaacgatcaaggcgagttacatgatcccccatgttgtgcaaaaaagcggttagctc
cttcggtcctccgatcgttgtcagaagtaagttggccgcagtgttatcactcatggttatggcagcactgcataattctcttactgtcatgccatcc
gtaagatgcttttctgtgactggtgagtactcaaccaagtcattctgagaatagtgtatgcggcgaccgagttgctcttgcccggcgtcaatac
gggataataccgcgccacatagcagaactttaaaagtgctcatcattggaaaacgttcttcggggcgaaaactctcaaggatcttaccgctgtt
gagatccagttcgatgtaacccactcgtgcacccaactgatcttcagcatcttttactttcaccagcgtttctgggtgagcaaaaacaggaagg
caaaatgccgcaaaaaagggaataagggcgacacggaaatgttgaatactcatactcttcctttttcaatattattgaagcatttatcagggttat
tgtctcatgagcggatacatatttgaatgtatttagaaaaataaacaaataggggttccgcgcacatttccccgaaaagtgccacctgacgtcta
agaaaccattattatcatgacattaacctataaaaataggcgtatcacgaggccctttcgtctcgcgcgtttcggtgatgacggtgaaaacctct
gacacatgcagctcccggagacggtcacagcttgtctgtaagcggatgccgggagcagacaagcccgtcagggcgcgtcagcgggtgtt
ggcgggtgtcggggctggcttaactatgcggcatcagagcagattgtactgagagtgcaccataacgcatttaagcataaacacgcactatg
ccgttcttctcatgtatatatatatacaggcaacacgcagatataggtgcgacgtgaacagtgagctgtatgtgcgcagctcgcgttgcattttc
ggaagcgctcgttttcggaaacgctttgaagttcctattccgaagttcctattctctagctagaaagtataggaacttcagagcgcttttgaaaac
caaaagcgctctgaagacgcactttcaaaaaaccaaaaacgcaccggactgtaacgagctactaaaatattgcgaataccgcttccacaaa
cattgctcaaaagtatctctttgctatatatctctgtgctatatccctatataacctacccatccacctttcgctccttgaacttgcatctaaactcgac
ctctacattttttatgtttatctctagtattactctttagacaaaaaaattgtagtaagaactattcatagagtgaatcgaaaacaatacgaaaatgta
aacatttcctatacgtagtatatagagacaaaatagaagaaaccgttcataattttctgaccaatgaagaatcatcaacgctatcactttctgttca
caaagtatgcgcaatccacatcggtatagaatataatcggggatgcctttatcttgaaaaaatgcacccgcagcttcgctagtaatcagtaaac
gcgggaagtggagtcaggctttttttatggaagagaaaatagacaccaaagtagccttcttctaaccttaacggacctacagtgcaaaaagtt
atcaagagactgcattatagagcgacaaaggagaaaaaaagtaatctaagatgctttgttagaaaaatagcgctctcgggatgcatttttgta
gaacaaaaaagaagtatagattctttgttggtaaaatagcgctctcgcgttgcatttctgttctgtaaaaatgcagctcagattctttgtttgaaaaa
ttagcgctctcgcgttgcatttttgttttacaaaaatgaagcacagattcttcgttggtaaaatagcgctttcgcgttgcatttctgttctgtaaaaat
gcagctcagattctttgtttgaaaaattagcgctctcgcgttgcatttttgttctacaaaatgaagcacagatgcttcgttaacaaagatatgctatt
gaagtgcaagatggaaacgcagaaaatgaaccggggatgcgacgtgcaagattacctatgcaatagatgcaatagtttctccaggaaccg
aaatacatacattgtcttccgtaaagcgctagactatatattattatacaggttcaaatatactatctgtttcagggaaaactcccaggttcggatgt
tcaaaattcaatgatgggtaacaagagcttttcaattcatcattttttttttattcttttttttgatttcggtttctttgaaatttttttgattcggtaa
tctccgaacagaaggaagaacgaaggaaggagcacagacttagattggtatatatacgcatatgtagtgttgaagaaacatgaaattgcccagtattct
taacccaactgcacagaacaaaaaccgaaacgaagataaatcatgtcgaaagctacatataaggaacgtgctgctactcatcctagtcctgtt
gctgccaagctatttaatatcatgcacgaaaagcaaacaaacttgtgtgcttcattggatgttcgtaccaccaaggaattactggagttagttga
agcattaggtcccaaaatttgtttactaaaaacacatgtggatatcttgactgatttttccatggagggcacagttaagccgctaaaggcattatc
cgccaagtacaattttttactcttcgaagacagaaaatttgctgacattggtaatacagtcaaattgcagtactctgcgggtgtatacagaatagc
agaatgggcagacattacgaatgcacacggtgtggtgggcccaggtattgttagcggtttgaagcaggcggcagaagaagtaacaaagga
acctagaggccttttgatgttagcagaattgtcatgcaagggctccctatctactggagaatatactaagggtactgttgacattgcgaagagc
gacaaagattttgttatcggctttattgctcaaagagacatgggtggaagagatgaaggttacgattggttgattatgacacccggtgtgggttt
agatgacaagggagacgcattgggtcaacagtatagaaccgtggatgatgtggtctctacaggatctgacattattattgttggaagaggact
atttgcaaagggaagggatgctaaggtagagggtgaacgttacagaaaagcaggctgggaagcatatttgagaagatgcggccagcaaa
actaaaaaactgtattataagtaaatgcatgtatactaaactcacaaattagagcttcaatttaattatatcagttattaccctatgcggtgtgaaat
accgcacagatgcgtaaggagaaaataccgcatcaggcgccattcgccattcaggctgcgcaactgttgggaagggcgatcggtgcggg
cctcttcgctattacgccagctggcgaaggggggatgtgctgcaaggcgattaagttgggtaacgccagggttttcccagtcacgacgttgt
aaaacgacggccagtgccaagctttctaactgatctatccaaaactgaaaattacattcttgattaggtttatcacaggcaaatgtaatttgtggt
attttgccgttcaaaatctgtagaattttctcattggtcacattacaacctgaaaatactttatctacaatcataccattcttataacatgtccccttaa
tactaggatcaggcatgaacgcatcacagacaaaatcttcttgacaaacgtcacaattgatccctccccatccgttatcacaatgacaggtgtca
ttttgtgctcttatgggacgatccttattaccgctttcatccggtgatagaccgccacagaggggcagagagcaatcatcacctgcaaaccctt
ctatacactcacatctaccagtgtacgaattgcattcagaaaactgtttgcattcaaaaataggtagcatacaattaaaacatggcgggcacgta
tcattgcccttatcttgtgcagttagacgcgaatttttcgaagaagtaccttcaaagaatggggtctcatcttgttttgcaagtaccactgagcag
gataataatagaaatgataatatactatagtagagataacgtcgatgacttcccatactgtaattgcttttagttgtgtatttttagtgtgcaagtttct
gtaaatcgattaatttttttttctttcctctttttattaaccttaatttttattttagattcctgacttcaactcaagacgcacagatattataacatct
gcacaataggcatttgcaagaattactcgtgagtaaggaaagagtgaggaactatcgcatacctgcatttaaagatgccgatttgggcgcgaatcctt
tattttggcttcaccctcatactattatcagggccagaaaaaggaagtgtttccctccttcttgaattgatgttaccctcataaagcacgtggcctc
ttatcgagaaagaaattaccgtcgctcgtgatttgtttgcaaaaagaacaaaactgaaaaaacccagacacgctcgacttcctgtcttcctattg
attgcagcttccaatttcgtcacacaacaaggtcctagcgacggctcacaggttttgtaacaagcaatcgaaggttctggaatggcgggaaa
gggtttagtaccacatgctatgatgcccactgtgatctccagagcaaagttcgttcgatcgtactgttactctctctctttcaaacagaattgtccg
aatcgtgtgacaacaacagcctgttctcacacactcttttcttctaaccaagggggtggtttagtttagtagaacctcgtgaaacttacatttacat
atatataaacttgcataaattggtcaatgcaagaaatacatatttggtcttttctaattcgtagtttttcaagttcttagatgctttctttttctctttt
ttacagatcatcaaggaagtaattatctactttttacaacaaatataaaacttaattaaAATGTCTAGATTCTCTGCTTTGACTG
CTTTGTTGTTGTCTTTGCCATTGTTGGCTATTGCTCAATCTCCATTGTATGGTCAATGT
GGTGGTAATGGTTGGACTGGTCCAAAAACTTGTGTTTCTGGTGCTACTTGTACTGTTA
TTAATGATTGGTATTGGCAATGTTTGCCAGGTAATGGTCCAACTTCTTCTTCTCCAAC
TTCTACTCCAACTACAACTACTACTACTGGTGGTCCACAACCAACTGTTCCAGCTGCT
GGTAATCCATATACTGGTTACGAAATTTACTTGTCTCCATATTATGCTGCTGAAGCTC
AAGCTGCTGCTGCTCAAATTTCTGATGCTACTCAAAAAGCTAAAGCTTTGAAAGTTG
CTCAAATTCCAACTTTTACTTGGTTTGATGTTATTGCTAAAACTTCTACTTTGGGTGA
TTATTTGGCTGAAGCTTCTGCTTTGGGTAAATCTTCTGGTAAAAAGTACTTGGTTCAA
ATTGTTGTTTATGATTTGCCAGATAGAGATTGTGCTGCTTTGGCTTCTAATGGTGAAT
TTTCTATTGCTAACAACGGTTTGAACAATTACAAAGGTTACATTGATCAATTGGTTGC
ACAAATTAAGAAATACCCAGATGTTAGAGTTGTTGCTGTTATTGAACCAGATTCTTT
GGCTAATTTGGTTACAAATTTGAACGTTTCTAAGTGTGCTAATGCTCAAACTGCTTAC
AAAGCTGGTGTTACTTATGCTTTGCAACAATTGAACTCTGTTGGTGTTTACATGTATT
TGGATGCTGGTCATGCTGGTTGGTTGGGTTGGCCAGCTAATTTGAATCCAGCTGCTC
AATTGTTTTCTCAATTGTATAGAGATGCTGGTTCTCCACAATACGTTAGAGGTTTGGC
TACTAATGTTGCTAATTACAATGCTTTGTCTGCTTCTTCACCAGATCCAGTTACTCAA
GGTAATCCAAATTACGATGAATTGCATTACATTAATGCTTTGGCTCCAGCTTTGCAAT
CTGGTGGTTTTCCAGCTCATTTTATTGTTGATCAAGGTAGATCAGGTGTTCAAAACAT
TAGACAACAATGGGGTGATTGGTGTAATGTTAAAGGTGCTGGTTTTGGTCAAAGACC
AACTTTATCTACTGGTTCTTCTTTGATTGATGCTATTGTTTGGATTAAACCAGGTGGT
GAATGTGATGGTACTACTAATACATCTTCTCCAAGATATGATTCTCATTGTGGTTTGT
CTGATGCTACTCCAAATGCTCCTGAAGCTGGTCAATGGTTTCAAGCTTACTTTGAAAC
TTTGGTTAGAAATGCTTCTCCACCATTGTTATAAggcgcgccgaattcgagagactcgagactgaatcgga
tcgatcccgggcccgtcgagggatctgcgatagatcaatttttttcttttctctttccccatcctttacgctaaaataatagtttattttattttttgaa
tattttttatttatatacgtatatatagactattatttatcttttaatgattattaagatttttattaaaaaaaaattcgctcctcttttaatgccttta
tgcagtttttttttcccattcgatatttctatgttcgggttcagcgtattttaagtttaataactcgaaaattctgcgttcgttaaagcttgcatgcctg
caggtcgactctagaggatccccgggtaccgagctcgaattaattcgtaatcatggtcat
(SEQ ID NO: 40)
pRDH154
agctgtttcctgtgtgaaattgttatccgctcacaattccacacaacatacgagccggaagcataaagtgtaaagcctggggtgcctaatgagt
gaggtaactcacattaattgcgttgcgctcactgcccgctttccagtcgggaaacctgtcgtgccagctggattaatgaatcggccaacgcgc
ggggagaggcggtttgcgtattgggcgctcttccgcttcctcgctcactgactcgctgcgctcggtcgttcggctgcggcgagcggtatcag
ctcactcaaaggcggtaatacggttatccacagaatcaggggataacgcaggaaagaacatgtgagcaaaaggccagcaaaaggccagg
aaccgtaaaaaggccgcgttgaggcgtttttccataggctccgcccccctgacgagcatcacaaaaatcgacgctcaagtcagaggtggc
gaaacccgacaggactataaagataccaggcgtttccccctggaagctccctcgtgcgctctcctgttccgaccctgccgcttaccggatac
ctgtccgcctttctcccttcgggaagcgtggcgctttctcaatgctcacgctgtaggtatctcagttcggtgtaggtcgttcgctccaagctggg
ctgtgtgcacgaaccccccgttcagcccgaccgctgcgccttatccggtaactatcgtcttgagtccaacccggtaagacacgacttatcgcc
actggcagcagccactggtaacaggattagcagagcgaggtatgtaggcggtgctacagagttcttgaagtggtggcctaactacggctac
actagaaggacagtatttggtatctgcgctctgctgaagccagttaccttcggaaaaagagttggtagctcttgatccggcaaacaaaccacc
gctggtagcggtggtttttttgtttgcaagcagcagattacgcgcagaaaaaaaggatctcaagaagatcctttgatcttttctacggggtctga
cgctcagtggaacgaaaactcacgttaagggattttggtcatgagattatcaaaaaggatcttcacctagatccttttaaattaaaaatgaagtttt
aaatcaatctaaagtatatatgagtaaacttggtctgacagttaccaatgcttaatcagtgaggcacctatctcagcgatctgtctatttcgttcatc
catagttgcctgactccccgtcgtgtagataactacgatacgggagggcttaccatctggccccagtgctgcaatgataccgcgagacccac
gctcaccggctccagatttatcagcaataaaccagccagccggaagggccgagcgcagaagtggtcctgcaactttatccgcctccatcca
gtctattaattgttgccgggaagctagagtaagtagttcgccagttaatagtttgcgcaacgttgttgccattgctacaggcatcgtggtgtcac
gctcgtcgtttggtatggcttcattcagctccggttcccaacgatcaaggcgagttacatgatcccccatgttgtgcaaaaaagcggttagctc
cttcggtcctccgatcgttgtcagaagtaagttggccgcagtgttatcactcatggttatggcagcactgcataattctcttactgtcatgccatcc
gtaagatgcttttctgtgactggtgagtactcaaccaagtcattctgagaatagtgtatgcggcgaccgagttgctcttgcccggcgtcaatac
gggataataccgcgccacatagcagaactttaaaagtgctcatcattggaaaacgttcttcggggcgaaaactctcaaggatcttaccgctgtt
gagatccagttcgatgtaacccactcgtgcacccaactgatcttcagcatcttttactttcaccagcgtttctgggtgagcaaaaacaggaagg
caaaatgccgcaaaaaagggaataagggcgacacggaaatgttgaatactcatactcttcctttttcaatattattgaagcatttatcagggttat
tgtctcatgagcggatacatatttgaatgtatttagaaaaataaacaaataggggttccgcgcacatttccccgaaaagtgccacctgacgtcta
agaaaccattattatcatgacattaacctataaaaataggcgtatcacgaggccctttcgtctcgcgcgtttcggtgatgacggtgaaaacctct
gacacatgcagctcccggagacggtcacagcttgtctgtaagcggatgccgggagcagacaagcccgtcagggcgcgtcagcgggtgtt
ggcgggtgtcggggctggcttaactatgcggcatcagagcagattgtactgagagtgcaccataacgcatttaagcataaacacgcactatg
ccgttcttctcatgtatatatatatacaggcaacacgcagatataggtgcgacgtgaacagtgagctgtatgtgcgcagctcgcgttgcattttc
ggaagcgctcgttttcggaaacgctttgaagttcctattccgaagttcctattctctagctagaaagtataggaacttcagagcgcttttgaaaac
caaaagcgctctgaagacgcactttcaaaaaaccaaaaacgcaccggactgtaacgagctactaaaatattgcgaataccgcttccacaaa
cattgctcaaaagtatctctttgctatatatctctgtgctatatccctatataacctacccatccacctttcgctccttgaacttgcatctaaactcgac
ctctacattttttatgtttatctctagtattactctttagacaaaaaaattgtagtaagaactattcatagagtgaatcgaaaacaatacgaaaatgta
aacatttcctatacgtagtatatagagacaaaatagaagaaaccgttcataattttctgaccaatgaagaatcatcaacgctatcactttctgttca
caaagtatgcgcaatccacatcggtatagaatataatcggggatgcctttatcttgaaaaaatgcacccgcagcttcgctagtaatcagtaaac
gcgggaagtggagtcaggctttttttatggaagagaaaatagacaccaaagtagccttcttctaaccttaacggacctacagtgcaaaaagtt
atcaagagactgcattatagagcgcacaaaggagaaaaaaagtaatctaagatgctttgttagaaaaatagcgctctcgggatgcatttttgta
gaacaaaaaagaagtatagattctttgttggtaaaatagcgctctcgcgttgcatttctgttctgtaaaaatgcagctcagattctttgtttgaaaaa
ttagcgctctcgcgttgcatttttgttttacaaaaatgaagcacagattcttcgttggtaaaatagcgctttcgcgttgcatttctgttctgtaaaaat
gcagctcagattctttgtttgaaaaattagcgctctcgcgttgcatttttgttctacaaaatgaagcacagatgcttcgttaacaaagatatgctatt
gaagtgcaagatggaaacgcagaaaatgaaccggggatgcgacgtgcaagattacctatgcaatagatgcaatagtttctccaggaaccg
aaatacatacattgtcttccgtaaagcgctagactatatattattatacaggttcaaatatactatctgtttcagggaaaactcccaggttcggatgt
tcaaaattcaatgatgggtaacaagagcttttcaattcatcattttttttttattcttttttttgatttcggtttctttgaaatttttttgattcggtaa
tctccgaacagaaggaagaacgaaggaaggagcacagacttagattggtatatatacgcatatgtagtgttgaagaaacatgaaattgcccagtattct
taacccaactgcacagaacaaaaaccgaaacgaagataaatcatgtcgaaagctacatataaggaacgtgctgctactcatcctagtcctgtt
gctgccaagctatttaatatcatgcacgaaaagcaaacaaacttgtgtgcttcattggatgttcgtaccaccaaggaattactggagttagttga
agcattaggtcccaaaatttgtttactaaaaacacatgtggatatcttgactgatttttccatggagggcacagttaagccgctaaaggcattatc
cgccaagtacaattttttactcttcgaagacagaaaatttgctgacattggtaatacagtcaaattgcagtactctgcgggtgtatacagaatagc
agaatgggcagacattacgaatgcacacggtgtggtgggcccaggtattgttagcggtttgaagcaggcggcagaagaagtaacaaagga
acctagaggccttttgatgttagcagaattgtcatgcaagggctccctatctactggagaatatactaagggtactgttgacattgcgaagagc
gacaaagattttgttatcggctttattgctcaaagagacatgggtggaagagatgaaggttacgattggttgattatgacacccggtgtgggttt
agatgacaagggagacgcattgggtcaacagtatagaaccgtggatgatgtggtctctacaggatctgacattattattgttggaagaggact
atttgcaaagggaagggatgctaaggtagagggtgaacgttacagaaaagcaggctgggaagcatatttgagaagatgcggccagcaaa
actaaaaaactgtattataagtaaatgcatgtatactaaactcacaaattagagcttcaatttaattatatcagttattaccctatgcggtgtgaaat
accgcacagatgcgtaaggagaaaataccgcatcaggcgccattcgccattcaggctgcgcaactgttgggaagggcgatcggtgcggg
cctcttcgctattacgccagctggcgaaggggggatgtgctgcaaggcgattaagttgggtaacgccagggttttcccagtcacgacgttgt
aaaacgacggccagtgccaagctttctaactgatctatccaaaactgaaaattacattcttgattaggtttatcacaggcaaatgtaatttgtggt
attttgccgttcaaaatctgtagaattttctcattggtcacattacaacctgaaaatactttatctacaatcataccattcttataacatgtccccttaa
tactaggatcaggcatgaacgcatcacagacaaaatcttcttgacaaacgtcacaattgatccctccccatccgttatcacaatgacaggtgtca
ttttgtgctcttatgggacgatccttattaccgctttcatccggtgatagaccgccacagaggggcagagagcaatcatcacctgcaaaccctt
ctatacactcacatctaccagtgtacgaattgcattcagaaaactgtttgcattcaaaaataggtagcatacaattaaaacatggcgggcacgta
tcattgcccttatcttgtgcagttagacgcgaatttttcgaagaagtaccttcaaagaatggggtctcatcttgttttgcaagtaccactgagcag
gataataatagaaatgataatatactatagtagagataacgtcgatgacttcccatactgtaattgcttttagttgtgtatttttagtgtgcaagtttct
gtaaatcgattaatttttttttctttcctctttttattaaccttaatttttattttagattcctgacttcaactcaagacgcacagatattataacatct
gcacaataggcatttgcaagaattactcgtgagtaaggaaagagtgaggaactatcgcatacctgcatttaaagatgccgatttgggcgcgaatcctt
tattttggcttcaccctcatactattatcagggccagaaaaaggaagtgtttccctccttcttgaattgatgttaccctcataaagcacgtggcctc
ttatcgagaaagaaattaccgtcgctcgtgatttgtttgcaaaaagaacaaaactgaaaaaacccagacacgctcgacttcctgtcttcctattg
attgcagcttccaatttcgtcacacaacaaggtcctagcgacggctcacaggttttgtaacaagcaatcgaaggttctggaatggcgggaaa
gggtttagtaccacatgctatgatgcccactgtgatctccagagcaaagttcgttcgatcgtactgttactctctctctttcaaacagaattgtccg
aatcgtgtgacaacaacagcctgttctcacacactcttttcttctaaccaagggggtggtttagtttagtagaacctcgtgaaacttacatttacat
atatataaacttgcataaattggtcaatgcaagaaatacatatttggtcttttctaattcgtagtttttcaagttcttagatgctttctttttctctttt
ttacagatcatcaaggaagtaattatctactttttacaacaaatataaaacttaattaaAATGAAGGCTTCTATTGCTTTGACTG
CTATTGCTGCTTTGGCTGCTAATGCTTCTGCTGCTTGTTTTTCTGAAAGATTGGGTTAT
CCATGTTGTAGAGGTAATGAAGTTTTCTACACTGATAATGATGGTGATTGGGGTGTT
GAAAATGGTAATTGGTGTGGTATTGGTGGTGCTTCTGCTACTACTTGTTGGTCACAA
GCTTTAGGTTACCCTTGTTGTACTTCTACTTCTGATGTTGCTTACGTTGATGGTGACG
GTAACTGGGGTGTCGAAAACGGTAACTGGTGCGGTATAATTGCAGGTGGTAATTCTT
CTAACAACAACTCTGGTTCTACTATTAATGTTGGTGATGTTACTATTGGTAACCAATA
CACTCATACTGGTAATCCATTTGCTGGTCATAAATTCTTTATTAACCCATACTATACT
GCTGAAGTTGATGGTGCTATTGCTCAAATTTCTAATGCTTCTTTGAGAGCTAAAGCTG
AAAAGATGAAAGAATTTTCTAACGCTATTTGGTTGGATACTATTAAGAATATGAACG
AATGGTTGGAAAAGAATTTGAAATATGCTTTGGCTGAACAAAATGAAACTGGTAAG
ACTGTTTTGACAGTTTTTGTTGTTTATGATTTGCCAGGTAGAGATTGTCATGCTTTAG
CTTCTAATGGTGAATTGTTGGCTAATGATTCTGATTGGGCAAGATATCAATCTGAAT
ACATTGATGTTATTGAAGAAAAGTTGAAAACTTACAAGTCTCAACCAGTTGTTTTGG
TTGTTGAACCAGATTCTTTGGCTAATATGGTTACAAATTTGGATTCTACTCCAGCTTG
TAGAGATTCTGAAAAATACTATATGGATGGTCATGCTTACTTGATTAAAAAGTTGGG
TGTTTTGCCACATGTTGCAATGTATTTGGATATTGGTCATGCTTTTTGGTTGGGTTGG
GATGATAATAGATTGAAAGCTGGTAAAGTTTACTCTAAGGTTATTCAATCTGGTGCT
CCAGGTAATGTTAGAGGTTTTGCTTCTAATGTTGCTAATTATACTCCATGGGAAGATC
CAACTTTGTCTAGAGGTCCAGATACTGAATGGAATCCATGTCCAGATGAAAAAAGAT
ACATTGAAGCAATGTACAAAGATTTTAAGTCTGCTGGTATTAAGTCTGTTTACTTCAT
TGATGATACTTCTAGAAATGGTCATAAGACTGATAGAACTCATCCAGGTGAATGGTG
TAATCAAACAGGTGTTGGTATTGGTGCTAGACCACAAGCTAATCCAATTTCTGGTAT
GGATTACTTGGATGCTTTTTATTGGGTTAAACCATTGGGTGAATCTGATGGTTATTCT
GATACTACTGCTGTCAGATATGATGGTTATTGTGGTCATGCTACTGCTATGAAACCA
GCTCCTGAAGCTGGTCAATGGTTTCAAAAACATTTCGAACAAGGTTTGGAAAATGCT
AATCCACCATTGTTATAAggcgcgccgaattcgagagactcgagactgaatcggatcgatcccgggcccgtcgagggat
ctgcgatagatcaatttttttcttttctctttccccatcctttacgctaaaataatagtttattttattttttgaatattttttatttatatacgtatat
atagactattatttatcttttaatgattattaagatttttattaaaaaaaaattcgctcctcttttaatgcctttatgcagtttttttttcccattcgat
atttctatgttcgggttcagcgtattttaagtttaataactcgaaaattctgcgttcgttaaagcttgcatgcctgcaggtcgactctagaggatccccg
ggtaccgagctcgaattaattcgtaatcatggtcat
(SEQ ID NO: 41)

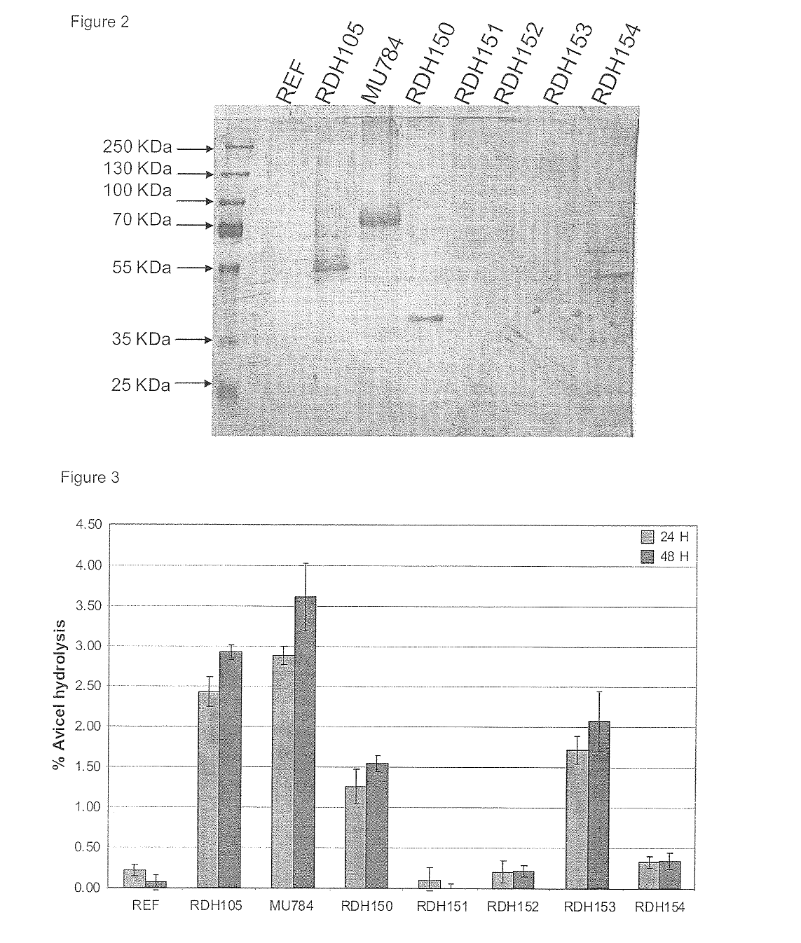

The plasmids were all transformed to S. cerevisiae (strain Y294), and transformants were confirmed with PCR. Along with the reference strain containing a plasmid without a heterologous cellulase and a strain expressing the Clcbh2b (pMU784), the five cbh2 containing strains were tested for protein production. The strains were grown in double strength SC^{-URA} medium (3.4 g/L YNB; 3 g/L amino acid dropout pool without uracil; 10 g/L ammonium sulfate; 20 g/L glucose) that was buffered to pH 6 (20 g/L succinic acid; 12 g/L NaOH, set pH to 6 with NaOH). Glucose was added after autoclaving of the other components from a 50% glucose stock solution. 10 mL cultures in 125 mL Erlenmeyer flasks were grown at 30° C. for three days. Three flasks were inoculated for each strain. After incubation, samples were taken for analysis. After centrifugation of the samples, 12 μl of each was taken, added to 5 μl of protein loading buffer and boiled for 5 minutes. The samples were subsequently loaded on a 10% SDS-PAGE and separated, followed by silver staining (FIG. 2).

The theoretical enzyme size was estimated for each of the heterologous genes using the Compute pI/Mw tool available at http://ca.expasy.org/tools/pi_tool.html. The results are listed in Table 5. FIG. 2 shows that bands in the expected size range were visible for C. heterostrophus CEL7 (pRDH151) and Piromyces sp. CEL6A (pRDH154). In addition, V. volvacea CBHII-I appears as a diffuse band in the 130 KDa range. This size is greater than the predicted enzyme size, and the diffuse band was seen on several gels.

### Example 2

### Avicel Hydrolysis in Yeast Expressing a Heterologous Cbh2

All strains were then tested for activity using the high-throughput Avicel conversion method using an Avicel concentration of 1% (or 10 g/L). The Dintrosalicylic Acid Reagent Solution (DNS) used for the assay procedure contained phenol which, according to literature, renders greater sensitivity. Activity data can be seen in FIG. 3. From the activity data it is apparent that the strain expressing C. heterostrophus CEL7 (pRDH150) and V. volvacea CBHII-I (pRDH153) yielded appreciable amounts of activity on Avicel. The Piromyces sp. CEL6A-expressing strain also showed some activity.

### Example 3

### Specific Activity of Cbh2s Expressed Heterologously in Yeast

To estimate the specific activity of the Cbh2s, the Bradford method (BioRad protein assay) was used as it is prescribed for microtiter plates, using the Gamma globulin standard. Supernatants samples were first subjected to the buffer exchange procedure as directed for the 2 mL Zeba desalt spin columns (Thermo Scientific). The amount of protein detected by the protein assay seemed to agree with what was seen on the SDS-PAGE.

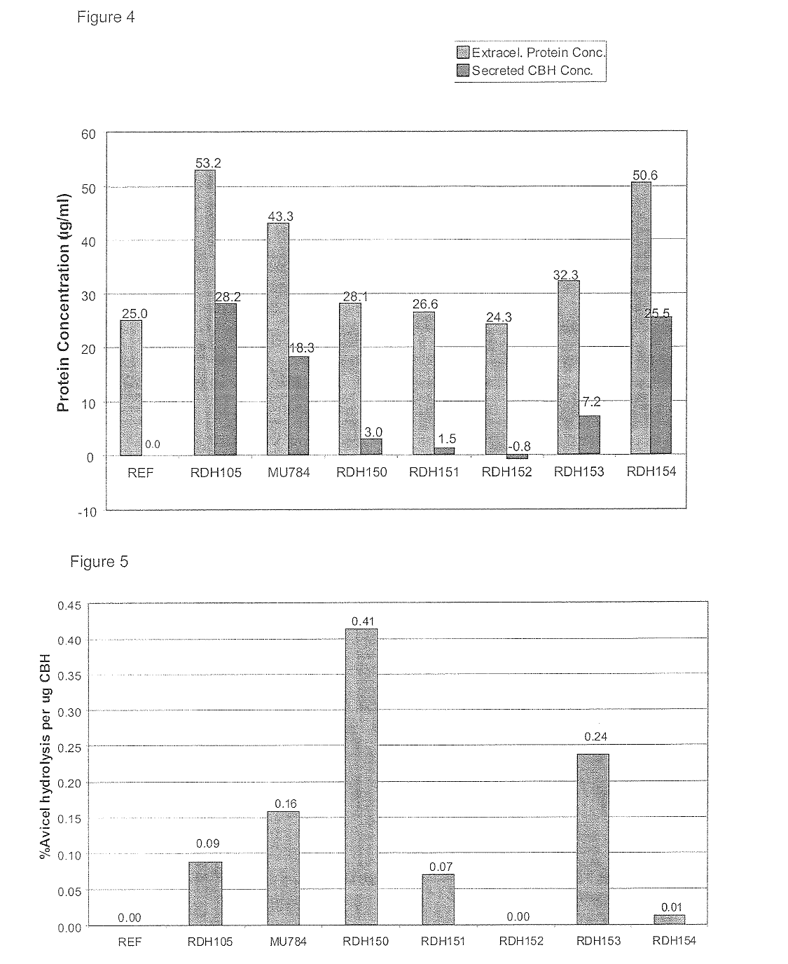

The average amount of protein present in the REF strain samples was then subtracted from the amount of protein measured in the other samples to give an indication of the amount of heterologously expressed Cbh2 that was present in each sample (FIG. 4). Next, the specific activity of each CBH was estimated by dividing the activity (FIG. 3) by the amount of CBH present (FIG. 4) and expressed in “percentage degradation per μg protein” (FIG. 5). C. heterostrophus CEL7 (pRDH150) and V. volvacea CBHII-I (pRDH153) had 2.6 times and 1.5 times greater specific activity than ClCbh2b on Avicel.

These examples illustrate possible embodiments of the present invention. While the invention has been particularly shown and described with reference to some embodiments thereof, it will be understood by those skilled in the art that they have been presented by way of example only, and not limitation, and various changes in form and details can be made therein without departing from the spirit and scope of the invention. Thus, the breadth and scope of the present invention should not be limited by any of the above-described exemplary embodiments, but should be defined only in accordance with the following claims and their equivalents.

All documents cited herein, including journal articles or abstracts, published or corresponding U.S. or foreign patent applications, issued or foreign patents, or any other documents, are each entirely incorporated by reference herein, including all data, tables, figures, and text presented in the cited documents.

1. An isolated polynucleotide comprising a nucleic acid which encodes a cellobiohydrolase or a fragment thereof, wherein said nucleic acid is codon-optimized for expression in a yeast strain and wherein the cellobiohydrolase is selected from the group consisting of a: Cochliobolus heterostrophus; Gibberella zeae; Irpex lacteus; Volvariella; and Piromyces sp. cellobiohydrolase.

2. (canceled)

3. (canceled)

4. (canceled)

5. (canceled)

6. (canceled)

7. (canceled)

8. (canceled)

9. (canceled)

10. (canceled)

11. The polynucleotide of claim 1, wherein the fragment of the cellobiohydrolase is a cellobiohydrolase signal peptide.

12. (canceled)

13. The polynucleotide of claim 1, wherein the fragment of the cellobiohydrolase is a cellobiohydrolase CBM.

14. (canceled)

15. The polynucleotide of claim 1, wherein the fragment of the cellobiohydrolase is a GH family 6 domain.

16. (canceled)

17. (canceled)

18. (canceled)

19. (canceled)

20. (canceled)

21. (canceled)

22. (canceled)

23. (canceled)

24. (canceled)

25. (canceled)

26. The polynucleotide of claim 1, wherein said polynucleotide is operably associated with a heterologous nucleic acid.

27. The polynucleotide of claim 26, wherein the heterologous nucleic acid encodes a signal peptide, a secretion signal, or a carbohydrate binding module.

28. (canceled)

29. (canceled)

30. (canceled)

31. (canceled)

32. (canceled)

33. (canceled)

34. A vector comprising the polynucleotide of claim 1.

35. The vector of claim 34 further comprising an S. cerevisiae PGK1 promoter, an S. cerevisiae PGK1 terminator or both an S. cerevisiae PGK1 promoter, and an S. cerevisiae PGK1 terminator.

36. (canceled)

37. (canceled)

38. A host cell comprising a polynucleotide comprising a nucleic acid which encodes a heterologous cellobiohydrolase or fragment thereof, wherein said heterologous cellobiohydrolase is selected from the group consisting of: a Cochliobolus heterostrophus; Gibberella zeae; Irpex lacteus; Volvariella volvacea; and Piromyces sp. cellobiohydrolase.

39. The host cell of claim 38, wherein the cellobiohydrolase is a cellobiohydrolase II.

40. The host cell of claim 39, wherein the cellobiohydrolase II is selected from the group consisting of Cochliobolus heterostrophus C4 cel7; Gibberella zeae K59 cel6; Irpex lacteus MC-2 cex3; Volvariella volvacea cbhII-I; and Piromyces sp. E2 cel6A.

41. (canceled)

42. (canceled)

43. (canceled)

44. (canceled)

45. A host cell comprising the polynucleotide of claim 1.

46. (canceled)

47. (canceled)

48. (canceled)

49. (canceled)

50. (canceled)

51. (canceled)

52. The host cell of claim 38 further comprising one or more additional heterologous cellulases selected from the group consisting of an endogluconase, a β-glucosidase, and a cellobiohydrolase.

53. (canceled)

54. The host cell of claim 52, wherein the host cell comprises: (i) a heterologous polynucleotide encoding C. formosanus endoglucanase I; (ii) a heterologous polynucleotide encoding S. fibuligera β-glucosidase I; (iii) a heterologous polynucleotide encoding T. emersonii cellobiohydrolase I; and (iv) a heterologous polynucleotide encoding Cochliobolus heterostrophus C4 cel7; Gibberella zeae K59 cel6; Irpex lacteus MC-2 cex3; Volvariella volvacea cbhII-I; or Piromyces sp. E2 cel6A.

55. The host cell of claim 38, wherein the host cell can saccharify crystalline cellulose.

56. (canceled)

57. A host cell comprising a heterologous polynucleotide encoding a cellobiohydrolase, wherein the cellobiohydrolase has a specific activity on Avicel of at least about 0.08 μmol/mg/min, and wherein the host cell is a yeast cell.

58. (canceled)

59. (canceled)

60. (canceled)

61. (canceled)

62. (canceled)

63. (canceled)

64. A co-culture comprising the host cell of claim 38 and a second host cell comprising a heterologous cellulase selected from the group consisting of an endogluconase, a β-glucosidase, and a cellobiohydrolase.

65. (canceled)

66. A method for hydrolyzing a cellulosic substrate, comprising contacting said cellulosic substrate with the host cell of claim 38.

67. (canceled)

68. A method of fermenting cellulose comprising culturing the host cell of claim 38 in medium that contains crystalline cellulose under suitable conditions for a period sufficient to allow saccharification and fermentation of the cellulose.

69. The method of claim 68, wherein said host cell produces ethanol.
